(19) United States
(12) Patent Application Publication
(10) Pub. No.: US 2023/0285576 A1
(43) Pub. Date: Sep. 14, 2023

(54) TREATMENT OF CANCER USING A CYCLODEXTRIN-CONTAINING POLYMER-TOPOISOMERASE INHIBITOR CONJUGATE AND A PARP INHIBITOR

(71) Applicant: ELLIPSES PHARMA LTD, London (GB)

(72) Inventors: Rajan JETHWA, London (GB); Rochana WICKRAMASINGHE, London (GB); Geoff FISHER, London (GB); Sital PATEL, London (GB)

(21) Appl. No.: 18/019,792

(22) PCT Filed: Aug. 5, 2021
(86) PCT No.: PCT/EP2021/071856

§ 371 Date: Feb. 3, 2023

(30) Foreign Application Priority Data
Aug. 5, 2020 (EP) 20189707.1

(51) Int. Cl. A61K 47/61; A61K 31/502; A61K 31/4184; A61K 31/454; A61K 31/55; A61K 31/5025; A61P 35/00; A61P 15/00

(52) U.S. Cl. CPC A61K 47/61; A61K 31/502; A61K 31/4184; A61K 31/454; A61K 31/55; A61K 31/5025; A61P 35/00; A61P 15/00

(57) ABSTRACT

A cyclodextrin-containing polymer-topoisomerase inhibitor conjugate for use in treating ovarian cancer in a subject. The use is in combination with a poly (ADP-ribose) polymerase (PARP) inhibitor. The subject has previously undergone chemotherapy comprising a platinum-based chemotherapeutic agent prior to said use. In one embodiment, CRLX-101, which is a conjugate of a cyclodextrin-containing polymer to camptothecin, is used in combination with olaparib.

### TECHNICAL FIELD

The present application relates to the treatment of cancer. The application relates more particularly, but not necessarily exclusively, to the treatment of advanced ovarian cancer in patients who have previously undergone a therapy comprising a platinum-based chemotherapeutic agent.

### BACKGROUND

There are approximately 300,000 new cases of ovarian cancer diagnosed worldwide annually, resulting in around 185,000 deaths from the disease. Epithelial ovarian cancer comprises approximately 90% of all ovarian malignancies, with 50% occurring in women aged over 65 years and a 5-year survival rate for advanced disease of around 30%. Both progression free and overall survival rates for ovarian cancer reduce as patients' disease recurs, with median progression free survival (PFS) and overall survival (OS) of 5.6 and 8.9 months respectively for patients who relapse for the third time, reducing to 4.1 and 5 months respectively for the fifth relapse.

Diagnosis is usually at an advanced stage (stage III or IV) and first line treatment (induction) typically includes debulking surgery and intravenous or intraperitoneal platinum-based combination chemotherapy.

More than 80% of patients will experience disease recurrence requiring secondary treatment. For patients whose disease recurs more than 6 months after cessation of the first line platinum-based induction, re-treatment with a platinum or platinum-containing combination is recommended. For patients who progress before cessation of induction therapy (platinum refractory) or within 6 months after cessation of induction therapy (platinum resistant), platinum therapy is not recommended, as the likelihood of response to platinum re-exposure diminishes with decreasing interval since last platinum-based chemotherapy.

It is an object of the present application to provide an improved treatment for cancer, or otherwise to obviate and/or mitigate issues with existing treatments.

### SUMMARY

According to a first aspect of the present application, there is provided a cyclodextrin-containing polymer-topoisomerase inhibitor conjugate for use in treating ovarian cancer in a subject;
wherein the use is in combination with a poly (ADP-ribose) polymerase (PARP) inhibitor; and
wherein the subject has previously undergone chemotherapy comprising a platinum-based chemotherapeutic agent prior to said use.

According to a second aspect of the present application, there is provided a method for treating ovarian cancer in a subject, the method comprising:
administering to a subject in need thereof a cyclodextrin-containing polymer-topoisomerase inhibitor conjugate;
in combination with a poly (ADP-ribose) polymerase (PARP) inhibitor;
wherein the subject has previously undergone chemotherapy comprising a platinum-based chemotherapeutic agent.

According to a third aspect of the present application, there is provided a cyclodextrin-containing polymer-topoisomerase inhibitor conjugate for use in treating cancer in a subject;
wherein the use is in combination with a poly (ADP-ribose) polymerase inhibitor; and
   wherein the cancer is selected from gastric, colorectal, cervical, and pancreatic.

According to a fourth aspect of the present application, there is provided a method for treating cancer in a subject, the method comprising:
administering to a subject in need thereof a cyclodextrin-containing polymer-topoisomerase inhibitor conjugate;
in combination with a poly (ADP-ribose) polymerase (PARP) inhibitor;
wherein the cancer is selected from gastric, colorectal, cervical, and pancreatic.

### Definitions

The section headings used herein are for organizational purposes only and are not to be construed as limiting the subject matter described.

Unless otherwise specified and/or defined herein, all technical and scientific terms herein have the meaning commonly understood to one of ordinary skill in the art and relevant technical field. For example, unless otherwise specified and/or defined herein, all technical and scientific terms herein in the field of cancer have a meaning in accordance with the NCI Dictionary of Cancer terms (as of August 2020). Additionally, unless otherwise specified and/or defined herein, all technical and scientific terms herein in the field of chemistry have a meaning in accordance with the IUPAC Gold Book (as of August 2020). Definitions herein prevail in the event there is any inconsistency.

Any methods, instruments, devices and materials similar or equivalent to those described herein can be used in the practice of this application. The following definitions are provided to facilitate understanding of certain terms used frequently herein and are not meant to limit the scope of the present disclosure.

All patents, published patent applications and publications referenced herein are hereby incorporated by reference in their entirety, unless otherwise specified.

Singular forms “a”, “an”, and “the” include plural reference unless the context dictates otherwise.

The terms “comprise”, “include” and “have” (as well as derivatives thereof), are used herein interchangeably as open-ended terms. In other words, use of these terms means that other non-specified elements, steps, or ingredients may be present.

The term “consist” excludes any element, step, or ingredient not specified.

The term “about” as applied to a specified value both includes the specified value and a range around the specified value which a person of ordinary skill in the art would consider reasonably similar to the specified value, given the content. Suitably, the term “about” should be understood to mean within a standard deviation (using measurements generally acceptable in the art). Suitably, “about” means a range+/−10% of the specified value (such as a range+/−10% of the specified value). In some instances, about means the specified value (only). For example, “about 9 months” is intended to include 9 months as well as 8.1 to 9.9 months, such as 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, and 9.9 months. For example, “about 48 hours is intended to include 48 hours, as well as 43.2 to 52.8 hours, such as 43.2, 44.2, 45.2, 46.2, 47.2, 48.2, 49.2, 50.2, 51.2, 52.2 and 52.8 hours. For example, “about 10 mg/m²” is intended to include 10 mg/m² as well as 9 to 11 mg/m², such as 9.0, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9 and 11 mg/m².

As used herein, the term “subject” (and/or “patient”) refers to an animal and is intended to include human and non-human animals. Suitably, the subject is a mammal, such as a primate (e.g., a human, monkey, chimpanzee, etc.). Preferably, the subject is a human. Alternatively, the subject is a non-primate (e.g., a dog, cat, rat, rabbit, guinea pig, mouse, camel, donkey, zebra, cow, pig, horse, goat, sheep, mouse, etc.). The term “non-human animal” includes all vertebrates, e.g., non-mammals (such as chickens, amphibians, reptiles, etc.) and mammals, such as non-human primates, domesticated and/or agriculturally useful animals, e.g., sheep, dog, cat, cow, pig, etc.

A subject or patient may be between 0 and 90 years old, such as greater than 10 years old, optionally greater than 18 years old, greater than 20 years old, greater than 30 years old, greater than 40 years old, greater than 50 years old, greater than 60 years old, greater than 70 years old, or greater than 80 years old.

The term “cancer” refers to a cellular disorder characterised by uncontrolled or dysregulated cell proliferation, decreased cellular differentiation, inappropriate ability to invade surrounding tissue, and/or ability to establish new growth at ectopic sites. The term “cancer” includes solid tumors and hematological tumors. The term “cancer” encompasses diseases of skin, tissues, organs, bone, cartilage, blood, and vessels. The term “cancer” further encompasses primary and metastatic cancers.

The present application relates to the treatment of advanced (including stage II, stage III and high grade serous) and metastatic ovarian cancer (i.e. stage IV cancer). Ovarian cancer includes (but is not limited to) epithelial ovarian carcinoma, fallopian tube cancer, germ cell cancer (e.g., a teratoma), sex cord-stromal tumor (e.g., estrogen-producing granulose cell tumor, virilizing Sertoli-Leydig tumor, arrhenoblastoma), e.g., locally advanced or metastatic ovarian cancer, childhood ovarian cancer; ovarian low malignant potential tumor, high-grade serous or endometrioid ovarian cancer, primary peritoneal cancer, fallopian-tube cancer (or a combination thereof), etc.

The terms “treat”, “treating”, “treatment”, “therapy” and the like are all intended to refer to a stabilization, amelioration or reversal of at least one measurable physical parameter (e.g. symptom) related to the disease, disorder, or condition (e.g. ovarian cancer). These terms also refer to causing regression, preventing the progression (e.g. maintaining the current state), or at least slowing down the progression of the cancer. Suitably, these terms refer to an alleviation or prevention of the development or onset, or reduction in the duration, of one or more physical parameter associated with cancer. Prevention of the development or onset, or reduction in the duration may be measured using statistical analysis to determine deviations from a control. Amelioration is achieved with the eradication or reduction of one or more of the physical parameters associated with the underlying disease, disorder, or condition such that an improvement is observed in the subject, notwithstanding that the subject may still be afflicted with the underlying disease, disorder, or condition. Suitably, these terms refer to prevention of the recurrence of the disease, disorder, or condition. For example, the terms can refer to an increase in the survival of a subject having the disease, disorder, or condition. Suitably, these terms refer to elimination of the disease, disorder, or condition in the subject.

The term “primary therapy” refers to the initial treatment given to a patient based upon the diagnosis of the disease in the patient. Primary therapy is given for the first occurrence of that disease in the patient, i.e. a newly diagnosed patient. When used by itself, primary therapy is typically that which is accepted as the most effective treatment.

“First-line therapy” or “front-line therapy” refer to the initial treatment of a newly diagnosed patient in the advanced/metastatic setting. Typical first-line therapies for advanced/metastatic ovarian cancer include paclitaxel/cisplatin; paclitaxel/carboplatin; docetaxel/carboplatin; etc. combinations. Further therapies are outlined in the National Comprehensive Cancer Network (NCCN) Clinical Practice Guidelines in Oncology (Ovarian Cancer, Version 1.2020-11 March, 2020).

The term “maintenance therapy” refers to a therapeutic regimen that is designed to prevent or delay a relapse in patients which have already undergone an initial/primary therapy and have achieved a response (complete or partial) in accordance with RECIST version 1.1. As above, preventing or delaying a relapse in patients may be measured using statistical analysis to determine deviations from a control. For example, maintenance chemotherapy may be given to people who have a cancer in remission (i.e. the cancer is responding to treatment, whether a partial response or complete response) in an effort to prevent or delay a relapse, to reduce the likelihood of disease recurrence or progression. Maintenance therapy can be provided for any length of time, including extended time periods up to the life-span of the subject. Maintenance therapy can be provided after initial/primary therapy (e.g. first line therapy) or in conjunction with initial/primary or additional therapies. A maintenance therapy does not constitute a further line of therapy. As outlined in the NCCN Ovarian Cancer guidelines, for example bevacizumab can be used as a maintenance therapy following a first-line therapy, until progression and/or intolerable toxicity.

The term “cycle” (e.g. “treatment cycle”) refers to a dosage regimen within a given line of therapy. A line of therapy comprises one or multiple cycles and each cycle comprises one or multiple administrations of a therapeutic agent (or agents) according to a specific regimen. A line of therapy may, for example, comprise multiple 28-day cycles (e.g. six 28-day cycles) and each cycle may involve a regimen comprising multiple administrations of a therapeutic agent (or agents), on specific days over the 28 cycle.

The terms “respond”, “responsive”, “responding” and the like; and “progression”, “progress”, “progressing” (such as “progressive disease (PD)”) and the like within a given line of therapy are as defined in Response Evaluation Criteria in Solid Tumors (RECIST) version 1.1 (European Journal of Cancer 45 (2009) 228-247; Eisenhauera et al.), the entire contents of which are hereby incorporated by reference.

Progression refers to an increase of at least 20% in the sum of diameters of target lesions using a baseline sum of diameters as reference (together with an absolute increase of at least 5 mm), or the appearance of a new lesion, as defined in RECIST version 1.1.

The term “objective response” refers to a measurable response and includes complete response (CR) or partial response (PR). The term “complete response” or “complete remission” refers to the disappearance of all target lesions in response to a treatment. This does not necessarily mean the cancer has been “cured”. The term “partial response” refers to a decrease in the size of one or more tumors or lesions, or in the extent of cancer in the body, in response to treatment. Specifically, a PR refers to a reduction of at least 30% in the sum of diameters of target lesions using a baseline sum of diameters as reference, as defined in RECIST version 1.1. A CR refers to the disappearance of all target lesions.

A stable disease (SD) demonstrates neither sufficient reduction to qualify for PR, nor sufficient increase to qualify for PD. Specifically, an SD refers to a reduction of less than 30% in the sum of diameters of target lesions or an increase of less than 20% in the sum of diameters of target lesions, as defined in RECIST version 1.1.

Some cancers initially respond to a given line of therapy, but lesions may grow again, or new lesions may appear, in the future. If a cancer is completely cured but then returns, this is known as “relapse” or “recurrence”.

In the context of the present application, side effects are referred to treatment related treatment emergent adverse events (TRTEAEs). The severity of all TRTEAEs are graded according to the NCI-Common Terminology Criteria for Adverse Events (CTCAE) Version 5.0 (the entire contents of which are hereby incorporated by reference) and a severe TRTEAE/side effect has grade 3 or above.

The term “line of therapy” or “line of chemotherapy” refers to the combination of all administrations, across all cycles within a given treatment decision (e.g. one or more therapies overall being selected to provide a curative therapeutic intent or maintenance therapeutic intent, based on a diagnosis of the condition of the patient). The term “initial therapy” refers to the first therapy in the treatment of cancer (e.g. after initial diagnosis). The first-line therapy is the first treatment given in the advanced/metastatic setting. If a patient progresses after a first-line therapy or if the first-line therapy produces side effects which are severe, a second-line therapy may be provided, followed by a third-line therapy if that patient progresses again (or has side effects which are severe), and so on. If first-line therapy completely or partially resolves the cancer (PR or CR), a maintenance therapy may be employed to prevent or delay relapse, or to reduce the likelihood of disease recurrence or progression. Maintenance therapy does not constitute a further line of therapy. Alternatively, if first-line therapy fails to elicit a lasting response (CR, PR or SD) and patients eventually have progressive disease (PD), or if there is no response (i.e. PD) within the RECIST version 1.1 criteria, or it produces severe side effects, then an alternative chemotherapeutic agent or combination of agents may be provided to the patient with the intention of eliciting a response, as a second-line therapy. A first-line therapy suitably comprises debulking surgery followed by therapy with a platinum-based chemotherapeutic agent.

A change between different lines of therapy may be triggered by communication between the patient and doctor. For example, the patient may indicate that side effects have become severe and thus a new therapeutic decision/line of therapy is required. Alternatively, measurements of cancer progression may indicate that a line of therapy is ineffective and a thus a new therapeutic decision/line of therapy is required.

The term “chemotherapeutic agent” means any agent that can be used to treat cancer, and includes, but is not limited to, DNA-damaging agents, DNA damage repair pathway inhibitors, anti-angiogenic agents, cytotoxic agents, cytostatic agents, debulking agents, targeted anti-cancer agents and cancer vaccines.

A patient having “previously undergone chemotherapy comprising a platinum-based chemotherapeutic agent prior to said use” in the context of the present application means that the patient has previously been administered a platinum-based chemotherapeutic agent (e.g. carboplatin, oxaliplatin and/or cisplatin) to treat the same condition now treated by the instant disclosure. Typically, the platinum-based chemotherapeutic agent is administered together with one or more further agents in a given line of therapy (e.g. in a first-line therapy). The previous administration of platinum-based chemotherapeutic agent would have been intended to resolve the disease, disorder, or condition (e.g. the ovarian cancer) or at least ameliorate or reverse at least one measurable physical parameter related to the disease, disorder, or condition which is now undergoing treatment with the combined cyclodextrin-containing polymer-topoisomerase inhibitor conjugate and poly (ADP-ribose) polymerase inhibitor therapy. In other words, the patient is now undergoing treatment for the same disease, disorder, or condition for which the platinum-based chemotherapeutic agent was previously prescribed. Suitably, the platinum-based chemotherapeutic agent may have been administered within about 5 years, such as within about 4 years, such as within about 3 years, such as within about 2 years, such as within about 1 year, such as within about 6 months of commencement of the combined therapy which is the subject of the present application.

The patient may, for example, have undergone a platinum-based first-line chemotherapy for the treatment of cancer, but that cancer may have returned (i.e. the patient may have relapsed). Alternatively, the patient may have undergone a platinum-based first-line therapy for the treatment of cancer and this may have failed to deliver the intended regression of the cancer (i.e. the cancer may be stable, refractory or resistant to the platinum-based chemotherapeutic agent). The patient may be platinum resistant or platinum refractory.

The platinum-based chemotherapeutic agent which forms part of the previous therapy has therefore been administered at a prior time, in a different line of therapy, to the combined cyclodextrin-containing polymer-topoisomerase inhibitor conjugate PARP inhibitor therapy.

A “platinum-based chemotherapeutic agent” is also referred to as a platinum-containing compound. Platinum containing compounds include cisplatin, carboplatin, oxaliplatin, satraplatin, picoplatin, nedaplatin, triplatin, for example. Platinum containing compounds typically employed in the treatment of ovarian cancer include carboplatin, oxaliplatin and cisplatin. Generally speaking, carboplatin is a preferred platinum-based chemotherapeutic agent for a first-line therapy. Oxaliplatin may be used in recurrent ovarian cancer, while cisplatin may be used in the treatment of platinum-sensitive ovarian cancer.

Without wishing to be bound by theory, it is understood that platinum-containing chemotherapeutic agents are able to cause crosslinking of DNA (e.g. as monoadducts, inter-strand crosslinks, intra-strand crosslinks or DNA protein crosslinks). In cancer cells, crosslinked DNA inhibits DNA repair and/or synthesis. Cisplatin was the first platinum-containing compound to be discovered and was first approved by the US Food and Drug Administration (FDA) in 1978. Carboplatin was introduced in the 1980s and typically gives rise to fewer side-effects than cisplatin in the treatment of ovarian cancer.

The patient may have received more than one line of therapy comprising a platinum-based chemotherapeutic agent prior to undertaking a treatment according to the present application. For example, the patient may have undergone 2, 3, 4, 5 or more lines of such therapy prior to undertaking a treatment according to the present application.

The term “platinum refractory” cancer means that the cancer has progressed during treatment with a platinum-based chemotherapy, suitably as measured at about 3 months (e.g. about 1 to 3 months) from beginning treatment with the platinum-based chemotherapy (from the first dose of the platinum-based chemotherapy).

The term “platinum resistant” cancer means that the cancer has progressed after receiving platinum-based chemotherapy, suitably as measured within about 6 months (e.g. about 1 to 6 months) from the final dose of the platinum-based chemotherapy.

A “platinum sensitive” cancer means that the cancer has not progressed after receiving platinum-based chemotherapy, suitably as measured within about 12 months (e.g. about 7 to 12 months) from the final dose of the platinum-based chemotherapy. The term “partially platinum sensitive” cancer means that the cancer patient has not progressed after receiving platinum-based chemotherapy within about 6 months (from the final dose of the platinum-based chemotherapy). In other words, a platinum sensitive or partially platinum sensitive patient is not platinum refractory or platinum resistant.

The term “survival” refers to a patient remaining alive and includes progression-free survival (PFS) and overall survival (OS). Suitably, survival is estimated by the Kaplan-Meier (KM) method. Any differences in survival may be computed using the stratified log-rank test.

The term “progression-free survival (PFS)” refers to the time from treatment (or randomization) to first disease progression or death, whichever is sooner. It may, for example, be the time that the patient remains alive, without return of cancer from initiation of treatment or from initial diagnosis. Suitably, progression-free survival is assessed by RECIST version 1.1.

The term “overall survival” refers to the patient remaining alive for a defined period of time from initiation of treatment or from initial diagnosis (irrespective of progression).

The term “naïve” (e.g. PARP-inhibitor naïve) means that the cancer patient has not previously received a particular therapy (e.g. the cancer patient has not previously received a PARP inhibitor-based therapy).

The term “concurrent”, “simultaneous”, “simultaneously” and the like, as applied to the administration of a therapy within the meaning of the present application is meant the administration of at least 2 active ingredients by the same route and at the same time or at substantially the same time.

The term “separate”, “separately” and the like, as applied to the administration of a therapy within the meaning of the present application is meant the administration of at least 2 active ingredients at the same time or at substantially the same time by different routes.

The term “substantially the same time” as used herein suitably refers to administration within the same day.

The term “sequential”, “sequentially” and the like, as applied to the administration of a therapy within the meaning of the present application is meant the administration of at least 2 active ingredients at different times, the administration routes for the at least 2 active ingredients being identical or different. More particularly by an administration method is meant according to which the whole administration of one of the active ingredients is carried out before administration of the other or others commences. It is thus possible to administer one of the active ingredients over several months before administering the other active ingredient or ingredients. There is no simultaneous treatment in this case. An alternate administration of each active ingredient over several weeks can also be envisaged.

The term “body surface area” (BSA; typically measured in m²) refers to a calculated surface area of an animal (e.g. human) body. Dosages of certain therapeutic agents herein (e.g. the cyclodextrin-containing polymer-topoisomerase inhibitor conjugate) may be based on a certain weight amount of the therapeutic agent per unit of body surface area. This is suitably calculated using the Du Bois formula:

BSA=0.007184*W^{0.425}*H^{0.725}

Where BSA is the body surface area, W is the subject's weight in kilograms and H is the height in centimeters.

Topoisomerase inhibitors are a class of chemical compounds which inhibit action of topoisomerase enzymes, such as topoisomerase I and II.

A poly (ADP-ribose) polymerase (PARP) inhibitor is a class of chemical compounds which inhibit action of poly ADP ribose polymerase (PARP) enzymes, such as PARP1, PARP2 etc.

The term “carrier” refers to any excipient, buffer, diluent, filler, stabilizer, solubilizer, oil, lipid microsphere, or lipid vesicle, liposome, or other material well known in the art suitable for use in pharmaceutical formulations.

The term “excipient” refers to a substance that aids the administration of an active agent to a subject. Pharmaceutical excipients include, but are not limited to, binders, fillers, disintegrants, lubricants, coatings, sweeteners, flavors and colors. One of skill in the art will recognize that other excipients can be useful. It will be understood that the characteristics of the carrier or excipient will depend on the route of administration for a particular application.

The term “pharmaceutically acceptable carrier” refers to a material that is compatible with a recipient subject, e.g. a non-toxic material. The pharmaceutically acceptable carrier may be suitable for delivering an active agent to the target site without terminating the activity of the agent. The toxicity or adverse effects, if any, associated with the carrier preferably are commensurate with a reasonable risk benefit ratio for the intended use of the active agent. Suitably, the carrier has is included in the Inactive Ingredient Guide prepared by the U.S. and Drug administration.

The term “salt” refers to an acid or base salt of a compound described herein. Pharmaceutically acceptable salts can be derived, for example, from mineral acids (hydrochloric acid, hydrobromic acid, phosphoric acid, and the like), organic acids (acetic acid, propionic acid, glutamic acid, citric acid and the like), and quaternary ammonium ions. It is understood that the pharmaceutically acceptable salts are substantially non-toxic.

The terms “effective amount”, “an amount effective”, “therapeutically effective amount” and the like refer to an amount of an active agent, ingredient or component that elicits a desired biological or medicinal response in a subject. A therapeutically effective amount can be determined empirically and is a matter of routine, in based on the stated purpose. In vitro assays can be employed to inform dosage ranges. Selection of an effective dose can also be informed (e.g. as determined via clinical trials) by those skilled in the art based upon the consideration of, for example, the disease to be treated or prevented, the symptoms involved, the subject's body surface area and/or the subject's body mass, age, sex and other factors known by the skilled artisan. The precise dose to be employed in the formulation will also depend on the route of administration, and the severity of disease, and should be decided according to the judgment of the practitioner and each subject's circumstances. Effective doses can be extrapolated from dose-response curves derived from in vitro or animal model test systems. An example of an “effective amount” is an amount sufficient to contribute to the treatment, or reduction of a symptom or symptoms of a disease, disorder, or condition.

The term “alkyl” refers to a substituted or unsubstituted, straight or branched hydrocarbon chain that comprises a fully saturated hydrocarbon group. The alkyl group may have 1 to 20 carbon atoms. The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. Typical alkyl groups include, but are not limited to, methyl, ethyl, propyl, isopropyl, butyl, isobutyl, tertiary butyl, pentyl and hexyl.

The term “alkenyl” refers to an alkyl group as defined above that contains in the straight or branched hydrocarbon chain one or more double bonds.

The term “alkenyl” refers to an alkyl group as defined above that contains in the straight or branched hydrocarbon chain one or more triple bonds.

The term “alkoxy” refers to the formula —OR wherein R is substituted or unsubstituted alkyl, alkenyl, alkynyl, cycloalkyl, cycloalkenyl, aryl, heteroaryl, heterocyclyl, cycloalkyl(alkyl), aryl(alkyl), heteroaryl(alkyl) or heterocyclyl(alkyl) is defined above. For example, alkoxy includes (but is not limited to) methoxy, ethoxy, n-propoxy, isopropoxy, n-butoxy, iso-butoxy, sec-butoxy, tert-butoxy, phenoxy and benzoxy.

The term “aryl” refers to a substituted or unsubstituted carbocyclic mono- or multi-cyclic aromatic ring system (including fused ring systems). The number of carbon atoms in an aryl group can vary, such as C₆-C₁₄, C₆-C₁₀, or C₆. Examples of aryl groups include, but are not limited to, benzene, naphthalene and azulene.

The term “heteroaryl” refers to a substituted or unsubstituted mono- or multi-cyclic aromatic ring system having one or more heteroatoms (for example, 1 to 5 heteroatoms). Heteroatoms are elements other than carbon, such as nitrogen, oxygen and sulfur. The number of atoms in the ring(s) of a heteroaryl group can vary. The heteroaryl group can contain 4 to 14 atoms in the ring(s), 5 to 10 atoms in the ring(s) or 5 to 6 atoms in the ring(s). “Heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, and the like.

The term “halogen” refers to any atom in group 17 of the periodic table, such as fluorine (F), chlorine (Cl), bromine (Br), iodine (I), etc.

The term “acyl” refers to a hydrogen, deuterium, alkyl, alkenyl, alkynyl, aryl or heteroaryl connected via a carbonyl group.

Whenever a group is described as being “optionally substituted”, that group is unsubstituted or substituted with one or more of the indicated substituents. Suitable groups are individually and independently selectable from alkyl, alkenyl, alkynyl, cycloalkyl, cycloalkenyl, aryl, heteroaryl, heterocyclyl, aryl(alkyl), heteroaryl(alkyl), heterocyclyl(alkyl), deuterium, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, hydroxyalkyl, haloalkyl, haloalkoxy, trihalomethanesulfonyl, trihalomethanesulfonamido, an amino, a mono-substituted amino group and a di-substituted amino group.

The terms “protecting group” and “protecting groups” and the like refer to any atom or group of atoms (i.e. moiety) that is attached to the remainder of a molecule in order to prevent the molecule from undergoing unwanted chemical reactions. Suitable protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups.

Dosages of the cyclodextrin-containing polymer-topoisomerase inhibitor conjugate as described herein are expressed in mg of camptothecin, as opposed to mg of conjugate. Here it will be understand that there may be multiple camptothecin units conjugated to the remainder of a given cyclodextrin-containing polymer-topoisomerase inhibitor conjugate. For example, CRLX-101 comprises two camptothecin units conjugated to the remainder of the cyclodextrin-containing polymer-topoisomerase inhibitor conjugate.

The terms “nanoparticle”, “nanoparticulate” and the like are particles sized about 1-1,000 nm, e.g. from about 10 to 300 nm in diameter, e.g., about 20 to 280, about 30 to 250, about 40 to 200, about 20 to 150, about 30 to 100, about 20 to 80, about 30 to 70, about 40 to 60 or about 40 to 50 nm diameter. The particle may be about 50 to 60 nm, about 20 to 60 nm, about 30 to 60 nm, about 35 to 55 nm, about 35 to 50 nm or about 35 to 45 nm in diameter. Preferably, the nanoparticle is about 10 to 50 nm. Nanoparticles may be approximately spherical or sphere-like in shape. Nanoparticle size is suitably a number average and determined using dynamic light scattering (e.g. with a Malvern Zetasizer Nano ZS90), such as in accordance with ISO 22412:2017. Polydispersity may be measured using the same technique and equipment.

Molecular weight is suitably a number average and determined by gel permeation chromatography (“GPC”) with refractive index detection, such as in accordance with ISO 16014-1:2019.

Zeta potential may suitably be measured using Malvern Zetasizer Nano ZS90, such as in accordance with ISO 13099:2012.

Olaparib (lynparza) is 4-[3-(4-cyclopropanecarbonyl-piperazine-1-carbonyl)-4-fluoro-benzyl]-2H-phthalazin-1-one. Olaparib has the structure:

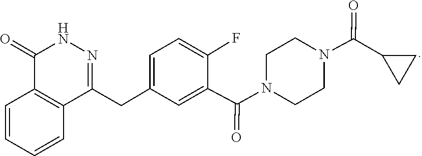 [SMILES: O=C(c1cc(Cc2n[nH]c(=O)c3ccccc23)ccc1F)N1CCN(C(=O)C2CC2)CC1]

Veliparib (ABT-888) is 2-[(R)-2-methylpyrrolidin-2-yl]-1H-benzimidazole-4-carboxamide. Veliparib has the structure:

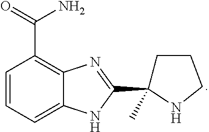 [SMILES: C[C@]1(c2nc3c(C(N)=O)cccc3[nH]2)CCCN1]

Niraparib (Zejula) is 2-{4-[(3S)-piperidin-3-yl]phenyl}-2H-indazole-7-carboxamide. Niraparib has the structure:

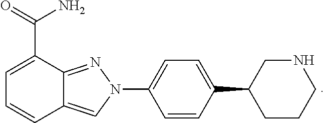 [SMILES: NC(=O)c1cccc2cn(-c3ccc([C@@H]4CCCNC4)cc3)nc12]

Rucaparib (Rubraca) is 6-fluoro-2-[4-(methylaminomethyl)phenyl]-3,10-diazatricyclo[6.4.1.04,13]trideca-1,4,6,8(13)-tetraen-9-one. Rucaparib has the structure:

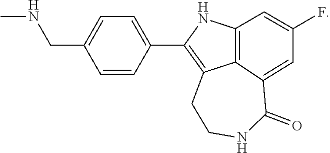 [SMILES: CNCc1ccc(-c2[nH]c3cc(F)cc4c3c2CCNC4=O)cc1]

Talazoparib (Talzenna) is (11S,12R)-7-fluoro-11-(4-fluorophenyl)-12-(2-methyl-1,2,4-triazol-3-yl)-2,3,10-triazatricyclo[7.3.1.05,13]trideca-1,5(13),6,8-tetraen-4-one. Talazoparib has the structure:

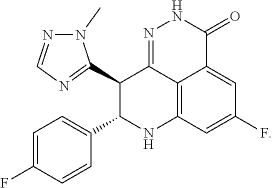 [SMILES: Cn1ncnc1[C@H]1c2n[nH]c(=O)c3cc(F)cc(c23)N[C@@H]1c1ccc(F)cc1]

Olaparib veliparib, niraparib, rucaparib and/or talazoparib may be provided as a pharmaceutically acceptable salt.

Unless otherwise defined, the following abbreviations apply:

| | |
|---|---|
| 5-FU | 5-fluorouracil |
| adm | Administered |
| ADR | Adverse drug reaction |
| AE | Adverse event |
| AUC | Area under curve |
| BID | Twice daily |
| BRCA1 | Breast cancer 1 |
| BRCA2 | Breast cancer 2 |
| BSC | Best supportive care |
| BW | Body weight |
| BWL | Body weight losses |
| C0 | Predicted concentration at time = 0 |
| CA9 | Carbonic anhydrase 9 |
| CD | Cyclodextrin |
| CDP | Cyclodextrin polyethylene glycol-based polymer |
| CI | Confidence interval |
| Cl | Clearance |
| Clobs | Observed systemic clearance |
| Cmax | Maximum concentration observed |
| CPT | 20(S)-Camptothecin |
| CrCl | Creatinine clearance |
| CRT | Chemoradiotherapy |
| CTCAE | Common Terminology Criteria for Adverse Events |
| CTCAE | Common terminology criteria for adverse events |
| CV | Coefficient of variation |
| D5W | 5% dextrose for injection |
| DLT | Dose-limiting toxicity |
| DNA | Deoxyribonucleic acid |
| EC | Ethics committee |
| ECOG | Eastern Cooperative Oncology Group |
| EPR | Enhanced permeability and retention |
| FAS | Full Analysis Set |
| FDA | Food and Drug Administration |
| GI | Gastrointestinal |
| GLP | Good Laboratory Practice |
| H2AX | phosphorylated H2A histone family, member X |
| HED | Human equivalent dose |
| HER-2 | Human epidermal growth factor receptor 2 |
| HIF | Hypoxia-inducible factor |
| HIF-1α | Hypoxia-inducible factor 1, alpha subunit |
| HIF-2α | Hypoxia-inducible factor 2, alpha subunit |
| HL | Half-life |
| HR | Hazard Ratio |
| IC50 | 50% inhibitory concentrations |
| ICPI | Interstitial Cystitis Problem Index |
| IIA | usually pilot studies designed to demonstrate |
| | clinical efficacy or biological |
| | activity (‘proof of concept’ studies) |
| IIB | find the optimum dose at which the drug |
| | shows biological activity with minimal |
| | side-effects (‘definite dose-finding’ studies). |
| IND | Investigational new drug (application) |
| IR | Infusion-related reactions |
| IRB | Institutional review board |
| IST | Investigator sponsored trial |
| ITT | Intent to treat |
| IV | Intravenous(ly) |
| KM | Kaplan-Meier |
| mCRPC | Metastatic castration resistant prostate cancer |
| MedDRA | Medical Dictionary for Regulatory Activities |
| MRT | Mean resident time |
| MTD | Maximum tolerated dose |
| NA | Not applicable |
| NC | Not calculable |
| NDC | Nanoparticle drug conjugate |
| NE | Not sufficient data for estimate |
| NOAEL | No observed adverse effect level |
| NSCLC | Non-small cell lung cancer |
| ORR | Overall response rate |
| OS | Overall survival |
| PARP | Poly ADP ribose polymerase |
| PEG | Polyethylene glycol |
| PFS | Progression-free survival |
| PK | Pharmacokinetic |
| PR | Partial regression |
| PRR | Platinum refractory/resistant |
| Q2W | Every-other-week |
| QD | Daily |
| QTc | QT interval corrected for heart rate |
| QW | Weekly |
| RBC | Red blood cell(s) |
| RP2D | Recommended phase 2 dose |
| SAE | Serious adverse event |
| SD | Standard deviation |
| SOC | Standard of care |
| SRC | Safety Review Committee |
| STD | Severely toxic dose |
| SWFI | Sterile water for injection |
| T1/2 | Terminal half-life |
| TEAE | Treatment-emergent adverse event |
| TFS | Tumour-free survivor |
| TGD | Tumour growth delay |
| Tmax | Time of maximum observed concentration |
| Topo-1 | Topoisomerase-1 |
| TPP | target product profile |
| TREA | Treatment related adverse events |
| TS | Tumor size |
| TTE | Time-to-endpoint |
| USP | United States Pharmacopeia |
| Vc | Apparent volume of the central compartment; |
| Vd | Volume of distribution |
| VHL | Von Hippel Lindau |
| Vss | Volume of distribution at steady state |
| Vssobs | Observed volume of distribution at steady state |
| WBC | White blood cell |
| XRT | Radiation therapy |

### DETAILED DESCRIPTION

According to a first aspect of the present application, there is provided a cyclodextrin-containing polymer-topoisomerase inhibitor conjugate for use in treating ovarian cancer in a subject;
wherein the use is in combination with a poly (ADP-ribose) polymerase (PARP) inhibitor; and
wherein the subject has previously undergone chemotherapy comprising a platinum-based chemotherapeutic agent prior to said use.

According to a second aspect of the present application, there is provided a method for treating ovarian cancer in a subject, the method comprising:
administering to a subject in need thereof a cyclodextrin-containing polymer-topoisomerase inhibitor conjugate;
in combination with a poly (ADP-ribose) polymerase (PARP) inhibitor;
wherein the subject has previously undergone chemotherapy comprising a platinum-based chemotherapeutic agent.

Topoisomerases are involved in regulating DNA structure. In a chemotherapeutic setting, and without wishing to be bound by theory, it is understood that topoisomerase inhibitors generate single and double stranded breaks that harm the integrity of the genome (e.g. by blocking ligation steps of the cell cycle). Introduction of these breaks subsequently leads to apoptosis and cell death.

PARP inhibitors have several mechanisms of action, including the inhibition of base excision repair (via blockade of enzymatic function) and trapping of PARP. These mechanisms lead to the induction of double-stranded breaks after stalling and collapse of the DNA replication forks. Olaparib, the first PARP inhibitor to be granted approval, is currently licensed by the EMA and FDA as monotherapy for the maintenance treatment of patients with various cancers.

Since PARP inhibitors interfere with base excision repair which is partly responsible for repair of the damage caused by chemotherapy (such as topoisomerase inhibitors as described above), addition of PARP inhibitors is able to potentiate the action of these chemotherapies when used in combination.

However, studies into similar combination therapies have uncovered significant dose-limiting hematological adverse effects, with maximum tolerated doses giving rise to sub-therapeutic effects. Overlapping toxicities of the topoisomerase inhibitor and PARP inhibitor DDRi presents a significant challenge.

One way to improve the therapeutic window of chemotherapy is to attempt to target the DNA damaging agent to the tumour and/or avoid the bone marrow compartment. Nanoparticles are designed to enhance the pharmacokinetic and pharmacodynamics properties of neoplastic agents by increasing drug solubility and enriching bio-distribution. Successful examples of nanoparticle technologies being employed include doxorubicin, paclitaxel, cytarabine, irinotecan and vincristine. Cyclodextrin-based formulation demonstrate preferential accumulation in the tumor microenvironment potentially resulting in greater concentration of chemotherapeutic action/less off-target toxicities.

Accordingly, the cyclodextrin-containing polymer-topoisomerase inhibitor conjugate may be nanoparticulate.

CRLX-101 is an investigational nanoparticle-drug conjugate, which consists of a drug delivery molecule, namely a cyclodextrin-based polymer (CDP) containing the payload camptothecin (CPT), to improve therapeutic responses in the solid tumour setting and to be able to combine more effectively with other chemotherapeutic agents or targeted therapies. In principle, its novel delivery mode allows CRLX-101, and thus the toxic anti-cancer component, to be preferentially accumulated in tumour tissue compared to normal tissues and so the toxicity/side effects are expected to be reduced, when compared to conventional chemotherapy.

Accordingly, the topoisomerase inhibitor may be a camptothecin or a camptothecin derivative, as described more fully below. Preferably, the topoisomerase inhibitor conjugate is CRLX-101 as described more fully below.

Without wishing to be bound by theory, CRLX-101 is understood to enhance antitumour activity while reducing the toxicities traditionally observed with topo-1 inhibitors, and thus may be more combinable with PARP inhibitors than other DNA damaging agents. In addition, preclinical and clinical studies have shown that CRLX-101 results in prolonged DNA damage in tumours, as set out below.

The cancer may be advanced or metastatic (preferably stage III or stage IV).

In some instances, the patient has not previously undergone chemotherapy comprising a PARP inhibitor (i.e. they may be PARP naïve). The use may be the second line of chemotherapy for treating ovarian cancer in a subject (e.g. the subject has previously undergone chemotherapy comprising the platinum-based chemotherapeutic agent as a first-line therapy). Suitably, the subject is refractory or resistant to the platinum-based chemotherapeutic agent. Preferably, therefore, the use is wherein the subject is resistant to the platinum-based chemotherapeutic agent. The use may the third or later line of chemotherapy for treating ovarian cancer in a subject (e.g. the subject has previously undergone chemotherapy comprising the platinum-based chemotherapeutic agent as a first- or second-line therapy).

The subject may, in an alternative implementation have previously undergone therapy comprising a PARP inhibitor. The therapy comprising the PARP inhibitor may be a maintenance therapy. Therapy comprising the PARP inhibitor may suitably comprise a PARP inhibitor selected from olaparib, veliparib, niraparib, rucaparib, talazoparib (preferably olaparib, niraparib or rucaparib), or a pharmaceutically acceptable salt of the foregoing.

The cancer may have progressed within about 9 months (optionally within 6 to about 9 months) after beginning the PARP-based maintenance therapy. The maintenance therapy may be the most recent therapy prior to said use. In some instances, patient has undergone PARP-based maintenance therapy for at least about 6 months, optionally at least about 9 months, optionally at least about 12 months prior to said use.

The subject may have previously undergone chemotherapy comprising a platinum-based chemotherapeutic agent with the PARP-based maintenance therapy as a next therapy after the chemotherapy comprising a platinum-based chemotherapeutic agent, said PARP-based maintenance therapy being their most recent therapy prior to said use.

The subject may be stable, refractory or resistant to the platinum-based chemotherapeutic agent. Preferably, the subject is stable or resistant to the platinum-based chemotherapeutic agent.

In certain implementations, the subject has previously undergone chemotherapy comprising a platinum-based chemotherapeutic agent and at least one other chemotherapy comprising a different chemotherapeutic agent (optionally a platinum-based chemotherapeutic agent). For example, the subject may have previously undergone chemotherapy comprising a platinum-based chemotherapeutic agent selected from carboplatin, oxaliplatin and cisplatin, optionally carboplatin.

In a specific implementation, the subject has previously undergone:
a line of chemotherapy comprising a platinum-based chemotherapeutic agent selected from carboplatin, oxaliplatin and cisplatin (optionally carboplatin); and
at least one other line of chemotherapy comprising a platinum-based chemotherapeutic agent selected from carboplatin, oxaliplatin and cisplatin (optionally oxaliplatin);
wherein the at least one other line is with the same or different platinum-based chemotherapeutic agent.

The use may be in combination with a PARP inhibitor selected from olaparib, veliparib, niraparib, rucaparib and talazoparib (preferably olaparib, niraparib or rucaparib), or a pharmaceutically acceptable salt of any of the foregoing. Olaparib, or a pharmaceutically acceptable salt thereof, is preferred for the use.

According to a third aspect of the present application, there is provided a cyclodextrin-containing polymer-topoisomerase inhibitor conjugate for use in treating cancer in a subject;
wherein the use is in combination with a poly (ADP-ribose) polymerase inhibitor; and
   wherein the cancer is selected from gastric, colorectal, cervical and pancreatic.

Preferably, the cancer is gastric cancer.

According to a fourth aspect of the present application, there is provided a method for treating cancer in a subject, the method comprising:
administering to a subject in need thereof a cyclodextrin-containing polymer-topoisomerase inhibitor conjugate;
in combination with a poly (ADP-ribose) polymerase (PARP) inhibitor;
wherein the cancer is selected from gastric, colorectal, cervical and pancreatic.

Preferably, the cancer is gastric cancer.

In an alternative implementation, the cancer can be selected from gastric, colorectal, cervical, pancreatic and myxofibrosarcoma. The cancer may be advanced or metastatic (preferably stage III or stage IV).

The cyclodextrin-containing polymer-topoisomerase inhibitor conjugate for use according to the third aspect or the method for treating cancer in a subject according to the fourth aspect (including the alternative implementation) may comprise the features of the first and second aspects outlined above.

The cyclodextrin-containing polymer-topoisomerase inhibitor conjugate for use according to the third aspect or the method for treating cancer in a subject according to the fourth aspect may further comprise combination with a further agent selected from any of the chemotherapeutic agents; hormone and/or steroids; anti-microbials; agents or procedure to mitigate potential side effects from the agent compositions such as cystitis, diarrhea, nausea and vomiting; anti-hypersensitivity agents; an agent that increases urinary excretion and/or neutralizes one or more urinary metabolite; antidiarrheal agents; antiemetic agents; immunosuppressive agents; antihistamines; anti-inflammatories; and antipyretics, such as any of those outlined under the heading “ADDITIONAL THERAPEUTIC AGENTS” below.

### Cyclodextrin-Containing Polymer-Topoisomerase Inhibitor Conjugate

The present application provides water-soluble, biocompatible polymer conjugates (e.g. cyclodextrin-containing polymer conjugates; CDP conjugates) comprising a water-soluble, biocompatible polymer covalently attached to the topoisomerase inhibitor through attachments that are cleaved under biological conditions to release the topoisomerase inhibitor.

Polymeric conjugates featured in the uses and methods described herein may be useful to improve solubility and/or stability of a bioactive/therapeutic agent, such as camptothecin, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues.

Preferably, the topoisomerase inhibitor is camptothecin or a camptothecin derivative. The term “camptothecin derivative”, as used herein, includes camptothecin analogues and metabolites of camptothecin. For example, camptothecin derivatives can have the following structures:

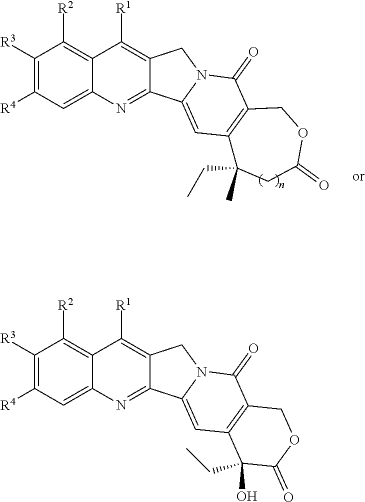

wherein

R¹ is H, OH, optionally substituted alkyl (e.g., optionally substituted with NR^{a}₂ or ORₐ, or SiR^{a}₃), or SiR^{a}₃;

R² is H, OH, NH₂, halo, nitro, optionally substituted alkyl (e.g., optionally substituted with NR^{a}₂, ORₐ, OC(═O)NR^{a}₂, or OC(═O)ORₐ);

or R¹ and R² may be taken together with the atoms to which they are attached form an optionally substituted 5- to 8-membered ring (e.g., optionally substituted with NR^{a}₂ or OR^{a}); R³ is H, OH, NH₂, halo, nitro, NR^{a}₂, OC(═O)NR^{a}₂, or OC(═O)OR^{a};

R⁴ is H, OH, NH₂, halo, CN, or NR^{a}₂;

or R³ and R⁴ may be taken together with the atoms to which they are attached form a 5- or 6-membered ring (e.g. forming a ring including —OCH₂O— or —OCH₂CH₂O—);

each R^{a} is independently H or alkyl; or two R^{a}s, taken together with the atom to which they are attached, form a 4- to 8-membered ring (e.g., optionally containing an O or NR^{b});

R_{b} is H or optionally substituted alkyl (e.g., optionally substituted with OR^{c} or NR^{c}₂);

each R^{c} is independently H or alkyl; or, two R^{c}s, taken together with the atom to which they are attached, form a 4- to 8-membered ring; and

n=0 or 1.

R¹, R², R³ and R⁴ of the camptothecin derivative may each be H, where n is 0 (if present).

R¹, R², R³ and R⁴ of the camptothecin derivative may each be H, where n is 1 (if present).

R¹ of the camptothecin derivative may be H, where R² is —CH₂N(CH₃)₂, R³ is —OH, R⁴ is H; and n is 0 (if present).

R¹ of the camptothecin derivative may be —CH₂CH₃, where R² is H, R³ is:

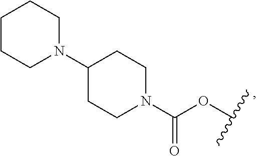 [SMILES: *OC(=O)N1CCC(N2CCCCC2)CC1]

R⁴ is H, and n is 0 (if present).

R¹ of the camptothecin derivative may be —CH₂CH₃, where R² is H, R³ is —OH, R⁴ is H, and n is 0 (if present).

R¹ of the camptothecin derivative may be tert-butyldimethylsilyl, where R² is H, R³ is —OH and R⁴ is H, and n is 0 (if present).

R¹ of the camptothecin derivative may be tert-butyldimethylsilyl, where R² is hydrogen, R³ is —OH and R⁴ is hydrogen, and n is 1 (if present).

R¹ of the camptothecin derivative may be tert-butyldimethylsilyl, where R², R³ and R⁴ are each H, and n is 0 (if present).

R¹ of the camptothecin may be tert-butyldimethylsilyl, where R², R³ and R⁴ are each H, and n is 1 (if present).

R¹ of the camptothecin derivative may be —CH₂CH₂Si(CH₃)₃ where R², R³ and R⁴ are each H (if present).

R¹ and R² of the camptothecin derivative may be taken together with the carbons to which they are attached to form an optionally substituted ring. R¹ and R² of the camptothecin derivative are taken together with the carbons to which they are attached to form a substituted 6-membered ring. The camptothecin derivative may have the following formula:

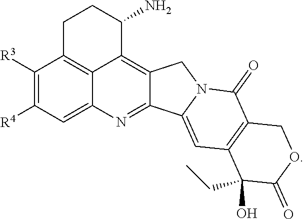

R³ may be methyl where R⁴ is fluoro.

R³ and R⁴ may be taken together with the carbons to which they are attached to form an optionally substituted ring. R³ and R⁴ may be taken together with the carbons to which they are attached to form a 6-membered heterocyclic ring.

The camptothecin derivative may have the following formula:

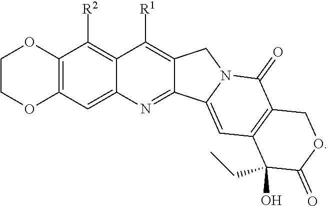

R¹ may be:

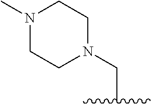

where R² is hydrogen.

The camptothecin derivative may have the following formula:

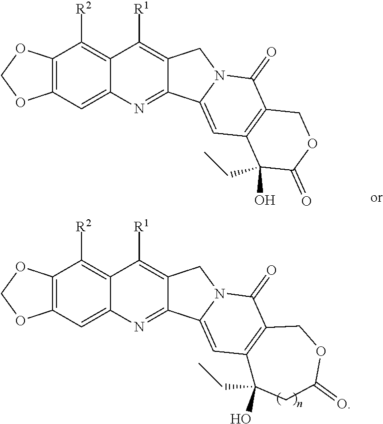

R¹ may be

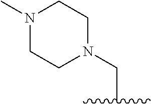

where R² is hydrogen.

R¹ may be:

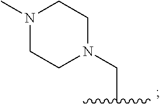

where R² is H, R³ is methyl, R⁴ is chloro; and n is 1 (if present).

R¹ may be —CH═NOC(CH₃)₃, where R², R³ and R⁴ are each H, and n is 0 (if present).

R¹ may be —CH₂CH₂NHCH(CH₃)₂, where R², R³ and R⁴ are each H; and n is 0 (if present).

R¹ and R² may be H, where R³ and R⁴ are fluoro, and n is 1 (if present).

each of R¹, R³, and R⁴ may be H, where R² is NH₂, and n is 0 (if present).

each of R¹, R³, and R⁴ may be H, where R² is NO₂, and n is 0 (if present).

Described herein are cyclodextrin containing polymer (“CDP”)-topoisomerase inhibitor conjugates, wherein one or more topoisomerase inhibitors are covalently attached to the CDP (e.g., either directly or through a linker). The CDP-topoisomerase inhibitor conjugates include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, the entire contents of which are hereby incorporated by reference.

Accordingly, the CDP-topoisomerase inhibitor conjugate may be represented by Formula I:

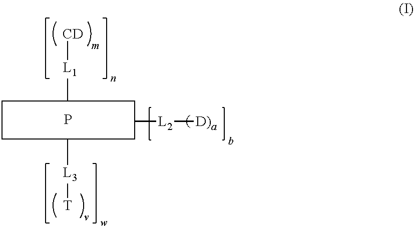

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a topoisomerase inhibitor or a prodrug thereof (e.g., a camptothecin or camptothecin derivative);

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

The CDP-topoisomerase inhibitor conjugate may be optionally substituted.

As used herein the term “targeting ligand” refers to any suitable material or substance which may promote targeting of receptors, cells, and/or tissues in vivo or in vitro with the compositions of the present invention. The targeting ligand may be synthetic, semi-synthetic, or naturally-occurring. Materials or substances which may serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, Small molecules, vitamins, steroids, steroid analogs, hormones, cofactors, bioactive agents, and genetic material, including nucleosides, nucleotides, nucleotide acid constructs and polynucleotides.

One or more of the topoisomerase inhibitor moieties in the CDP-topoisomerase inhibitor conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

P may contain a plurality of cyclodextrin moieties within the polymer chain as opposed to the cyclodextrin moieties being grafted on to groups pendant the polymeric chain. Thus, the polymer chain of formula I may further comprise n′ units of U, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U is represented by one of the general formulae below:

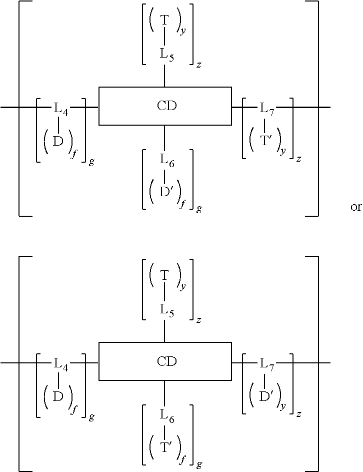

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different topoisomerase inhibitor or prodrug forms thereof (e.g., a camptothecin or camptothecin derivative);

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. Preferably, at least 50% of the U units have at least one D or D′. One or more of the topoisomerase inhibitor moieties in the CDP-topoisomerase conjugate can be replaced with another therapeutic agent, e.g., another anticancer agent or anti-inflammatory agent.

L₄ and L₇ may represent linker groups.

The CDP may include a polycation, polyanion, or non-ionic polymer. A polycationic or polyanionic polymer has at least one site that bears a positive or negative charge, respectively. Suitably, at least one of the linker moiety and the cyclic moiety comprises such a charged site, so that every occurrence of that moiety includes a charged site. The CDP is preferably biocompatible.

The CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. The polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof. Preferably, the CDP includes PEG.

The CDP-topoisomerase inhibitor conjugate may be represented by Formula II:

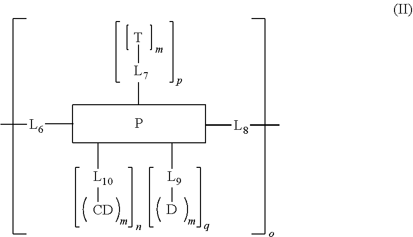

wherein

P represents a monomer unit of a polymer that comprises cyclodextrin moieties (and optionally also PEG moieties);

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a topoisomerase inhibitor or a prodrug form thereof (e.g., a camptothecin or camptothecin derivative);

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

One or more of the topoisomerase inhibitor moieties in the CDP-topoisomerase inhibitor conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

The CDP-topoisomerase inhibitor conjugate may be represented either of the formulae below:

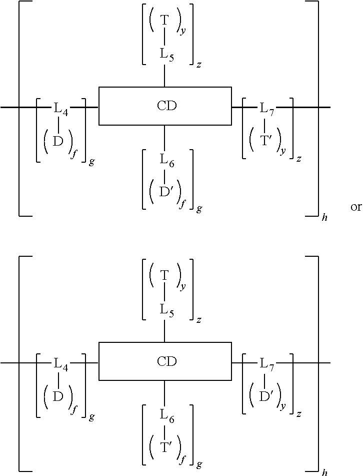

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different topoisomerase inhibitor or prodrug thereof (e.g., a camptothecin or camptothecin derivative);

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5), wherein at least one occurrence (and preferably at least 5, 10, or even at least 20, 50, or 100 occurrences) of g represents an integer greater than 0.

Preferably the polymer has a plurality of D or D′ moieties. Preferably, at least 50% of the polymer repeating units have at least one D or D′. One or more of the topoisomerase inhibitor moieties in the CDP-topoisomerase inhibitor conjugate can be replaced with another therapeutic agent, e.g., another anticancer agent or anti-inflammatory agent.

L4 and L7 preferably represent linker groups.

The CDP preferably comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6, 15-crown-5, 12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain instances, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. Conjugates may be soluble in water to a concentration of at least 0.1 g/mL, preferably at least 0.25 g/mL.

Thus, the application relates to novel compositions of therapeutic cyclodextrin-containing polymeric compounds designed for drug delivery of a topoisomerase inhibitor. These CDPs improve drug stability and/or solubility, and/or reduce toxicity, and/or improve efficacy of the topoisomerase inhibitor when used in vivo. Furthermore, by selecting from a variety of linker groups, and/or targeting ligands, the rate of topoisomerase inhibitor release from the CDP can be attenuated for controlled delivery.

The CDP may comprise a linear cyclodextrin-containing polymer, e.g., the polymer backbone may include cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. The polymer may be a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

The CDP-topoisomerase inhibitor conjugate may comprise a water soluble linear polymer conjugate comprising: cyclodextrin moieties; comonomers which do not contain cyclodextrin moieties (comonomers); and a plurality of topoisomerase inhibitors; wherein the CDP-topoisomerase inhibitor conjugate comprises at least four, five six, seven, eight, etc., cyclodextrin moieties and at least four, five six, seven, eight, etc., comonomers. The topoisomerase inhibitor may be a topoisomerase inhibitor described herein, for example, the topoisomerase inhibitor may be a camptothecin or camptothecin derivative described herein. The topoisomerase inhibitor can be attached to the CDP via a functional group such as a hydroxyl group, or where appropriate, an amino group.

One or more of the topoisomerase inhibitor moieties in the CDP-topoisomerase inhibitor conjugate can be replaced with another therapeutic agent, e.g., another anticancer agent or anti-inflammatory agent.

The least four cyclodextrin moieties and at least four comonomers may alternate in the CDP-topoisomerase inhibitor conjugate. The topoisomerase inhibitors may be cleaved from the CDP-topoisomerase inhibitor conjugate under biological conditions to release the topoisomerase inhibitor. The cyclodextrin moieties may comprise linkers to which topoisomerase inhibitors are linked. The topoisomerase inhibitors may be attached via linkers.

The comonomer may comprise residues of at least two functional groups through which reaction and linkage of the cyclodextrin monomers was achieved. In some instances, the functional groups, which may be the same or different, terminal or internal, of each comonomer comprise an amino, acid, imidazole, hydroxyl, thio, acyl halide, —HC═CH—, —C≡C— group, or derivative thereof. In some instances, the two functional groups are the same and are located at termini of the comonomer precursor. A comonomer may contain one or more pendant groups with at least one functional group through which reaction and thus linkage of a topoisomerase inhibitor was achieved. The functional groups, which may be the same or different, terminal or internal, of each comonomer pendant group may comprise an amino, acid, imidazole, hydroxyl, thiol, acyl halide, ethylene, ethyne group, or derivative thereof. The pendant group may be a substituted or unsubstituted branched, cyclic or straight chain C1-C10 alkyl, or arylalkyl optionally containing one or more heteroatoms within the chain or ring. The cyclodextrin moiety may comprise an alpha, beta, or gamma cyclodextrin moiety. The topoisomerase inhibitor may be at least 5%, 10%, 15%, 20%, 25%, 30%, or 35% by weight of CDP-topoisomerase inhibitor conjugate.

The comonomer may comprise polyethylene glycol of molecular weight from about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)), the cyclodextrin moiety comprises beta-cyclodextrin, the theoretical maximum loading of the topoisomerase inhibitor on the CDP-topoisomerase inhibitor conjugate is 13% by weight, and the topoisomerase inhibitor is 6-10% by weight of CDP-topoisomerase inhibitor conjugate. The topoisomerase inhibitor may be poorly soluble in water. The solubility of the topoisomerase inhibitor may be <5 mg/ml at physiological pH (e.g. around pH 7.4). The topoisomerase inhibitor may be a hydrophobic compound with a log P>0.4, >0.6, >0.8, >1, >2, >3, >4, or >5.

The topoisomerase inhibitor may be attached to the CDP via a second compound.

Administration of the CDP-topoisomerase inhibitor conjugate to a subject results in release of the topoisomerase inhibitor over a period of at least 6 hours. Administration of the CDP-topoisomerase inhibitor conjugate to a subject results in release of the topoisomerase inhibitor over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days, e.g. up to a month. Upon administration of the CDP-topoisomerase inhibitor conjugate to a subject, the rate of topoisomerase inhibitor release may dependent primarily upon the rate of hydrolysis as opposed to enzymatic cleavage.

The CDP-topoisomerase inhibitor conjugate may have a molecular weight of 10,000-500,000. The cyclodextrin moieties may make up at least about 2%, 5%, 10%, 20%, 30%, 50% or 80% of the CDP-topoisomerase inhibitor conjugate by weight.

The CDP-topoisomerase inhibitor conjugate may be made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. The cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

A comonomer of the CDP-topoisomerase inhibitor conjugate may comprise a moiety selected from the group consisting of: alkyl, polysuccinic anhydride, poly-L-glutamic acid, poly(ethyleneimine), an oligosaccharide, and an amino acid chain. A CDP-topoisomerase inhibitor conjugate comonomer may comprise a polyethylene glycol chain. A comonomer may comprise a moiety selected from: polyglycolic acid and polylactic acid chain. A comonomer may comprise an alkyl group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, 0 or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

The CDP-topoisomerase inhibitor conjugate may be a polymer having attached thereto a plurality of D moieties of the following formula:

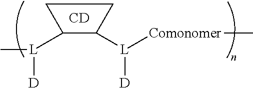

wherein each L is independently a linker, and each D is independently a topoisomerase inhibitor, a prodrug derivative thereof, e.g., a camptothecin or camptothecin derivative, or absent; and each comonomer is independently a comonomer described herein (preferably comprising PEG), and n is at least 4, 5, 6, 7, 8, 9, 10, 11, 12, 13, 14, 15, 16, 17, 18, 19 or 20, provided that the polymer comprises at least one topoisomerase inhibitor and in some instances, at least two topoisomerase inhibitor moieties. The molecular weight of the comonomer may be from about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)).

The topoisomerase inhibitor may be a topoisomerase inhibitor described herein, for example, the topoisomerase inhibitor is a camptothecin or camptothecin derivative described herein. The topoisomerase inhibitor can be attached to the CDP via a functional group such as a hydroxyl group, or where appropriate, an amino group. One or more of the topoisomerase inhibitor moieties in the CDP-topoisomerase inhibitor conjugate can be replaced with another therapeutic agent, e.g., another anticancer agent or anti-inflammatory agent.

The CDP-topoisomerase inhibitor conjugate may be a polymer having attached thereto a plurality of D moieties of the following optionally substituted formula:

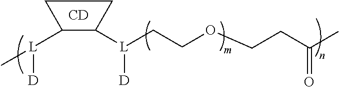

wherein each L is independently a linker, and each D is independently a topoisomerase, a prodrug derivative thereof, e.g., a camptothecin or camptothecin derivative, or absent, provided that the polymer comprises at least one topoisomerase inhibitor and in some instances, at least two topoisomerase inhibitor moieties; and

wherein the group

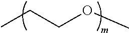

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da, preferably about 3400 Da), e.g. such that m is selected to give such a molecular weight, e.g. m may be between about 40 and 100, optionally between about 60 and 90, optionally between about 70 and 80, preferably about 75 and 80, such as about 77) and n is at least 4, 5, 6, 7, 8, 9, 10, 11, 12, 13, 14, 15, 16, 17, 18, 19 or 20. Preferably, n is between about 10 and 20, such as about 12 to 16, preferably about 14. In some instances, m may be between about 40 and 100, optionally between about 60 and 90, optionally between about 70 and 80, preferably about 75 and 80, such as about 77.

The topoisomerase inhibitor may be a topoisomerase inhibitor described herein, for example, the topoisomerase is a camptothecin or camptothecin derivative described herein. The topoisomerase inhibitor can be attached to the CDP via a functional group such as a hydroxyl group, or where appropriate, an amino group. One or more of the topoisomerase inhibitor moieties in the CDP-topoisomerase inhibitor conjugate can be replaced with another therapeutic agent, e.g., another anticancer agent or anti-inflammatory agent.

In some instances, less than all of the L moieties are attached to D moieties, meaning in some instances, at least one D is absent. The loading of the D moieties on the CDP-topoisomerase inhibitor conjugate may be from about 1 to about 50% (e.g., from about 1 to about 25%, from about 5 to about 20% or from about 5 to about 15%). Each L may independently comprise an amino acid or a derivative thereof. Each L may independently comprise a plurality of amino acids or derivatives thereof. Each L may be independently a dipeptide or derivative thereof. L may be one or more of: alanine, arginine, histidine, lysine, aspartic acid, glutamic acid, serine, threonine, asparagine, glutamine, cysteine, glycine, proline, isoleucine, leucine, methionine, phenylalanine, tryptophan, tyrosine and valine. A linker comprising cysteine and glycine units is preferred.

The CDP-topoisomerase inhibitor conjugate may be a polymer having attached thereto a plurality of L-D moieties of the following optionally substituted formula:

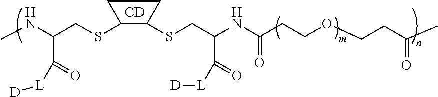

wherein each L is independently a linker or absent and each D is independently a topoisomerase inhibitor, a prodrug derivative thereof, e.g., a camptothecin or camptothecin derivative, or absent and wherein the group

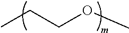

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77) and n is at least 4, 5, 6, 7, 8, 9, 10, 11, 12, 13, 14, 15, 16, 17, 18, 19 or 20, provided that the polymer comprises at least one topoisomerase inhibitor and in some instances, at least two topoisomerase inhibitor moieties.

Less than all of the C(═O) moieties may be attached to L-D moieties, meaning at least one L and/or D is absent. The loading of the L, D and/or L-D moieties on the CDP-topoisomerase inhibitor conjugate may be from about 1 to about 50% (e.g., from about 1 to about 25%, from about 5 to about 20% or from about 5 to about 15%). Each L may be independently an amino acid or derivative thereof. Each L may be glycine or a derivative thereof.

One or more of the topoisomerase inhibitor moieties in the CDP-topoisomerase inhibitor conjugate can be replaced with another therapeutic agent, e.g., another anticancer agent or anti-inflammatory agent.

The CDP-topoisomerase inhibitor conjugate may be a polymer having the following optionally substituted formula:

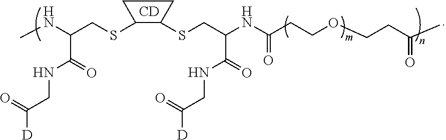

Preferably, n is between about 10 and 20, such as about 12 to 16, preferably about 14. In some instances, m may be between about 40 and 100, optionally between about 60 and 90, optionally between about 70 and 80, preferably about 75 and 80, such as about 77.

In some instances, less than all of the C(═O) moieties are attached to

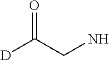

moieties, meaning

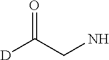

is absent in some instances, provided that the polymer comprises at least one topoisomerase inhibitor and at least two topoisomerase inhibitor moieties. In some instances, the loading of the

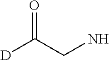

moieties on the CDP-topoisomerase inhibitor conjugate may be from about 1 to about 50% (e.g., from about 1 to about 25%, from about 5 to about 20% or from about 5 to about 15%). Preferably the group

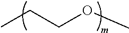

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da, preferably about 3400 Da), e.g. such that m is selected to give such a molecular weight, e.g. m may be between about 40 and 100, optionally between about 60 and 90, optionally between about 70 and 80, preferably about 75 and 80, such as about 77) and n is at least 4, 5, 6, 7, 8, 9, 10, 11, 12, 13, 14, 15, 16, 17, 18, 19 or 20.

A CDP-camptothecin conjugate described herein has a terminal amine and/or a terminal carboxylic acid. Preferably, a CDP-camptothecin conjugate described herein has a left hand (as depicted above) terminal CH₃C(O)— group and a right hand (as depicted above) terminal —NHCH₂CH(CH₃)OH group.

One or more of the topoisomerase inhibitor moieties in the CDP-topoisomerase inhibitor conjugate can be replaced with another therapeutic agent, e.g., another anticancer agent or anti-inflammatory agent.

The CDP-topoisomerase inhibitor conjugate can contain a topoisomerase inhibitor and at least one additional therapeutic agent. For instance, a topoisomerase inhibitor and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

The cyclodextrin moieties may make up at least about 2%, 5% or 10% by weight, up to 20%, 30%, 50% or even 80% of the CDP by weight. The topoisomerase inhibitors, or targeting ligands may make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, M, varies between about 12,000 and 65,000 daltons. M, may vary between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

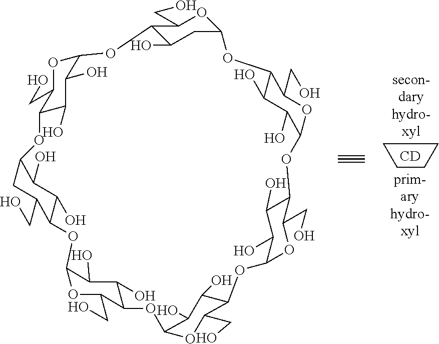

Cyclodextrins have generally toroidal, frustoconical three-dimensional shapes. The primary and secondary hydroxyl groups are oriented along the longitudinal axis of the cone and diametrically oppose one another, with the primary hydroxyl groups occupying one side if the cone/torus and the secondary hydroxyl groups occupying the opposite side. The side of the cone/torus on which the secondary hydroxyl groups are located has a wider diameter than the side on which the primary hydroxyl groups are located. The present application contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332(1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

The compounds may comprise cyclodextrin moieties and wherein at least one or a plurality of the cyclodextrin moieties of the CDP-topoisomerase inhibitor conjugate may be oxidized. The cyclodextrin moieties of P may alternate with linker moieties in the polymer chain.

In addition to a cyclodextrin moiety, the CDP can also include a comonomer, for example, a comonomer described herein. A comonomer of the CDP-topoisomerase inhibitor conjugate may comprise a moiety selected from the group consisting of: an alkyl, polysuccinic anhydride, poly-L-glutamic acid, poly(ethyleneimine), an oligosaccharide, and an amino acid chain. A CDP-topoisomerase inhibitor conjugate comonomer may comprise a polyethylene glycol chain. A comonomer may comprise a moiety selected from: polyglycolic acid and polylactic acid chain. A comonomer may comprise an alkyl group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

A comonomer can be and/or can comprise a linker such as a linker described herein.

The CDP-topoisomerase inhibitor conjugate may form a particle, e.g., a nanoparticle. The particle can comprise a CDP-topoisomerase inhibitor conjugate, e.g., a plurality of CDP-topoisomerase inhibitor conjugates, e.g., CDP-topoisomerase inhibitor conjugates having the same topoisomerase inhibitor or different topoisomerase inhibitors. The compositions described herein comprise a CDP-topoisomerase inhibitor conjugate or a plurality of CDP-topoisomerase inhibitor conjugates. The composition can also comprise a particle or a plurality of particles described herein.

The CDP-topoisomerase inhibitor conjugate containing the inclusion complex may form a particle, e.g., a nanoparticle. The nanoparticle ranges in size from 10 to 300 nm in diameter, e.g., 20 to 280, 30 to 250, 40 to 200, 20 to 150, 30 to 100, 20 to 80, 30 to 70, 40 to 60 or 40 to 50 nm diameter. The particle may be 50 to 60 nm, 20 to 60 nm, 30 to 60 nm, 35 to 55 nm, 35 to 50 nm or 35 to 45 nm in diameter. Preferably, the nanoparticle is about 10 to 50 nm.

The surface charge of the molecule may be neutral, or slightly negative. The zeta potential of the particle surface may be from about −80 mV to about 50 mV, about −20 mV to about 20 mV, about −20 mV to about −10 mV, or about −10 mV to about 0.

The CDP-topoisomerase inhibitor conjugate may be a polymer having the following optionally substituted formula C:

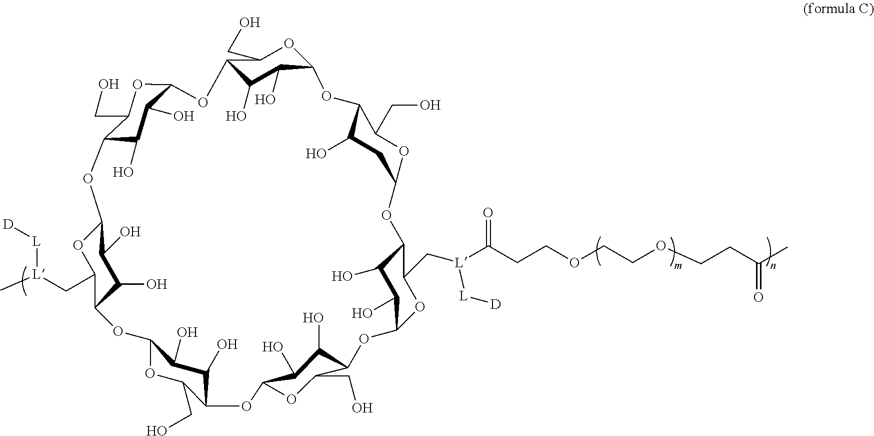

wherein L and L′ independently for each occurrence, may be a linker (e.g. L′ may be cysteine and L may be glycine), a bond, or —OH and D, independently for each occurrence, may be a topoisomerase inhibitor such as camptothecin (“CPT”), a camptothecin derivative or absent, and

wherein the group

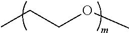

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77) and n is at least 4, provided that at least one D is CPT or a camptothecin derivative. Preferably, n is between about 10 and 20, such as about 12 to 16, preferably about 14. At least 2 D moieties are CPT and/or a camptothecin derivative for formula C.

Each L′, for each occurrence, may be a cysteine. The cysteine may be attached to the cyclodextrin via a sulfide bond. The cysteine may be attached to the PEG containing portion of the polymer via an amide bond.

L may be a linker (e.g., an amino acid, preferably glycine). L may be absent. D-L together may form

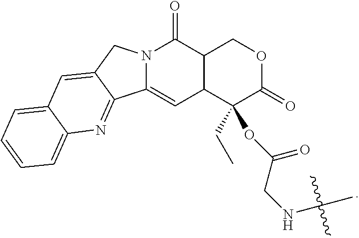 [SMILES: *NCC(=O)O[C@]1(CC)C(=O)OCC2C(=O)N3Cc4cc5ccccc5nc4C3=CC21]

A plurality of D moieties may be absent and at the same position on the polymer, the corresponding L is —OH.

Less than all of the C(═O) moieties of the cysteine residue in the polymer backbone may be attached to

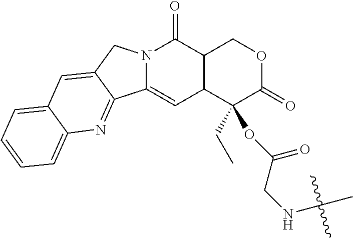 [SMILES: *NCC(=O)O[C@]1(CC)C(=O)OCC2C(=O)N3Cc4cc5ccccc5nc4C3=CC21]

moieties, meaning in some instances,

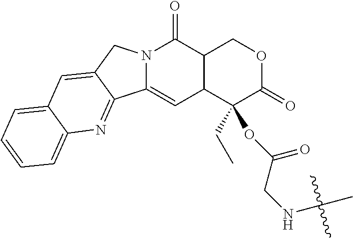 [SMILES: *NCC(=O)O[C@]1(CC)C(=O)OCC2C(=O)N3Cc4cc5ccccc5nc4C3=CC21]

is absent in one or more positions of the polymer backbone, provided that the polymer comprises at least one

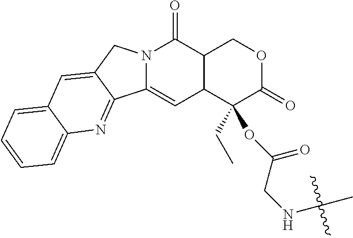 [SMILES: *NCC(=O)O[C@]1(CC)C(=O)OCC2C(=O)N3Cc4cc5ccccc5nc4C3=CC21]

and in some instances, at least two

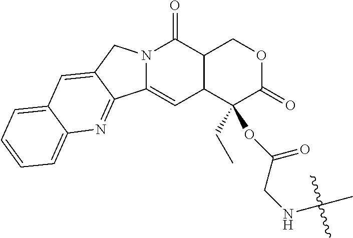 [SMILES: *NCC(=O)O[C@]1(CC)C(=O)OCC2C(=O)N3Cc4cc5ccccc5nc4C3=CC21]

moieties. In some instances, the loading of the

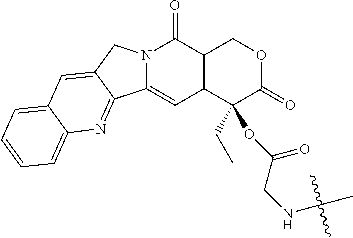 [SMILES: *NCC(=O)O[C@]1(CC)C(=O)OCC2C(=O)N3Cc4cc5ccccc5nc4C3=CC21]

moieties on the CDP-topoisomerase inhibitor conjugate may be from about 1 to about 50% (e.g., from about 1 to about 25%, from about 5 to about 20% or from about 5 to about 15%, e.g., from about 6 to about 10%). The loading of

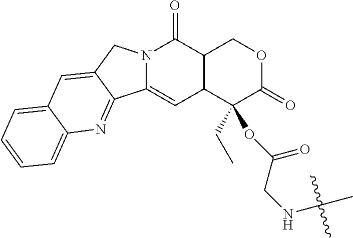 [SMILES: *NCC(=O)O[C@]1(CC)C(=O)OCC2C(=O)N3Cc4cc5ccccc5nc4C3=CC21]

on the CDP may be from about 6% to about 10% by weight of the total polymer.

The CDP-topoisomerase inhibitor conjugate of formula C may be a polymer having the following optionally substituted formula:

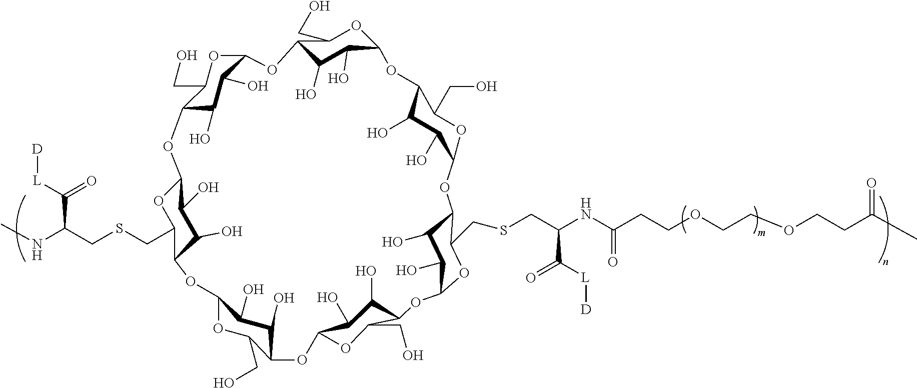

wherein L, independently for each occurrence, is a linker, a bond, or —OH and D, independently for each occurrence, is camptothecin (“CPT”), a camptothecin derivative or absent, and

wherein the group

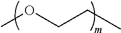

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77) and n is at least 4, provided that at least one D is CPT or a camptothecin derivative. At least 2 D moieties may be CPT and/or a camptothecin derivative. Preferably, n is between about 10 and 20, such as about 12 to 16, preferably about 14.

The CDP-camptothecin conjugate of formula C may be a polymer of the following optionally substituted formula:

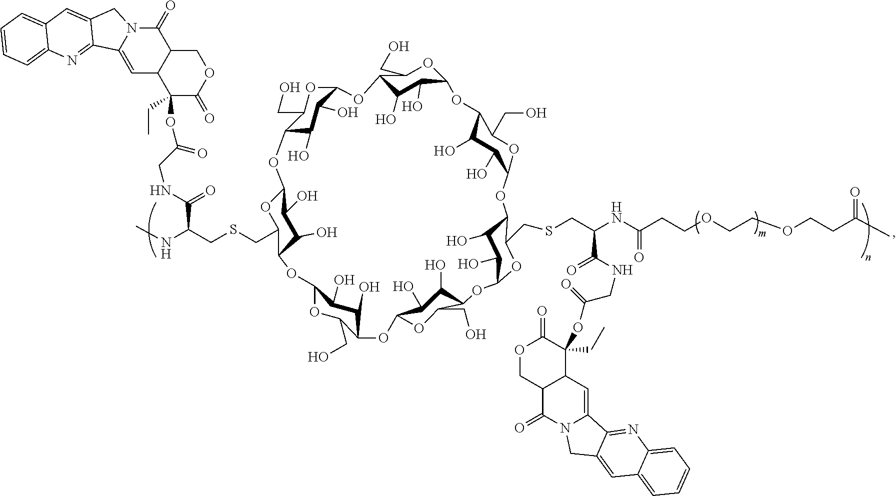

wherein m and n are as defined above. Less than all of the C(═O) sites of the cysteine of the polymer backbone may be occupied as indicated above with the CPT-Gly, but instead are free acids, meaning, the theoretical loading of the polymer (with camptothecin) is less than 100%.

In a particularly preferred implementation, the CDP-camptothecin conjugate is CRLX-101, having the following optionally substituted formula:

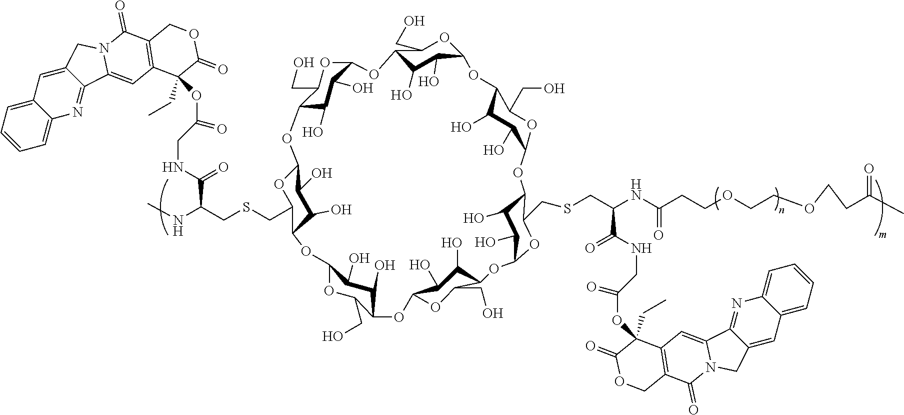

wherein

n=about 77 or the group

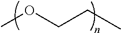

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77);

m=from about 10 to about 18 (e.g., about 14);

the molecular weight of the polymer backbone (i.e., the polymer minus the CPT-gly, which results in the cysteine moieties having a free —C(O)OH) may be from about 48 to about 85 kDa;

the polydispersity of the polymer backbone is less than about 2.2. Preferably, the loading of the CPT onto the polymer backbone is from about 6 to about 13% by weight, wherein 13% is theoretical maximum, meaning, in some instances, one or more of the cysteine residues has a free —C(O)OH (i.e., it lacks the CPT-gly). Preferably, CRLX-101 is a nanoparticle sized from about 10 to 50 nm.

The polydispersity of the PEG component in the above structure may be less than about 1.1.

The CDP-camptothecin conjugate may have the optionally substituted formula:

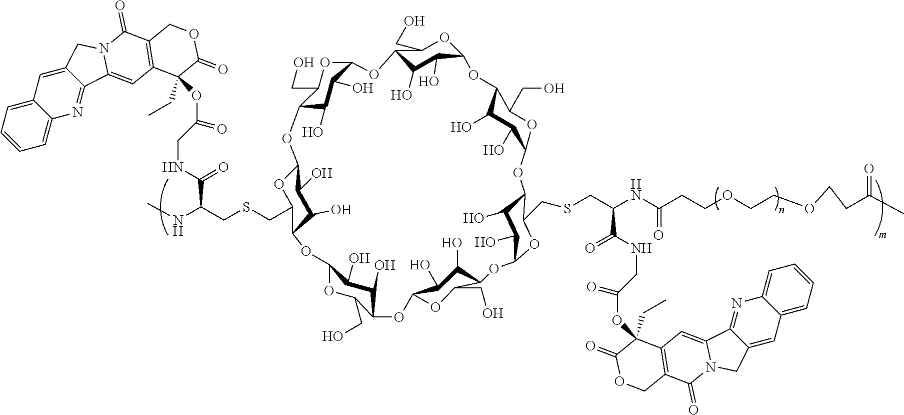

wherein m is between about 10 and 20, such as about 12 to 16, preferably about 14. In some instances, n may be between about 40 and 100, optionally between about 60 and 90, optionally between about 70 and 80, preferably about 75 and 80, such as about 77.

A CDP-camptothecin conjugate described herein has a terminal amine and/or a terminal carboxylic acid. Preferably, a CDP-camptothecin conjugate described herein has a left hand (as depicted above) terminal CH₃C(O)— group and a right hand (as depicted above) terminal —NHCH₂CH(CH₃)OH group. For example, in a preferred implementation, CRLX-101 is optionally substituted:

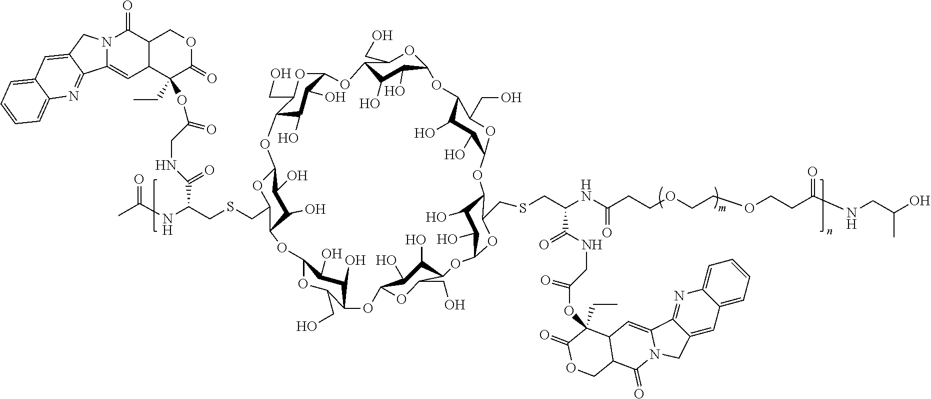

wherein n is between about 10 and 20, such as about 12 to 16, preferably about 14 and m is from about 40 to 100, optionally about 60 to 90, optionally about 70 to 80, preferably about 75 to 80, such as about 77.

The CDPs described herein can include on or more linkers. A linker can link a topoisomerase inhibitor to a CDP. A linker can link camptothecin or a camptothecin derivative to a CDP. For example, when referring to a linker that links a topoisomerase inhibitor to the CDP, the linker can be referred to as a tether.

A plurality of the linker moieties may be attached to a topoisomerase inhibitor or prodrug thereof and are cleaved under biological conditions. Preferably, the linker is an amino acid, such as a glycine linker.

Described herein are CDP-topoisomerase inhibitor conjugates comprising a CDP covalently attached to a topoisomerase inhibitor through attachments that are cleaved under biological conditions to release the topoisomerase inhibitor. A CDP-topoisomerase inhibitor conjugate may comprise a topoisomerase inhibitor covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the topoisomerase inhibitor.

Such topoisomerase inhibitors may be covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

The CDP-topoisomerase inhibitor conjugate may comprise a topoisomerase inhibitor covalently attached to the CDP through a tether, wherein the tether comprises a self-cyclizing moiety. The tether may further comprise a selectivity-determining moiety. Thus, one aspect of the application relates to a polymer conjugate comprising a topoisomerase inhibitor covalently attached to a polymer, preferably a biocompatible polymer, through a tether, wherein the tether comprises a selectivity-determining moiety and a self-cyclizing moiety which are covalently attached to one another.

The selectivity-determining moiety may be bonded to the self-cyclizing moiety between the self-cyclizing moiety and the CDP.

The selectivity-determining moiety may be a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

The application contemplates any combination of the foregoing. Those skilled in the art will recognize that, for example, any topoisomerase inhibitor of the application in combination with any linker (e.g., self-cyclizing moiety, any selectivity-determining moiety, and/or any topoisomerase inhibitor) are within the scope of the application.

The selectivity-determining moiety may be selected such that the bond is cleaved under acidic conditions.

The selectivity-determining moiety may be selected such that the bond is cleaved under basic conditions, the selectivity-determining moiety is an aminoalkylcarbonyloxyalkyl moiety. The selectivity-determining moiety may have a structure

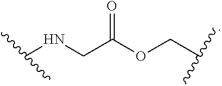 [SMILES: *COC(=O)CN*]

The selectivity-determining moiety may be selected such that the bond is cleaved enzymatically, it may be selected such that a particular enzyme or class of enzymes cleaves the bond. In preferred instances, the selectivity-determining moiety may be selected such that the bond is cleaved by a cathepsin, preferably cathepsin B.

The selectivity-determining moiety may comprise a peptide, preferably a dipeptide, tripeptide, or tetrapeptide. The peptide may be a dipeptide is selected from KF and FK, The peptide may be a tripeptide is selected from GFA, GLA, AVA, GVA, GIA, GVL, GVF, and AVF. The peptide may be a tetrapeptide selected from GFYA and GFLG, preferably GFLG.

The peptide, such as GFLG, may be selected such that the bond between the selectivity-determining moiety and the self-cyclizing moiety is cleaved by a cathepsin, preferably cathepsin B.

The selectivity-determining moiety may be represented by Formula A:

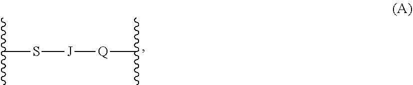

wherein

S a sulfur atom that is part of a disulfide bond;

J is optionally substituted alkenyl; and

Q is O or NR¹³, wherein R¹³ is hydrogen or alkyl.

J may be polyethylene glycol, polyethylene, polyester, alkenyl, or alkyl. J may represent an alkenyl group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. J may be substituted or unsubstituted lower alkyl, such as ethyl. For example, the selectivity-determining moiety may be

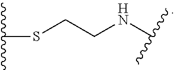 [SMILES: *NCCS*]

The selectivity-determining moiety may be represented by Formula B:

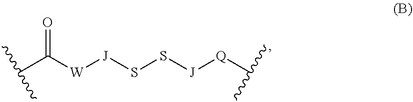

wherein

W is either a direct bond or selected from lower alkyl, NR¹⁴, S, O;

S is sulfur;

J, independently and for each occurrence, is alkenyl or polyethylene glycol;

Q is O or NR¹³, wherein R¹³ is hydrogen or alkyl; and

R¹⁴ is selected from hydrogen and alkyl.

J may be substituted or unsubstituted lower alkyl, such as methylene. J may be an optionally substituted aryl ring. The aryl ring may be an optionally substituted benzo ring. W and S may be in a 1,2-relationship on the optionally substituted aryl ring. The aryl ring may be substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NRₓSO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

The aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}, —C(O)—R^{x}, —NRₓSO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

J, independently and for each occurrence, may be polyethylene glycol, polyethylene, polyester, alkenyl, or alkyl.

Independently and for each occurrence, the linker may comprise an alkenyl group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl.

J, independently and for each occurrence, may be substituted or unsubstituted lower alkyl. J, independently and for each occurrence, may be substituted or unsubstituted ethylene.

The selectivity-determining moiety may be selected from

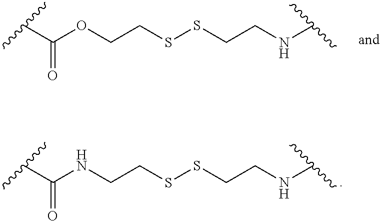 [SMILES: *NCCSSCCOC(*)=O ; *NCCSSCCNC(*)=O]

The selectivity-determining moiety may include groups with bonds that are cleavable under certain conditions, such as disulfide groups. The selectivity-determining moiety may comprise a disulfide-containing moiety, for example, comprising aryl and/or alkyl group(s) bonded to a disulfide group. The selectivity-determining moiety may have a structure

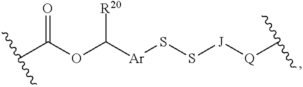

wherein

R²⁰ is an alkyl group;

Ar is a substituted or unsubstituted benzo ring;

J is optionally substituted alkenyl; and

Q is O or NR¹³

wherein R¹³ is hydrogen or alkyl.

Ar may be unsubstituted. Ar may be a 1,2-benzo ring. For example, suitable moieties within

Formula B include:

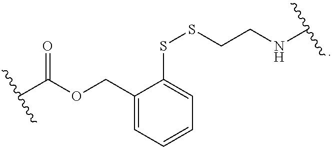 [SMILES: *NCCSSc1ccccc1COC(*)=O]

The self-cyclizing moiety may be selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

The self-cyclizing moiety may be selected such that, upon cyclization, a five- or six-membered ring is formed, preferably a five-membered ring. The five- or six-membered ring may comprise at least one heteroatom selected from oxygen, nitrogen, or sulfur, preferably at least two, wherein the heteroatoms may be the same or different. The heterocyclic ring may contain at least one nitrogen, preferably two. The self-cyclizing moiety may cyclize to form an imidazolidone.

The self-cyclizing moiety may have a structure

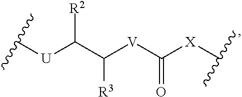

wherein

U is selected from O, NR¹ and S;

X is selected from O, NR⁵, and S, preferably O or S;

V is selected from O, S and NR⁴, preferably O or NR⁴;

R² and R³ are independently selected from hydrogen, alkyl, and alkoxy; or R² and R³ together with the carbon atoms to which they are attached form a ring; and

R¹, R⁴, and R⁵ are independently selected from hydrogen and alkyl.

U may be NR¹ and/or V may be NR⁴, where R¹ and R⁴ are independently selected from methyl, ethyl, propyl, and isopropyl. Both R¹ and R⁴ may be methyl. Both R² and R³ may be hydrogen. R² and R³ may independently be alkyl, preferably lower alkyl. R² and R³ together may be —(CH₂)ₙ— wherein n is 3 or 4, thereby forming a cyclopentyl or cyclohexyl ring. The nature of R² and R³ may affect the rate of cyclization of the self-cyclizing moiety. It would be expected that the rate of cyclization would be greater when R² and R³ together with the carbon atoms to which they are attached form a ring than the rate when R² and R³ are independently selected from hydrogen, alkyl, and alkoxy. U may be bonded to the self-cyclizing moiety.

The self-cyclizing moiety may be selected from

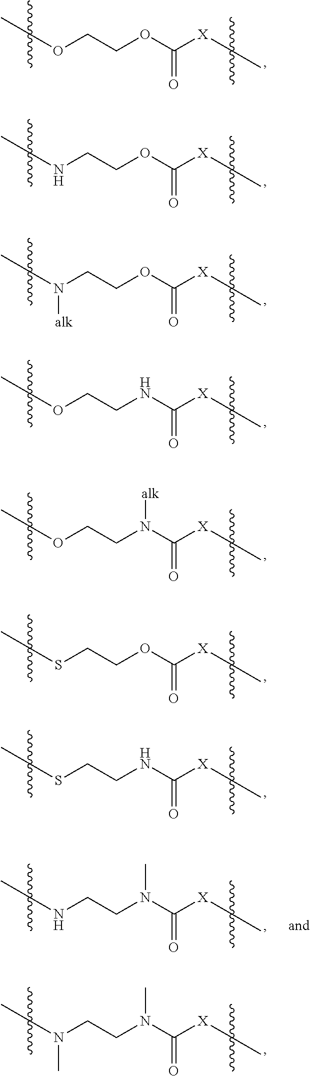

wherein “alk” is a C₁₋₆ alkyl group.

The selectivity-determining moiety may connect to the self-cyclizing moiety through carbonyl-heteroatom bonds, e.g., amide, carbamate, carbonate, ester, thioester, and urea bonds.

A topoisomerase inhibitor may be covalently attached to a polymer through a tether, wherein the tether comprises a selectivity-determining moiety and a self-cyclizing moiety which are covalently attached to one another. The self-cyclizing moiety may be selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH maybe be a self-cyclizing moiety, and ABC may be selected such that enzyme Y cleaves between C and D. Once cleavage of the bond between C and D progresses to a certain point, D will cyclize onto H, thereby releasing topoisomerase inhibitor X, or a prodrug thereof.

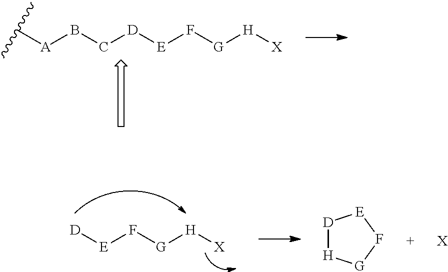

The topoisomerase inhibitor X may further comprise additional intervening components, including, but not limited to another self-cyclizing moiety or a leaving group linker, such as CO₂ or methoxymethyl, that spontaneously dissociates from the remainder of the molecule after cleavage occurs.

A linker may be and/or comprise alkylene, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. The linker group itself can be stable under physiological conditions, such as alkylene, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

The linker group(s) of the present application represent an alkenyl group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

The linker group may represent a derivatized or non-derivatized amino acid (e.g., glycine or cysteine). In certain instances, linker groups with one or more terminal carboxyl groups may be conjugated to the polymer. In certain s, one or more of these terminal carboxyl groups may be capped by covalently attaching them to a therapeutic agent, a targeting moiety, or a cyclodextrin moiety via an (thio)ester or amide bond. Linker groups with one or more terminal hydroxyl, thiol, or amino groups may be incorporated into the polymer. One or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. These (thio)ester, amide, (thio)carbonate or (thio)carbamates bonds may be biohydrolyzable, i.e., capable of being hydrolyzed under biological conditions.

A linker group may represent an alkenyl group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain instances, a linker group, e.g., between a topoisomerase inhibitor and the CDP, comprises a self-cyclizing moiety. In certain instances, a linker group, e.g., between a topoisomerase inhibitor and the CDP, comprises a selectivity-determining moiety.

A linker group, e.g., between a topoisomerase inhibitor and the CDP, may comprise a self-cyclizing moiety and a selectivity-determining moiety.

The topoisomerase inhibitor or targeting ligand may be covalently bonded to the linker group via a biohydrolyzable bond (e.g., an ester, amide, carbonate, carbamate, or a phosphate).

The CDP comprises cyclodextrin moieties that may alternate with linker moieties in the polymer chain.

The linker moieties may be attached to topoisomerase inhibitors or prodrugs thereof that are cleaved under biological conditions.

At least one linker that connects the topoisomerase inhibitor or prodrug thereof to the polymer may comprise a group represented by the formula

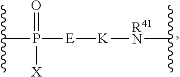

wherein

P is phosphorus;

O is oxygen;

E represents oxygen or NR⁴⁰;

K represents alkenyl;

X is selected from OR⁴² or NR⁴³R⁴⁴; and

R⁴⁰, R⁴¹, R⁴², R⁴³, and R⁴⁴ independently represent hydrogen or optionally substituted alkyl.

E may be NR⁴⁰ and R⁴⁰ is hydrogen.

K may be lower alkylene (e.g., ethylene).

At least one linker may comprise a group selected

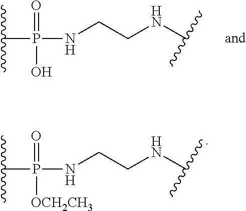 [SMILES: *NCCNP(*)(=O)O ; *NCCNP(*)(=O)OCC]

X may be OR⁴².

The linker group may comprise an amino acid or peptide, or derivative thereof (e.g., a glycine or cysteine, preferably a glycine).

The linker may be connected to the topoisomerase inhibitor through a hydroxyl group. The linker may be connected to the topoisomerase inhibitor through an amino group.

The linker group that connects to the topoisomerase inhibitor may comprise a self-cyclizing moiety, or a selectivity-determining moiety, or both. The selectivity-determining moiety may be a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

Any of the linker groups may comprise a self-cyclizing moiety or a selectivity-determining moiety, or both. The selectivity-determining moiety may be bonded to the self-cyclizing moiety between the self-cyclizing moiety and the polymer.

Any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. The linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

Any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

The present application contemplates a CDP, wherein a plurality of topoisomerase inhibitors are covalently attached to the polymer through attachments that are cleaved under biological conditions to release the therapeutic agents as discussed above, wherein administration of the polymer to a subject results in release of the therapeutic agent over a period of at least 2, 3, 5, 6, 8, 10, 15, 20, 24, 36, 48 or even 72 hours.

The conjugation of the topoisomerase inhibitor to the CDP improves the aqueous solubility of the topoisomerase inhibitor and hence the bioavailability. Accordingly, the topoisomerase inhibitor may have a log P>0.4, >0.6, >0.8, >1, >2, >3, >4, or even >5.

The CDP-topoisomerase inhibitor conjugate of the present application preferably has a molecular weight in the range of 10,000 to 500,000; 30,000 to 200,000; or even 70,000 to 150,000 amu.

The present application contemplates attenuating the rate of release of the topoisomerase inhibitor by introducing various tether and/or linking groups between the therapeutic agent and the polymer. Thus, the CDP-topoisomerase inhibitor conjugates of the present application may be compositions for controlled delivery of the topoisomerase inhibitor.

The CDP and/or CDP-topoisomerase inhibitor conjugate, particle or composition as described herein may have polydispersities less than about 3, or even less than about 2.

The present application may provide an improved delivery of certain topoisomerase inhibitor by covalently attaching one or more topoisomerase inhibitors to a CDP. Such conjugation can improve the aqueous solubility and hence the bioavailability of the topoisomerase inhibitor.

The CDP-topoisomerase inhibitor conjugates, particles and compositions described herein preferably have molecular weights in the range of 10,000 to 500,000; 30,000 to 200,000; or even 70,000 to 150,000 amu. The compound may have a number average (Mₙ) molecular weight between 1,000 to 500,000 amu, or between 5,000 to 200,000 amu, or between 10,000 to 100,000 amu.

The CDP-topoisomerase inhibitor conjugate, particle or composition may be biodegradable or bioerodable.

The topoisomerase inhibitor, e.g., the camptothecin, camptothecin derivative, or prodrug thereof makes up at least 3% (e.g., at least about 5%) by weight of the polymer. The topoisomerase inhibitor, e.g., the camptothecin, camptothecin derivative or prodrug thereof may make up at least 20% by weight of the compound. The topoisomerase inhibitor, e.g., the camptothecin, camptothecin derivative or prodrug thereof may make up at least 5%, 10%, 15%, or at least 20% by weight of the compound.

CDP-topoisomerase inhibitor conjugates, particles and compositions of the present application may be useful to improve solubility and/or stability of the topoisomerase inhibitor, reduce drug-drug interactions, reduce interactions with blood elements including plasma proteins, reduce or eliminate immunogenicity, protect the topoisomerase inhibitor from metabolism, modulate drug-release kinetics, improve circulation time, improve topoisomerase inhibitor half-life (e.g., in the serum, or in selected tissues, such as tumors), attenuate toxicity, improve efficacy, normalize topoisomerase inhibitor metabolism across subjects of different species, ethnicities, and/or races, and/or provide for targeted delivery into specific cells or tissues.

The CDP-topoisomerase inhibitor conjugate, particle or composition may be a flexible or flowable material. When the CDP used is itself flowable, the CDP composition of the application, even when viscous, need not include a biocompatible solvent to be flowable, although trace or residual amounts of biocompatible solvents may still be present.

While it is possible that the biodegradable polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the application that, in a preferred implementation, no solvent is needed to form a flowable composition. Moreover, the use of solvents is preferably avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the CDP-topoisomerase inhibitor conjugate, particle or composition, it should be non-toxic, otherwise biocompatible, and should be used in relatively small amounts. Solvents that are toxic should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

The CDP-topoisomerase inhibitor conjugates, particles and compositions may be soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

The CDP-topoisomerase inhibitor conjugates, particles and compositions described herein, upon contact with body fluids, may undergo gradual degradation. The life of a biodegradable polymer in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with a topoisomerase inhibitor or other material, release of the topoisomerase inhibitor or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the topoisomerase inhibitor or any other material associated with the polymer.

A variety of factors may affect the desired rate of hydrolysis of CDP-topoisomerase inhibitor conjugates, particles and compositions, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the present application contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as a topoisomerase inhibitor or other material loaded in the CDP-topoisomerase inhibitor conjugates, particles or compositions of the present application involves degradation of any such matrix in a 0.1 M PBS solution (pH 7.4) at 37° C., an assay known in the art. For purposes of the present application, the term “PBS protocol” is used herein to refer to such protocol.

In certain instances, the release rates of different CDP-topoisomerase inhibitor conjugates, particles and compositions of the present application may be compared by subjecting them to such a protocol. In certain instances, it may be necessary to process polymeric systems in the same fashion to allow direct and relatively accurate comparisons of different systems to be made. For example, the present application teaches several different methods of formulating the CDP-topoisomerase inhibitor conjugates, particles and compositions. Such comparisons may indicate that any one CDP-topoisomerase inhibitor conjugate, particle or composition releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the present application and release rate protocols.

When formulated in a certain manner, the release rate for CDP-topoisomerase inhibitor conjugates, particles and compositions of the present application may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. The release rate of any incorporated material may be 10,000 ng/day/mg, or even higher. In certain instances, materials incorporated and characterized by such release rate protocols may include therapeutic agents, fillers, and other substances.

The rate of release of any material from any CDP-topoisomerase inhibitor conjugate, particle or composition of the present application may be presented as the half-life of such material in the matrix.

In addition to the protocols for in vitro determination of release rates, in vivo protocols, whereby in certain instances release rates for polymeric systems may be determined in vivo, are also contemplated by the present application. Other assays useful for determining the release of any material from the polymers of the present system are known in the art.

The CDP-topoisomerase inhibitor conjugates, particles and compositions may be formed in a variety of shapes. For example, CDP-topoisomerase inhibitor conjugates may be presented in the form of microparticles or nanoparticles. Microspheres typically comprise a biodegradable polymer matrix incorporating a drug. Microspheres can be formed by a wide variety of techniques known to those of skill in the art. Examples of microsphere forming techniques include, but are not limited to, (a) phase separation by emulsification and subsequent organic solvent evaporation (including complex emulsion methods such as oil in water emulsions, water in oil emulsions and water-oil-water emulsions); (b) coacervation-phase separation; (c) melt dispersion; (d) interfacial deposition; (e) in situ polymerization; (f) spray drying and spray congealing; (g) air suspension coating; and (h) pan and spray coating.

These methods, as well as properties and characteristics of microspheres are disclosed in, for example, U.S. Pat. Nos. 4,438,253; 4,652,441; 5,100,669; 5,330,768; 4,526,938; 5,889,110; 6,034,175; and European Patent 0258780, the entire disclosures of which are incorporated by reference herein in their entireties.

To prepare microspheres, several methods can be employed depending upon the desired application of the delivery vehicles. Suitable methods include, but are not limited to, spray drying, freeze drying, air drying, vacuum drying, fluidized-bed drying, milling, co-precipitation and critical fluid extraction. In the case of spray drying, freeze drying, air drying, vacuum drying, fluidized-bed drying and critical fluid extraction; the components (stabilizing polyol, bioactive material, buffers, etc.) are first dissolved or suspended in aqueous conditions. In the case of milling, the components are mixed in the dried form and milled by any method known in the art. In the case of co-precipitation, the components are mixed in organic conditions and processed as described below. Spray drying can be used to load the stabilizing polyol with the bioactive material. The components are mixed under aqueous conditions and dried using precision nozzles to produce extremely uniform droplets in a drying chamber. Suitable spray drying machines include, but are not limited to, Buchi, NIRO, APV and Lab-plant spray driers used according to the manufacturer's instructions.

The shape of microparticles and nanoparticles may be determined by scanning electron microscopy. Spherically shaped nanoparticles are used in certain applications, for circulation through the bloodstream. If desired, the particles may be fabricated using known techniques into other shapes that are more useful for a specific application.

In addition to intracellular delivery of a topoisomerase inhibitor, it also possible that particles of the CDP-topoisomerase inhibitor conjugates, such as microparticles or nanoparticles, may undergo endocytosis, thereby obtaining access to the cell. The frequency of such an endocytosis process will likely depend on the size of any particle.

The surface charge of the molecule may be neutral, or slightly negative. The zeta potential of the particle surface may be from about −80 mV to about 50 mV.

Generally, the CDP-topoisomerase inhibitor conjugates, particles and compositions described herein can be prepared in one of two ways: monomers bearing topoisomerase inhibitors, targeting ligands, and/or cyclodextrin moieties can be polymerized, or polymer backbones can be derivatized with topoisomerase inhibitors, targeting ligands, and/or cyclodextrin moieties. Exemplary methods of making CDPs and CDP-topoisomerase inhibitor conjugates, particles and compositions are described, for example, in U.S. Pat. No. 7,270,808, the contents of which is incorporated herein by reference in its entirety.

The CDPs described herein can be made using a variety of methods including those described herein. A CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four cyclodextrin moieties and at least four comonomers.

The at least four cyclodextrin moieties and at least four comonomers alternate in the water soluble linear polymer. The method may include providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

The cyclodextrin monomers may comprise linkers to which the topoisomerase inhibitor may be further linked.

The comonomer precursor may be a compound containing at least two functional groups through which reaction and thus linkage of the cyclodextrin moieties is achieved. The functional groups, which may be the same or different, terminal or internal, of each comonomer precursor may comprise an amino, acid, imidazole, hydroxyl, thio, acyl halide, —HC═CH—, —C≡C— group, or derivative thereof. The two functional groups may be the same and are located at termini of the comonomer precursor. A comonomer may contain one or more pendant groups with at least one functional group through which reaction and thus linkage of a therapeutic agent can be achieved. The functional groups, which may be the same or different, terminal or internal, of each comonomer pendant group may comprise an amino, acid, imidazole, hydroxyl, thiol, acyl halide, ethylene, ethyne group, or derivative thereof. The pendant group may be a substituted or unsubstituted branched, cyclic or straight chain C₁-C₁₀ alkyl, or arylalkyl optionally containing one or more heteroatoms within the chain or ring.

The cyclodextrin moiety may comprise an alpha, beta, or gamma cyclodextrin moiety.

The CDP may be suitable for the attachment of sufficient topoisomerase inhibitor such that up to at least 3%, 5%, 10%, 11%, 12%, 13%, 14%, 15%, 20%, 25%, 30%, or even 35% by weight of the CDP, when conjugated, is topoisomerase inhibitor.

The CDP may have a molecular weight of 10,000-500,000 amu. The cyclodextrin moieties may make up at least about 2%, 5%, 10%, 20%, 30%, 50% or 80% of the CDP by weight.

A CDP of the following formula can be made by the scheme below:

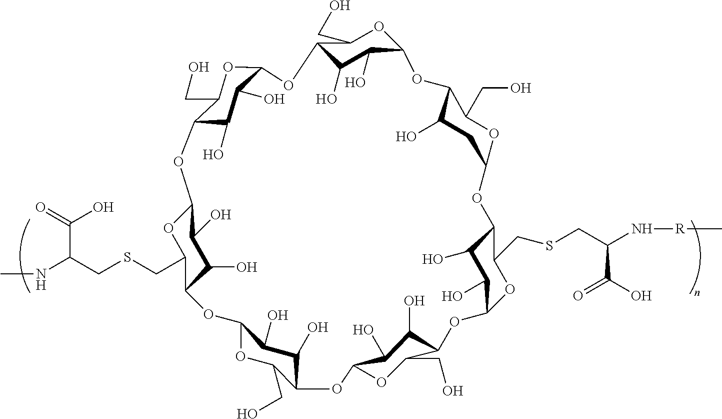

wherein R is of the form:

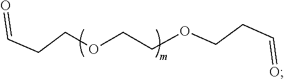

comprising the steps of:
reacting a compound of the formula below:

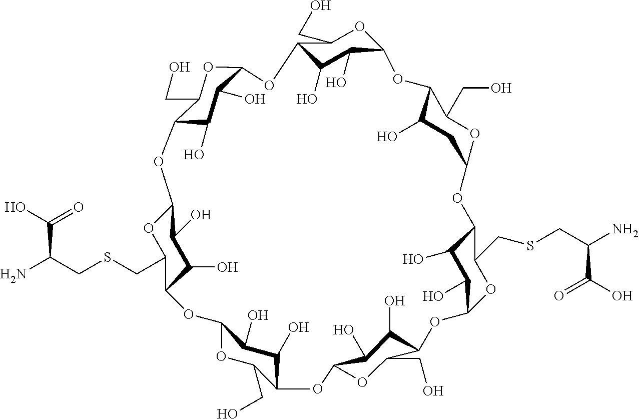 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@@H](C[C@H]8O)O[C@H]1[C@H](O)C2O)C(O)[C@H]7O)C(O)[C@H]6O)C(O)[C@H]5O)C(O)[C@H]4O)C(O)[C@H]3O)C(=O)O]

with a compound of the formula below:

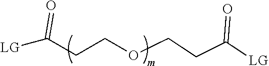

wherein LG represents a leaving group, such as

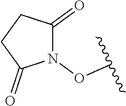 [SMILES: *ON1C(=O)CCC1=O]

(other leaving groups are well known to the skilled person);

wherein the group

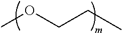

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77) and n is at least four,

in the presence of a non-nucleophilic organic base in a solvent.

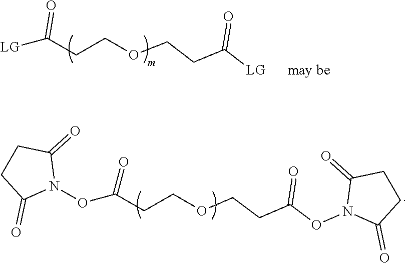

The solvent may be a polar aprotic solvent. The solvent may be DMSO.

The method may also include the steps of dialysis; and lyophylization.

A CDP provided below can be made by the following scheme:

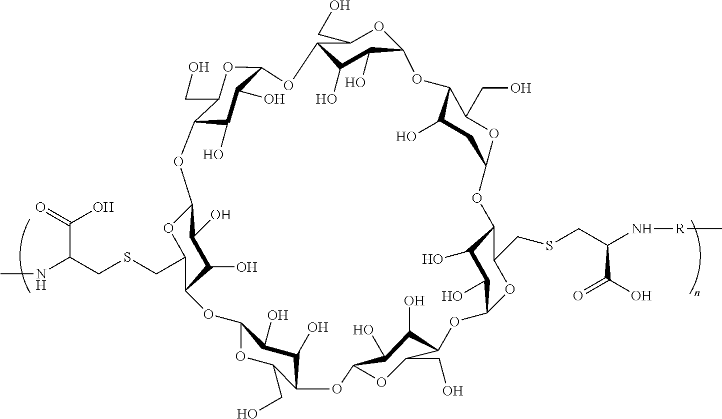

wherein R is of the form:

with a compound provided below:

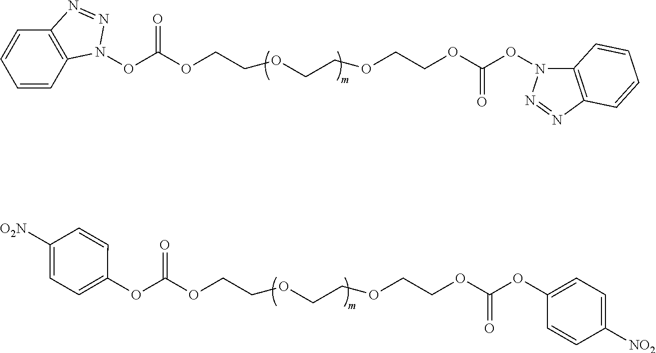

wherein the group

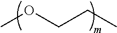

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77);

in the presence of a non-nucleophilic organic base in DMSO;

and dialyzing and lyophilizing the following polymer

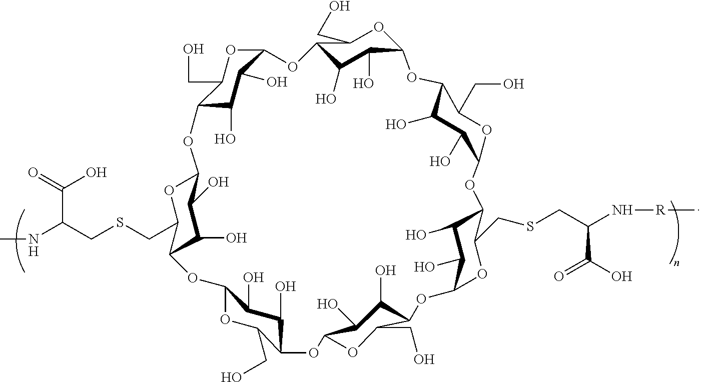

The present application further contemplates CDPs and CDP-conjugates synthesized using CD-biscysteine monomer and a di-NHS ester such as PEG-DiSPA or PEG-BTC as shown in Scheme I.

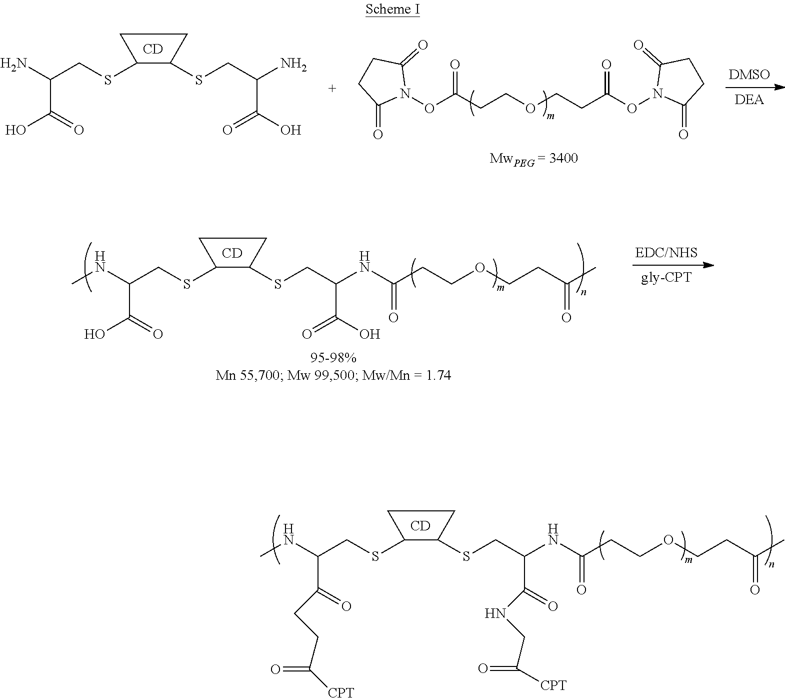

Scheme XIII, as provided above, includes instances where gly-CPT is absent in one or more positions as provided above. This can be achieved, for example, when less than 100% yield is achieved when coupling the CPT to the polymer and/or when less than an equivalent amount of CPT is used in the reaction. Accordingly, the loading of the topoisomerase inhibitor such as camptothecin, by weight of the polymer, can vary. Therefore, while Scheme XIII depicts CPT at each cysteine residue of each polymer subunit, the CDP-CPT conjugate can have less than 2 CPT molecules attached to any given polymer subunit of the COP. For example, the COP-CPT conjugate may include several polymer subunits and each of the polymer subunits can independently include two, one or no CPT attached at each cysteine residue of the polymer subunit. In addition, the particles and compositions can include COP-CPT conjugates having two, one or no CPT attached at each cysteine residue of each polymer subunit of the COP-CPT conjugate and the conjugates include a mixture of COP-CPT conjugates that can vary as to the number of CPTs attached to the gly at each of the polymer subunits of the conjugates in the particle or composition.

A COP-topoisomerase inhibitor conjugate can be made by providing a COP comprising cyclodextrin moieties and comonomers which do not contain cyclodextrin moieties (comonomers), wherein the cyclodextrin moieties and comonomers alternate in the CDP and wherein the CDP comprises at least four cyclodextrin moieties and at least four comonomers; and attaching a topoisomerase inhibitor to the CDP.

One or more of the topoisomerase inhibitor moieties in the CDP-topoisomerase inhibitor conjugate can be replaced with another therapeutic agent, e.g., another anticancer agent or anti-inflammatory agent.

The topoisomerase inhibitor may be attached to the water soluble linear polymer via a linker. The topoisomerase inhibitor may be attached to the water soluble linear polymer through an attachment that is cleaved under biological conditions to release the topoisomerase inhibitor. The topoisomerase inhibitor may be attached to the water soluble linear polymer at a cyclodextrin moiety or a comonomer. The topoisomerase inhibitor may be attached to the water soluble linear polymer via an optional linker to a cyclodextrin moiety or a comonomer.

The cyclodextrin moieties may comprise linkers to which therapeutic agents are linked.

The CDP may be made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. The CDP may be conjugated with a topoisomerase inhibitor such as camptothecin to provide a CDP-topoisomerase inhibitor conjugate.

The method may include providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

The topoisomerase inhibitor may be attached to the CDP via a linker. The linker may be cleaved under biological conditions.

The topoisomerase inhibitor may make up at least 5%, 10%, 11%, 12%, 13%, 14%, 15%, 20%, 25%, 30%, or even 35% by weight of the CDP-topoisomerase inhibitor conjugate.

The comonomer may comprises polyethylene glycol of molecular weight from about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da)), the cyclodextrin moiety comprises beta-cyclodextrin, the theoretical maximum loading of camptothecin on a CDP-camptothecin conjugate is 13%, and camptothecin is 6-10% by weight of the CDP-camptothecin conjugate.

The comonomer precursor may be a compound containing at least two functional groups through which reaction and thus linkage of the cyclodextrin moieties is achieved. The functional groups, which may be the same or different, terminal or internal, of each comonomer precursor may comprise an amino, acid, imidazole, hydroxyl, thio, acyl halide, —HC═CH—, —C≡C— group, or derivative thereof. The two functional groups may be the same and are located at termini of the comonomer precursor. A comonomer may contain one or more pendant groups with at least one functional group through which reaction and thus linkage of a therapeutic agent is achieved. The functional groups, which may be the same or different, terminal or internal, of each comonomer pendant group may comprise an amino, acid, imidazole, hydroxyl, thiol, acyl halide, ethylene, ethyne group, or derivative thereof. The pendant group may be a substituted or unsubstituted branched, cyclic or straight chain C1-C10 alkyl, or arylalkyl optionally containing one or more heteroatoms within the chain or ring.

The cyclodextrin moiety may comprise an alpha, beta, or gamma cyclodextrin moiety.

The topoisomerase inhibitor may be poorly soluble in water.

Administration of the CDP-topoisomerase inhibitor conjugate, particle or composition to a subject may result in release of the topoisomerase inhibitor over a period of at least 6 hours. Administration of the CDP-topoisomerase inhibitor conjugate, particle or composition to a subject may result in release of the topoisomerase inhibitor over a period of 6 hours to a month. Administration of the CDP-topoisomerase inhibitor conjugate, particle or composition to a subject the rate of topoisomerase inhibitor release may be dependent primarily upon the rate of hydrolysis as opposed to enzymatic cleavage.

The CDP-topoisomerase inhibitor conjugate, particle or composition may have a molecular weight of 10,000-500,000 amu.

The cyclodextrin moieties may make up at least about 2%, 5%, 10%, 20%, 30%, 50% or 80% of the polymer by weight.

A CDP-polymer conjugate of the following formula can be made as follows:

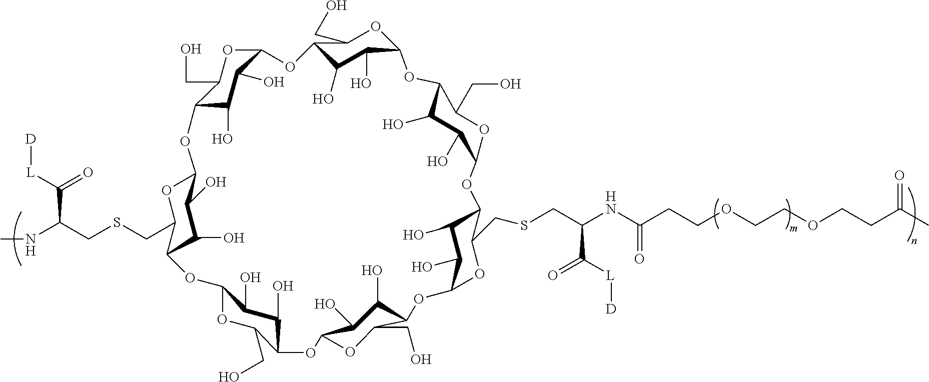

providing a polymer below:

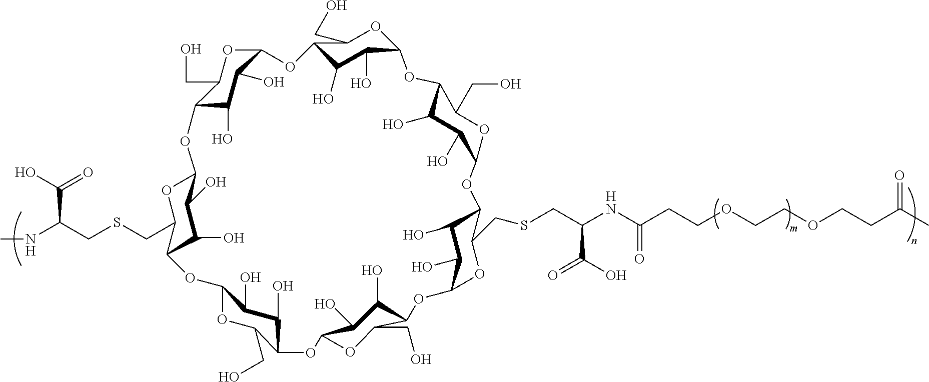

and coupling the polymer with a plurality of L-D moieties, wherein L is a linker, or absent and D is topoisomerase inhibitor such as camptothecin or a camptothecin derivative, to provide:

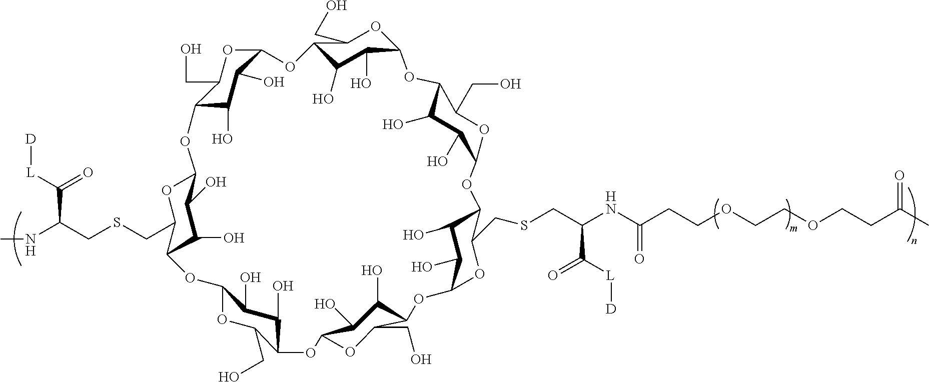

wherein the group

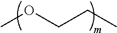

has a Mw of about 2 to about 5 kDa (e.g., from about 2 to about 4.5 kDa, from about 3 to about 4 kDa, or less than about 4 kDa, (e.g., about 3.4 kDa±10%, e.g., about 3060 Da to about 3740 Da), e.g. m may be between about 40 and 100, optionally between about 60 and 90, optionally between about 70 and 80, preferably about 75 and 80, such as about 77) and n is at least 4, wherein on the final product, L can be a linker, a bond, or OH, and D can be a topoisomerase inhibitor (e.g., camptothecin or a camptothecin derivative) or absent.

One or more of the topoisomerase inhibitor moieties in the CDP-topoisomerase inhibitor conjugate can be replaced with another therapeutic agent, e.g., another anticancer agent or anti-inflammatory agent.

The reaction scheme as provided above includes instances where L-D is absent in one or more positions as provided above. This can be achieved, for example, when less than 100% yield is achieved when coupling the topoisomerase inhibitor-linker to the polymer and/or when less than an equivalent amount of topoisomerase inhibitor-linker is used in the reaction.

Accordingly, the loading of the topoisomerase inhibitor, by weight of the polymer, can vary, for example, the loading of the topoisomerase inhibitor can be at least about 3% by weight, e.g., at least about 5%, at least about 8%, at least about 10%, at least about 11%, at least about 12%, at least about 13%, at least about 14%, at least about 15%, or at least about 20%.

At least a portion of the L moieties of L-D may be absent. Each L may be independently an amino acid or derivative thereof (e.g., glycine).

The coupling of the polymer with the plurality of L-D moieties may result in the formation of a plurality of amide bonds.

The CDPs may be random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

Additional Therapeutic Agents

The cyclodextrin-containing polymer-topoisomerase inhibitor conjugate may be used in combination with other known therapies. For example, the cyclodextrin-containing polymer-topoisomerase inhibitor conjugate may be used in combination with one or more anti-cancer agents; hormone and/or steroids; anti-microbials; agents or procedures to mitigate potential side effects from the agent compositions such as cystitis, diarrhea, nausea and vomiting; anti-hypersensitivity agents; an agent that increases urinary excretion and/or neutralizes one or more urinary metabolite; antidiarrheal agents; antiemetic agents; immunosuppressive agents; antihistamines; anti-inflammatories; antipyretics such as those described below.

Exemplary classes of anti-cancer agents include alkylating agents, anti-EGFR antibodies, anti-HER-2 antibodies, small molecules and antibody-drug conjugates, antimetabolites, vinca alkaloids, platinum-based agents, anthracyclines, topoisomerase inhibitors, taxanes, epothilones, antibiotics, immunomodulators, immune cell antibodies, interferons, interleukins, HSP90 inhibitors, angiogenesis inhibitors, anti-androgens, antiestrogens, anti-hypercalcaemia, agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, cRAF kinase inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3, Heat Shock Protein 90, histone deacetylase (HDAC) inhibitors, I-kappa B-alphan kinase inhibitors, imidazotetrazinones, Insulin like growth factor pathway inhibitors, Insulin tyrosine kinase inhibitors, c-Jun-N-terminal kinase, Mitogen-activated protein kinase, MDM2 inhibitors, MEK inhibitors, MMP inhibitors, mTOR inhibitors, Nectin-4 Antibody Drug Conjugates, NGFR tyrosine kinase inhibitors, p38 MAP kinase inhibitors, p56 tyrosine kinase inhibitors, PDGF pathway inhibitors, Phosphatidylinositol 3-kinase inhibitors, Phosphatase inhibitors, PKC inhibitors, PKC delta kinase inhibitors, Proteasome inhibitors, Protein phosphatase inhibitors, Protein tyrosine kinase inhibitors, PTP1B inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, Serine/threonine protein kinase inhibitors, Sterol biosynthesis inhibitors, TROP2 Antibody Drug Conjugates such as the following: alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®) and panitumumab (Vectibix®).

anti-HER-2 antibodies (e.g., trastuzumab (Herceptin®), pertuzumab (Perjeta®)), anti-HER2 small molecules (e.g., tucatinib (Tukysa®), neratinib (Nerlynx®), lapatinib (Tykberb®) and anti-HER2 Antibody Drug Conjugates (e.g. ado-trastuzumab emtansine (Kadcyla®), fam-trastuzumab deruxtecan-nxki (Enhertu®)).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin.

taxanes: paclitaxel (Taxol®), docetaxel (Taxotere®), larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®)).

interleukins: IL-1, IL-2 (Proleukin®), IL-24, IL-6 (Sigosix®), IL-12.

HSP90 inhibitors (e.g., geldanamycin or any of its derivatives). The HSP90 inhibitor may be selected from geldanamycin, 17-alkylamino-17-desmethoxygeldanamycin (“17-AAG”) or 17-(2-dimethylaminoethyl)amino-17-desmethoxygeldanamycin (“17-DMAG”).

angiogenesis inhibitors which include, without limitation A6 (Angstrom Pharmacueticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP O2) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharaceuticals), Combretastatin (Oxigene), CP-751,871 (Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089 (Foretinib) (GlaxoSmithKline), GW786034 (Pazopanib) (GlaxoSmithKline), GT-111 (Vascular Biogenics Ltd.), IMC—1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (Medlmmune), MLN518 (Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TK1258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid, ibrutinib and acalabrutnib.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, Osimertinib (Tagrisso®), erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

Heat Shock Protein 90 (Hsp90) chaperone modulators which include without limitation AUY922, STA-9090, AT113387, MCP-3100, IPI-504, IPI-493, SNX-5422, Debio0932, HSP990, DS-2248, PU-H71, 17-DMAG (Alvespimycin), and XL888.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alphan kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

Insulin like growth factor pathway inhibitors such as IGF inhibitors or IGF receptor (IGFR1 or IGFR2) inhibitors include without limitation, small molecule inhibitors, e.g., OSI-906; anti-IGF antibodies or anti-IGFR antibodies, e.g., AVE-1642, MK-0646, IMC-A12 (cixutumab), R1507, CP-751,871 (Figitumumab).

Insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

Mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl), and trametinib (Mekinist™).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTOR inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

Nectin-4 Antibody Drug Conjugates (enfortumab vedotin-ejfv (PADCEV®).

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

Phosphatidylinositol 3-kinase inhibitors which include without limitation wortmannin, and quercetin dihydrate.

Phosphatase inhibitors which include without limitation cantharidic acid, cantharidin, and L-leucinamide.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC deltan kinase inhibitors which include without limitation rottlerin. polyamine synthesis inhibitors which include without limitation DMFO.

Proteasome inhibitors which include, without limitation aclacinomycin A, gliotoxin and bortezomib (Velcade®).

Protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

Protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives of formula I as generically and specifically described in PCT Publication No.: WO 03/013541 and U.S. Publication No.: 2008/0139587:

Publication No.: 2008/0139587 discloses the various substituents, e.g., R1, R2, etc.

PTP1B inhibitors which include without limitation L-leucinamide.

SRC family tyrosine kinase inhibitors which include without limitation PP1 and PP2.

Syk tyrosine kinase inhibitors which include without limitation piceatannol.

TROP2 Antibody Drug Conjugates (Sacituzumab govitecan (Trodelvy®).

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

Serine/threonine protein kinase inhibitors which include without limitation 2-aminopurine.

Sterol biosynthesis inhibitors which include without limitation squalene epoxidase and CYP2D6.

VEGF pathway inhibitors which include without limitation anti-VEGF antibodies, e.g., bevacizumab (Avastin®), and small molecules, e.g., sunitinib (Sutent®), sorafinib (Nexavar®), ZD6474 (also known as vandetanib) (Zactima™), SU6668, CP-547632, AV-951 (tivozanib) and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

In some cases, the cyclodextrin-containing polymer-topoisomerase inhibitor conjugate can be used in combination with a hormone and/or steriod. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In some cases, the cyclodextrin-containing polymer-topoisomerase inhibitor conjugate can be used in combination with an anti-microbial (e.g., leptomycin B).

In some cases, the cyclodextrin-containing polymer-topoisomerase inhibitor conjugate can be used in combination with an agent or procedure to mitigate potential side effects from the agent compositions such as cystitis, hypersensitivity, diarrhea, nausea and vomiting.

In some cases, the cyclodextrin-containing polymer-topoisomerase inhibitor conjugate can be used in combination with an anti-hypersensitivity agent. Examples of anti-hypersensitivity agents include: corticosteroids, antihistamines and H₂ antagonist; such as dexamethasone diphenhydramine and ranitidine.

Cystitis can be mitigated with an agent that increases urinary excretion and/or neutralizes one or more urinary metabolite. For example, cystitis can be mitigated or treated with MESNA.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, Ianreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present application are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some cases, the cyclodextrin-containing polymer-topoisomerase inhibitor conjugate can be used in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some cases, the cyclodextrin-containing polymer-topoisomerase inhibitor conjugate can be used in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

In some cases, the cyclodextrin-containing polymer-topoisomerase inhibitor conjugate can be used in combination with an antihistamine, such as an H1 or H2 antihistamine, e.g. Acrivastine, Azelastine, Benadryl, Bilastine, Bromodiphenhydramine, Brompheniramine, Buclizine, Carbinoxamine, Cetirizine (Zyrtec), Chlorodiphenhydramine, Chlorpheniramine, Clemastine, Cyclizine, Cyproheptadine, Desloratadine, Dexbrompheniramine, Dexchlorpheniramine, Dimenhydrinate (most commonly used as an antiemetic), Dimetindene, Diphenhydramine, Doxylamine, Ebastine, Embramine, Fexofenadine (Allegra/Telfast), Hydroxyzine (Vistaril), Levocabastine (Livostin/Livocab), Levocetirizine (Xyzal), Loratadine (Claritin), Meclizine, Mirtazapine, Olopatadine, Orphenadrine, Phenindamine, Pheniramine, Phenyltoloxamine, Promethazine, Pyrilamine, Quetiapine, Rupatadine (Alergoliber), Trazodone, Tripelennamine, Triprolidine; or Cimetidine, Famotidine, Lafutidine, Nizatidine, Ranitidine, Roxatidine, Tiotidine.

In some cases, the cyclodextrin-containing polymer-topoisomerase inhibitor conjugate can be used in combination with an anti-inflammatory, such as salicylates (e.g. aspirin (acetylsalicylic acid), diflunisal (Dolobid), salicylic acid and its salts, Salsalate (Disalcid)); propionic acid derivatives (e.g. ibuprofen, Dexibuprofen, Naproxen, Fenoprofen, Ketoprofen, Dexketoprofen, Flurbiprofen, Oxaprozin, Loxoprofen); acetic acid derivatives (e.g. Indomethacin, Tolmetin, Sulindac, Etodolac, Ketorolac, Diclofenac, Aceclofenac, Bromfenac, Nabumetone); Enolic acid (oxicam) derivatives (e.g. Piroxicam, Meloxicam, Tenoxicam, Droxicam, Lornoxicam, Phenylbutazone (Bute)); anthranilic acid derivatives (fenamates) (e.g. Mefenamic acid, Meclofenamic acid, Flufenamic acid, Tolfenamic acid); or Clonixin, Licofelone, H-harpagide in figwort or devil's claw.

In some cases, the cyclodextrin-containing polymer-topoisomerase inhibitor conjugate can be used in combination with an antipyretic, such as NSAIDs (e.g. ibuprofen, naproxen, ketoprofen, and nimesulide); aspirin and related salicylates such as choline salicylate, magnesium salicylate, and sodium salicylate; paracetamol (acetaminophen), nabumetone, phenazone (antipyrine).

When employing the use, methods or compositions, other agents used in the modulation of tumor growth or metastasis in a clinical setting, such as antiemetics, can also be administered as desired.

### BRIEF DESCRIPTION OF THE FIGURES

FIG. 1: ANTITUMOUR ACTIVITY OF CRLX-101 COMPARED TO IRINOTECAN IN VARIOUS SUBCUTANEOUS HUMAN XENOGRAFT MODELS IN ATHYMIC MICE

FIG. 2: ANTITUMOUR ACTIVITY OF CRLX-101 IN 2 OVARIAN HUMAN XENOGRAFT MODELS IN ATHYMIC MICE

FIG. 3: CRLX-101 IS SYNERGISTIC WITH PACLITAXEL IN THE SKOV-3 HUMAN OVARIAN XENOGRAFT MODEL

FIG. 4: CRLX-101 IMPROVES CHEMORADIOTHERAPY IN THE HT-29 COLORECTAL XENOGRAFT MODEL

FIG. 5: CRLX-101 INHIBITS CA9 IN THE HT-29 COLORECTAL XENOGRAFT MODEL

FIG. 6: TUMOUR GROWTH AND TUMOUR-INITIATING CAPACITY OF HUMAN TRIPLE NEGATIVE BREAST CANCER SUM159 ORTHOTOPIC TUMOUR-BEARING MICE ADMINISTERED CRLX-101 IN COMBINATION WITH BEVACIZUMAB

FIG. 7: TIME COURSE OF DNA DAMAGE IN RAT BONE MARROW AND MOUSE TUMOUR

FIG. 8: EFFECTS OF OLAPARIB DOSE DELAY ON PERIPHERAL BLOOD CELL COUNTS IN RATS

FIG. 9: EFFECTS OF CRLX-101 PLUS OLAPARIB IN A SMALL-CELL LUNG CANCER XENOGRAFT MODEL

FIG. 10: TIME COURSE OF HIF INHIBITION IN THE HCT-116 HUMAN COLORECTAL XENOGRAFT MODEL

FIG. 11: ANTITUMOUR ACTIVITY OF CRLX-101 COMBINED WITH BEVACIZUMAB, AFLIBERCEPT OR PAZOPANIB IN THE HUMAN A2780 OVARIAN TUMOUR MODEL IN ATHYMIC MICE

FIG. 12: SURVIVAL OF HUMAN OVARIAN A2780 TUMOUR-BEARING MICE ADMINISTERED CRLX-101 IN COMBINATION WITH BEVACIZUMAB, AFLIBERCEPT OR PAZOPANIB

FIG. 13: TUMOUR GROWTH AND SURVIVAL OF ORTHOTOPIC HUMAN OVARIAN SKOV-3-13 TUMOUR-BEARING MICE ADMINISTERED CRLX-101 IN COMBINATION WITH BEVACIZUMAB

FIG. 14: HIF-1A IMMUNOHISTOCHEMISTRY IN ORTHOTOPIC HUMAN OVARIAN SKOV-3-13 TUMOURS ADMINISTERED CRLX-101 IN COMBINATION WITH BEVACIZUMAB

FIG. 15: TUMOUR GROWTH AND SURVIVAL OF HUMAN RENAL CELL CARCINOMA 786-O TUMOUR-BEARING MICE ADMINISTERED CRLX-101 IN COMBINATION WITH BEVACIZUMAB

FIG. 16: HUMAN PLASMA AUC AND CLEARANCE FOR INDIVIDUAL SUBJECTS MEASURED AFTER THE FIRST DOSE AND THE ELEVENTH DOSE

FIG. 17: CPT LOCALISATION AND PHARMACODYNAMIC EFFECTS IN GASTRIC CANCER TUMOURS AFTER FIRST ADMINISTERED DOSE OF CRLX-101

FIG. 18: CRLX-101 LOCALISATION AND PHARMACODYNAMIC EFFECTS IN OVARIAN CANCER TUMOURS AFTER FIRST ADMINISTERED DOSE OF CRLX-101

FIG. 19: CAPAN-1 (schedule 1) % VIABILITY COMPARED TO VEHICLE CONTROLS

FIG. 20: CAPAN-1 (schedule 2) % VIABILITY COMPARED TO VEHICLE CONTROLS

FIG. 21: DU145% VIABILITY COMPARED TO VEHICLE CONTROLS

FIG. 22: HS 766T % VIABILITY COMPARED TO VEHICLE CONTROLS

FIG. 23: OVCAR3% VIABILITY COMPARED TO VEHICLE CONTROLS

FIG. 24: PANC-1% VIABILITY COMPARED TO VEHICLE CONTROLS

FIG. 25: NCI-H510A % VIABILITY COMPARED TO VEHICLE CONTROLS

FIG. 26: NUGC4% VIABILITY COMPARED TO VEHICLE CONTROLS

FIG. 27: OAW28% VIABILITY COMPARED TO VEHICLE CONTROLS

FIG. 28: SKOV3 TUMOR VOLUME OVER TIME

FIG. 29: NCI-N87 TUMOR VOLUME OVER TIME

FIG. 30: PC TUMOR VOLUME OVER TIME

FIG. 31: 22RV1 TUMOR VOLUME OVER TIME

FIG. 32: STUDY DESIGN/SCHEMA (EXAMPLE 25)

### EXAMPLES

So that this present disclosure can be more fully understood, the following examples are set forth. These examples are illustrative only and are not intended to limit the scope of the present disclosure in any way.

CRLX-101 and olaparib were supplied under a Collaborative Research and Development Agreement among National Cancer Institute (NCI), Bluelink (previously provided by Cerulean) and Astra Zeneca.

Nonclinical Studies

The anti-tumour activity of CRLX-101 has been studied in a number of human xenograft models, including ovarian cancer, colon cancer, anthracycline-resistant breast cancer, lymphoma, sarcoma, pancreatic cancer, SCLC, and NSCLC. In every case, CRLX-101 was statistically superior to comparators in either delay of tumour progression or complete response rate.

The MTD of CRLX-101 was found to be dependent on both dose and cycle intensity CRLX-101 was generally well tolerated at human equivalent doses up to 30 mg/m² when administered as 3 weekly injections to rats and dogs. The estimated HED of the severely STD ranged from 42 to 51 mg/m² given as weekly injections.

The primary toxicities observed above the MTD were bone marrow suppression, anorexia, mucositis, and local inflammatory changes, all of which resolve by 14 days following the last dose. In fact, anorexia was reversed within 4 days after the last dose and animal weights recovered to that of their untreated controls. A small degree of pancreatitis and myocarditis was also observed at doses greater than the MTD. The CDP polymer alone was not associated with any overt clinical or histological toxicity.

The pharmacokinetics (PK) of the total and unconjugated CPT demonstrated multi-compartment (2 or 3 compartments) distribution with a rapid plasma equilibration (˜1 hour) and a terminal half-life (T½) for both total and unconjugated CPT of ˜25 hours. Pharmacokinetics at Days 1 and 15 demonstrate no accumulation of either total or unconjugated CPT following 3 weekly doses. Tissue tumour concentrations of total and unconjugated CPT estimated from mouse tissue distribution data at a HED of 30 mg/m² are expected to be above the 50% inhibitory concentrations (IC50) for LS174t colon cancer for up to 120 hours (71%) of the dosing interval.

These data indicate CRLX-101 is active in a variety of human tumour cell lines, well tolerated at a HED of 30 mg/m², and may provide extended inhibitory concentrations of CPT to tumours.

CRLX-101 was evaluated in the presence of human S9 liver microsomal fraction to identify potential metabolites. The results indicated no metabolites were detected at time points up to 120 minutes. CRLX-101 was also evaluated for the ability to activate cytochrome P450 enzymes in human hepatocytes. The results indicated no induction of the following cytochrome P450 enzymes: CYP1A2, CYP2C9, or CYP3A4.

### Example 1: CRLX-101 Monotherapy Studies in Xenograft Models Bearing Human Tumour Implants

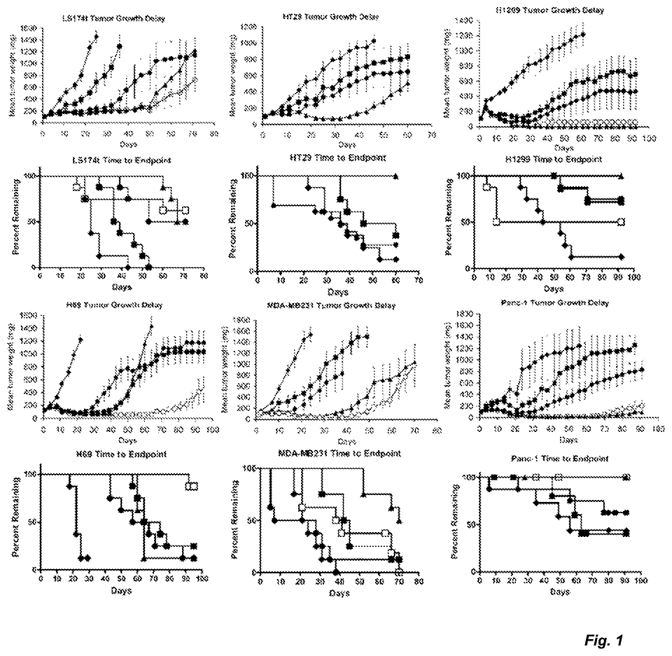

Studies were performed to evaluate the antitumour activity of CRLX-101 in athymic nude mice bearing human tumour implants. The tumour growth plots are illustrated in FIG. 1 (Antitumour Activity of CRLX-101 Compared to Irinotecan in Various Subcutaneous Human Xenograft Models in Athymic Mice) from a number of in vivo studies of CRLX-101 antitumour activity versus irinotecan (CPT-11) in various xenograft tumour types. In every case, CRLX-101 was superior to CPT-11 when dosed weekly for 3 weeks.

Inhibition of 6 different human xenografts by CRLX-101.

Mice were treated with vehicle or the internal positive control irinotecan administered intraperitoneally on Days 0, 7, and 14 at 100 mg/kg. CRLX-101 was administered intravenously as a single high dose or as three weekly doses on Days 0, 7, and 14 at a high or low level. Dose levels were adjusted based on differences in dose limiting toxicity observed in the different models: High dose=25.8 mg/kg (LS174T, H1299, MDA-MB-231), 20.7 mg/kg (HT29, Panc-1), or 16.1 mg/kg (H69). Low dose=16.9 mg/kg (LS174T, H1299, MDA-MB-231), 12.9 mg/kg (HT29, Panc-1), or 9.7 mg/kg (H69). Tumour growth curves are means with bars indicating standard error. Survival is indicated as the percentage of animals remaining on study because mice bearing tumours that reached a predetermined cutoff size were removed from the experiment. Endpoint tumour size was chosen to maximise the number of tumour doublings within the exponential growth phase in the control animals. Endpoint size varied for each cell line and was set at 1500 mm³ for LS174T and MDA-MB-231, 1200 mm3 for H1299, H69, and Panc-1, and 1000 mm³ for HT29. Dosages of CRLX-101 as described herein are expressed in mg of camptothecin, as opposed to mg of conjugate.

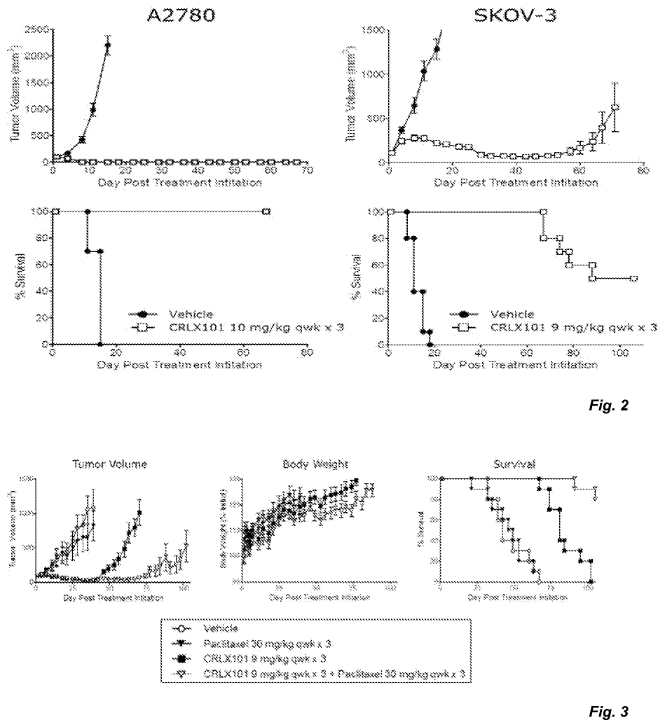

CRLX-101 has also been shown to be active in 2 ovarian human xenograft models: the A2780 model and the SKOV-3 model. Ten nude mice bearing A2780 tumours were dosed with either vehicle (saline) or 10 mg/kg CRLX-101 once weekly for 3 weeks (see FIG. 2; Antitumour Activity of CRLX-101 in 2 Ovarian Human Xenograft Models in Athymic Mice). CRLX-101 treatment resulted in 100% of the mice surviving tumour-free at the end of the study (Day 67). In contrast, all of the mice treated with saline reached the endpoint (tumour volume ≥1000 mm3) by Day 18. Ten nude mice bearing SKOV-3 tumours were dosed with either vehicle (saline) or 9 mg/kg CRLX-101 once weekly for 3 weeks. CRLX-101 treatment resulted in 3 partial responses and 5 survivors at the end of the study (Day 106). The median survival was 87 days after treatment initiation. In contrast, all of the mice treated with saline reached the endpoint by Day 18 and the median survival was 10 days.

Inhibition of 2 different human ovarian xenografts by CRLX-101. Mice were treated intravenously with vehicle or CRLX-101 as three weekly doses on Days 1, 8, and 15. Mice bearing A2780 tumours were treated with 10 mg/kg CRLX-101 and mice bearing SKOV-3 tumours were treated with 9 mg/kg CRLX-101. Tumour growth curves (top) are means with bars indicating standard error. Survival is indicated as the percentage of animals remaining on study because mice bearing tumours that reached a predetermined cutoff size (1000 mm3) were removed from the experiment.

### Example 2: CRLX-101 Combination with Paclitaxel in an Ovarian Xenograft Model Bearing Human Tumour Implants

The potential for combining CRLX-101 with Paclitaxel is shown in FIG. 3 (CRLX-101 with Paclitaxel in the SKOV-3 Human Ovarian Xenograft Model). In this study, CRLX-101 was compared to the MTD of Paclitaxel alone or in combination with a sub-maximal dose of CRLX-101 in the taxane-resistant human ovarian xenograft model SKOV-3. As shown in the Table below, CRLX-101 alone dosed intravenously at a sub-maximal dose of 9 mg/kg once weekly for 3 weeks exhibited significant activity, with a median time to endpoint (TTE) of 75.5 days corresponding to a tumour growth delay (TGD) of 108%.

This regimen yielded 7 partial regressions (PRs), but no animals survived to the end of the study (Day 105). The 30 mg/kg paclitaxel monotherapy dosed once weekly for 3 weeks produced a low median TTE of 42.8 days (TGD of 18%), but yielded 1 tumour-free survivor (TFS). CRLX-101 (9 mg/kg) combined with 30 mg/kg Paclitaxel (both dosed weekly for 3 weeks) produced a median TTE of 105 days, corresponding to TGD of >189%. Relative to the corresponding monotherapies, the combination significantly prolonged survival (log-rank test). The combination generated 8 survivors at the end of the study and 8 PRs. The combination was well tolerated; no deaths or group mean body weight losses (BWL) occurred in this study.

The combination of CRLX-101 and Paclitaxel provides particularly effective results in the SKOV-3 ovarian xenograft model. Tumour growth delay and survival are greater for the combination than either treatment alone or the sum of both individual treatments. All treatments were given weekly for 3 weeks at the doses indicated in the figure legend. Tumour growth curves are means with bars indicating standard deviation. Body weight curves are means relative to the initial body weight, with bars indicating standard deviation. Survival is indicated as the percentage of animals remaining on study because mice bearing tumours that reached a predetermined cut off size (1500 mm3) were removed from the experiment. CRLX-101 dose is in camptothecin equivalent.

TABLE
CRLX-101 is Synergistic with Paclitaxel in the SKOV-3 Human Ovarian Xenograft Model

| | CRLX-101 | | | Paclitaxel | | | | Response Summary | | | | | |
|---|---|---|---|---|---|---|---|---|---|---|---|---|---|
| | Dose | | | Dose | | | | Median | TGD | | | | BWL |
| Gr | (mg/kg) | Schedule | Route | (mg/kg) | Schedule | Route | N | TTE (Days) | (%) | PR | CR | TFS | max (%) |
| 1 | — | — | — | — | — | — | 10 | 36.3 | — | 0 | 0 | 0 | 0 |
| 2 | 9 | QW × 3 | IV | — | — | — | 10 | 75.5 | 108 | 7 | 0 | 0 | 0 |
| 3 | — | — | — | 30 | QW × 3 | IV | 10 | 42.8 | 18 | 0 | 1 | 1 | 0 |
| 4 | 9 | QW × 3 | IV | 30 | QW × 3 | IV | 10 | >105.0 | >189 | 8 | 2 | 0 | 0 |

N = Number of animals in a group
TTE = Time to endpoint (1500 mm3) in days, relative to day of first dose. For animals treated with the combination, TTE was never achieved and was therefore greater than the duration of the study (105 days post initial treatment).
TGD (%) = Percent tumour growth delay vs. control group = [(T − C)/C] × 100. The maximum possible TGD in this study is 189%.
PR = Partial regressions, defined as a tumour volume <50% of initial tumour volume for 2 or more consecutive measurements.
CR = Complete regressions, defined as a tumour volume <13.5 mm3 for 2 or more consecutive measurements.
TFS = Number of animals classified as tumour-free survivors, i.e., CRs at the end of study.
BWLmax (%) = Maximum body weight loss, lowest group mean body weight as % decline from Day 1.

### Example 3: CRLX-101 Combination with Chemoradiotherapy in a Colorectal Xenograft Model Bearing Human Tumour Implants

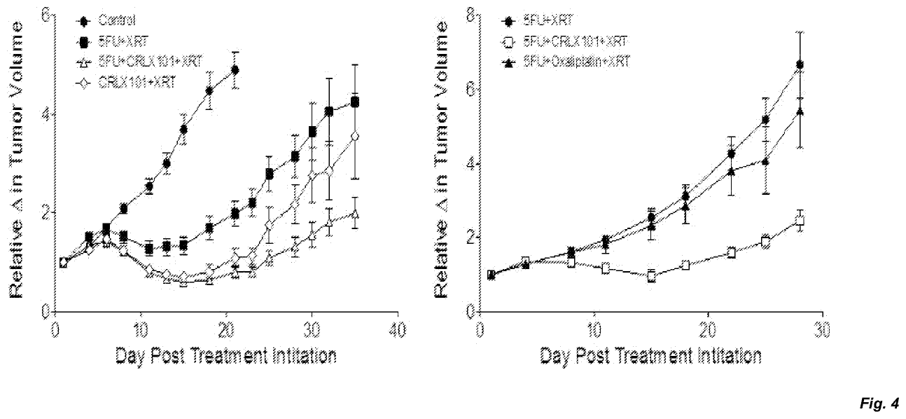

CRLX-101 was also shown to improve tumour growth delay when combined with standard CRT in the HT-29 colorectal xenograft model. As shown in FIG. 4 (CRLX-101 Improves Chemoradiotherapy in the HT-29 Colorectal Xenograft Model; left), CRLX-101-based CRT regimens delayed tumour growth more than corresponding CRT with 5-fluorouracil (5 FU) (CRLX-101+radiation therapy [XRT] vs 5-FU+XRT: p-value=0.0006), and the addition of 5-FU enhanced the anti-proliferative effect of CRT using CRLX-101 (5-FU+CRLX-101+XRT vs CRLX-101+XRT: p value=0.001). Furthermore, as shown in FIG. 4 (right), the tumour growth delay in response to treatment with CRLX-101 plus 5-FU plus XRT was superior to Oxaliplatin plus 5-FU plus XRT.

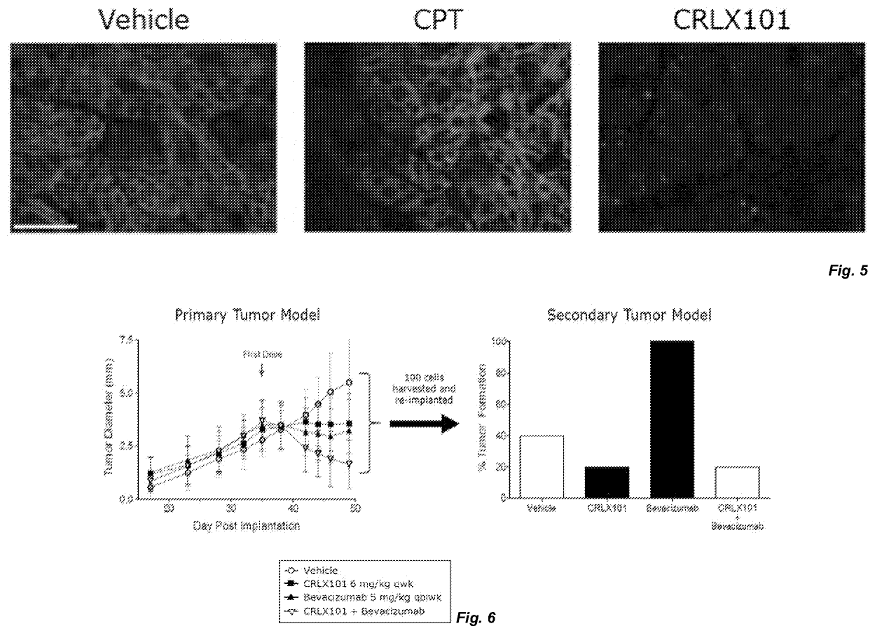

In addition, as shown in FIG. 5 (CRLX-101 Inhibits CA9 in the HT-29 Colorectal Xenograft Model), CRLX-101 but not CPT inhibits the HIF-1a target carbonic anhydrase 9 (CA9) in the HT-29 colorectal xenograft model 7 days after a single IV treatment, consistent with the durable inhibition of HIF-1a by CRLX-101 as described above. Given the role of HIF-1a in resistance to XRT, inhibition of HIF-1a by CRLX-101 may help explain the greater effect of CRLX-101 on tumour growth delay.

Immunodeficient Nu/Nu mice were implanted subcutaneously with human HT-29 colorectal cancer tumour cells and were administered a single dose of CRLX-101 (5 mg/kg), 5-FU (20 mg/kg), radiotherapy (XRT; 3 daily fractions of 5 Gy) or some combination as indicated starting on Day 1 and tumour volumes were measured twice weekly. Tumour growth curves are means with bars indicating standard error of the mean. CRLX-101 dose is in camptothecin equivalent.

Nude mouse xenograft models in which human HT-29 colorectal cancer tumour cells were implanted subcutaneously in mice were administered a single dose of CRLX-101 (5 mg/kg), CPT (0.5 mg/kg) or vehicle. Tumours were harvested 7 days after treatment and immunostained with anti-CA9 antibody. Scale bar=50 μm.

### Example 4: CRLX-101 Inhibits Cancer Stem Cells in Orthotopic Triple-Negative Breast Xenograft Model

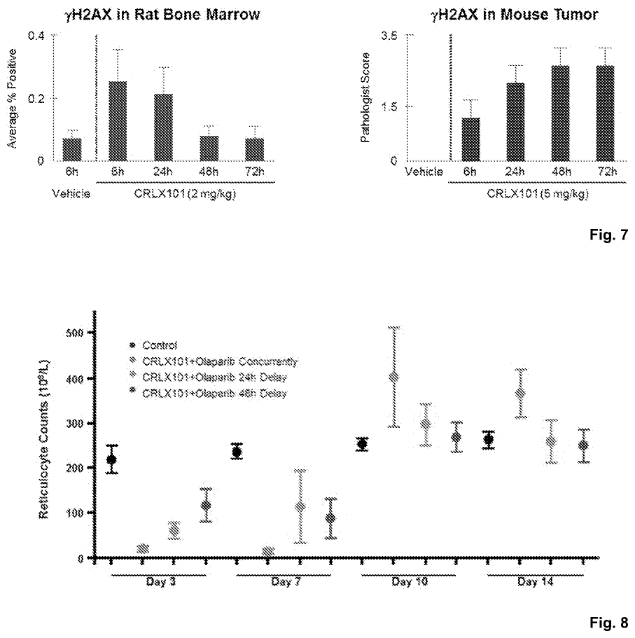

Evidence in the literature suggests a key role of hypoxia and, more specifically, HIF-1α in the stimulation of cancer stem cells (CSCs) in tumours. Such stimulation of CSCs could increase drug resistance and metastasis, and may be further enhanced by treatment with antiangiogenic drugs, which can increase tumour hypoxia by reducing blood flow to tumours. Inhibition of HIF-1α might therefore be beneficial to patients by reducing CSCs in tumours. We therefore investigated the effect of CRLX-101 alone and in combination with the antiangiogenic drug Bevacizumab in the SUM159 triple-negative breast cancer model. Previously published studies have shown that Bevacizumab increases the CSC population mediated primarily by hypoxia induced up-regulation of HIF-1α. Mice bearing SUM159 tumours were dosed for 2 weeks and tumour volume was measured and plotted in FIG. 6 (Tumour Growth and Tumour-Initiating Capacity of Human Triple Negative Breast Cancer SUM159 Orthotopic Tumour-Bearing Mice Administered CRLX-101 in Combination with Bevacizumab; left). These data show at least an additive effect of combining CRLX-101 plus Bevacizumab, and are consistent with observations in the ovarian models, above. At the end of these 2 weeks, tumours were extracted and 100 tumour cells were implanted orthotopically into new, untreated mice. This secondary group of mice was observed for 90 days without treatment, and the percentage of these mice that grew new tumours is plotted in FIG. 7 (right) as the percentage of tumour formation, a functional assay for CSC enrichment. This experiment demonstrates that, as expected, pre treatment with Bevacizumab led to enhanced tumour-initiating capacity compared to control. In contrast, pre-treatment with CRLX-101 led to a reduction in tumour-initiating capacity compared to control and prevented the increase in tumour initiating capacity when combined with Bevacizumab, thus demonstrating that CRLX-101 can reduce the formation of CSCs induced by pre-treatment with Bevacizumab.

Right tumour growth delay of the orthotopic SUM159 triple-negative breast cancer xenograft model is greater for the combination of CRLX-101 plus Bevacizumab than either treatment alone. Mice were dosed for two weeks at the dose level and frequency indicated in the legend. Tumour growth curves represent mean tumour volume and error bars represent standard deviation. Left, secondary group of mice was observed for 90 days without treatment, and the percentage of these mice that grew new tumours is plotted (right) as the percentage of tumour formation, a functional assay for CSC enrichment.

### Example 5: CRLX-101 Combination with Olaparib in Xenograft Models Bearing Human Tumour Implants

Wild type Wistar rats were administered a single IV dose of CRLX-101, and DNA damage was quantified in femur bone marrow sections as the % cells positive for phosphorylated H2A histone family, member X (yH2AX), a marker of DNA double-strand breaks, at various time points after CRLX-101 administration. A dose of 2 mg/kg CRLX-101 was administered, equivalent to 11.8 mg/m². As shown in FIG. 7 (Time Course of DNA Damage in Rat Bone Marrow and Mouse Tumour; left), γH2AX is transiently elevated but returns to baseline 48 hours after CRLX-101 administration.

As shown in FIG. 7 (right), in contrast to what was observed in bone marrow, DNA double strand breaks, as measured by immunohistochemistry of γH2AX, increased over time and remained maximal 72 hours after IV administration of 5 mg/kg CRLX-101 (equivalent to 15 mg/m²) to male nude mice implanted with human NCI-H417a small-cell lung cancer tumour cells. Therefore, it may be possible to retain improved efficacy in the tumour while avoiding combined toxicity in the bone marrow, by dosing CRLX-101 first, and delaying administration of Olaparib by at least two days.

Time course of DNA double strand breaks, as measured by γH2AX immunohistochemistry, following a single dose of CRLX-101 in rat bone marrow (left) and tumours from mice implanted with NCI-H417a SCLC tumour cells (right). Rats were administered a dose of 2 mg/kg, equivalent to 11.8 mg/m², and mice were administered a dose of 5 mg/kg, equivalent to 15 mg/m².

In order to test this hypothesis, wild type Wistar rats were administered a single IV dose of CRLX-101 at 2 mg/kg CRLX-101, equivalent to 11.8 mg/m², and daily oral Olaparib (100 mg/kg, daily for 7 days) either concurrently or following a 24- or 48-hour delay after administration of CRLX-101. Peripheral blood was collected from tail vein and analysis was performed on the Siemens Advia 2120i haematology analyser. As shown in FIG. 8 (Effects of Olaparib Dose Delay on Peripheral Blood Cell Counts in Rat; legend labels from top to bottom correspond to datapoints from left to right within a given day), delaying the Olaparib dose by 24-48 hours is sufficient to provide significant sparing effect on peripheral blood cells. In all cases, peripheral blood counts recovered by Day 10, 3 days following the last dose of oral Olaparib.

Peripheral blood counts in wild type Wistar rats measured following a single dose of 2 mg/kg CRLX-101 (equivalent to 11.8 mg/m²) and 100 mg/kg oral Olaparib daily for 7 days, administered concurrently or following a 24 h or 48 h delay after administration of CRLX-101.

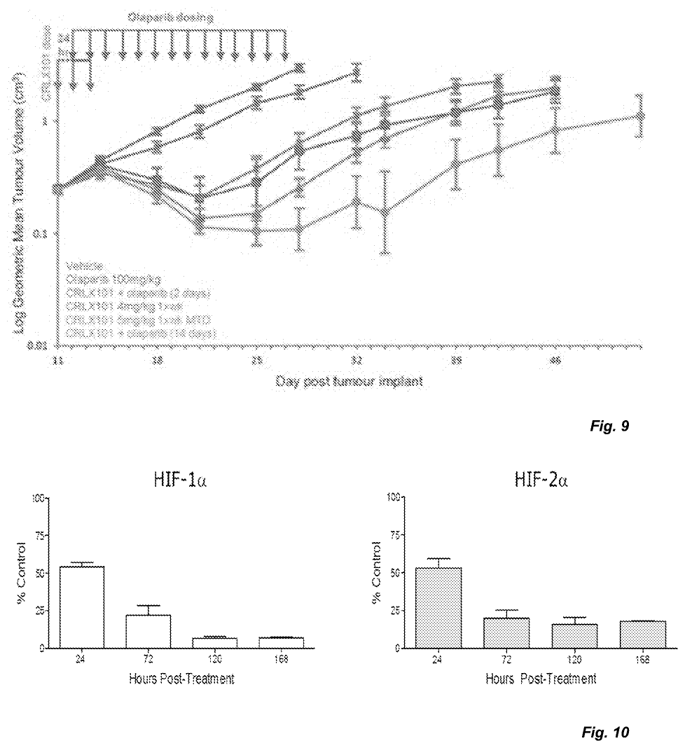

A tumour growth delay study was carried out in nude mice implanted with human NCI-H417a small-cell lung cancer tumour cells (FIG. 9; Effects of CRLX-101 Plus Olaparib in a Small-Cell Lung Cancer Xenograft Model—legend depicts lines from top to bottom as observed at, for example, 26 days post tumour implant). The NCI-H417a model is known to possess wild type (non-mutated) BRCA1 and BRCA2 genes and is therefore relatively non-responsive to Olaparib monotherapy. Mice were administered a single IV dose of either 4 or 5 mg/kg CRLX-101 alone or in combination with either 2 days or 14 days of daily oral Olaparib at 100 mg/kg. In all cases, Olaparib was administered with a 24-hour delay following CRLX-101 administration, to reduce bone marrow suppression. Significantly, 5 mg/kg CRLX-101, equivalent to 15 mg/m², the monotherapy MTD in patients, was tolerated when combined with 14 days of Olaparib with a 24-hour delay, and the combination resulted in superior tumour growth delay. These data suggest that combining CRLX-101 with Olaparib with a delay of 1-2 days should be combinable in the clinic at therapeutically relevant doses of both drugs.

Tumour growth delay following administration of a single IV dose of either 4 or 5 mg/kg CRLX-101 alone or in combination with either 2 days or 14 days of daily oral Olaparib at 100 mg/kg in nude mice implanted with the human NCI-H417a small-cell lung cancer tumour.

### Example 6: CRLX-101 Inhibits HIF and is Synergistic with Antiangiogenic Drugs

Hypoxia-inducible factor 1 alpha and 2 alpha are hypoxia-driven transcription factors that are known to be up-regulated in a large variety of solid tumours.10 High expression of HIF-1α, in particular, is known to correlate with poor prognosis in most tumour types, in part because HIF-1α can turn on a number of cancer cell survival mechanisms including drug resistance, cell survival, angiogenesis, migration, and metastases. Published data have demonstrated that sustained inhibition of topo-1 can lead to inhibition of HIF-1α.

We therefore sought to determine whether CRLX-101, which has been shown preclinically to result in prolonged inhibition of topo-1 in tumours, can inhibit HIF. The human tumour models A2780 ovarian, SKOV-3 ovarian, HCT-116 colon cancer, DU-145 prostate cancer, NCI-H1299 NSCLC, NCI-H520 squamous NSCLC, and Caki-1 were each administered a single dose of CRLX-101 at 6 mg/kg, below the MTD. Tumour HIF-1α protein levels (as measured using western blots) were lower following CRLX-101 treatment in these 7 different tumour types. The Table below shows HIF-1α levels relative to vehicle treated control tumours 72 hours after CRLX-101 administration.

TABLE
HIF-1α Protein Levels 72 hours After a Single IV
Administration of CRLX-101 at a
Dose of 6 mg/kg, mean ± SD (n = 2-3)

| | HIF-1α % of Control, |
|---|---|
| Tumour Type | Mean ± SD (n = 2-3) |
| A2780 ovarian | 23 ± 4 |
| SKOV-3 ovarian | 27 ± 12 |
| HCT-116 colon | 65 ± 6 |
| DU-145 prostate | 55 ± 7 |
| H1299 NSCLC | 27 ± 1 |
| H520 NSCLC | 66 ± 19 |
| Caki-1 renal cell | 56 ± 16 |

The time course of HIF inhibition was studied in the HCT-116 colorectal model after a single administration of CRLX-101. In this study, we measured protein levels of both HIF-1α and HIF 2α following a single administration of 6 mg/kg CRLX-101, using western blot methods.

FIG. 10 (Time Course of HIF Inhibition in the HCT-116 Human Colorectal Xenograft Model) shows that both HIF-1α and HIF-2α were inhibited in this model, with inhibition of >90% for HIF-1α and >80% for HIF-2α, each lasting at least 1 week after a single dose of CRLX-101. Durable inhibition of both HIF-1α and HIF-2α of ≥50% for at least a week following a single dose of CRLX-101 was also observed in the A2780 ovarian xenograft model. In contrast, a single dose of Topotecan was unable to significantly inhibit either HIF-1α or HIF-2α over the same time period.

Nude mouse xenograft models in which human HCT-116 colorectal cancer tumour cells were implanted subcutaneously in mice were administered a single dose of 6 mg/kg CRLX-101 or saline (vehicle) to the control group and tumours were collected at four different time points: 24, 72, 120 and 168 hours after treatment. Tumours were flash-frozen and HIF-1α or HIF-2α protein levels were measured via western blot analysis, quantified using infrared fluorescence detection, normalised to actin levels and calculated as a percentage of HIF protein levels in saline-treated mice.

Antiangiogenic drugs reduce blood flow to tumours and thereby inhibit tumour growth by starving tumours of oxygen and nutrients. In so doing, antiangiogenic drugs can induce tumour hypoxia and up-regulate HIF-1α, a transcription factor that promotes tumour angiogenesis, invasion, and metastasis. This up-regulation of HIF-1α could underlie resistance to antiangiogenic therapy. We investigated whether the efficacy of antiangiogenic drugs could be improved by combining them with CRLX-101, which is a dual inhibitor of HIF-1a and topo-1.

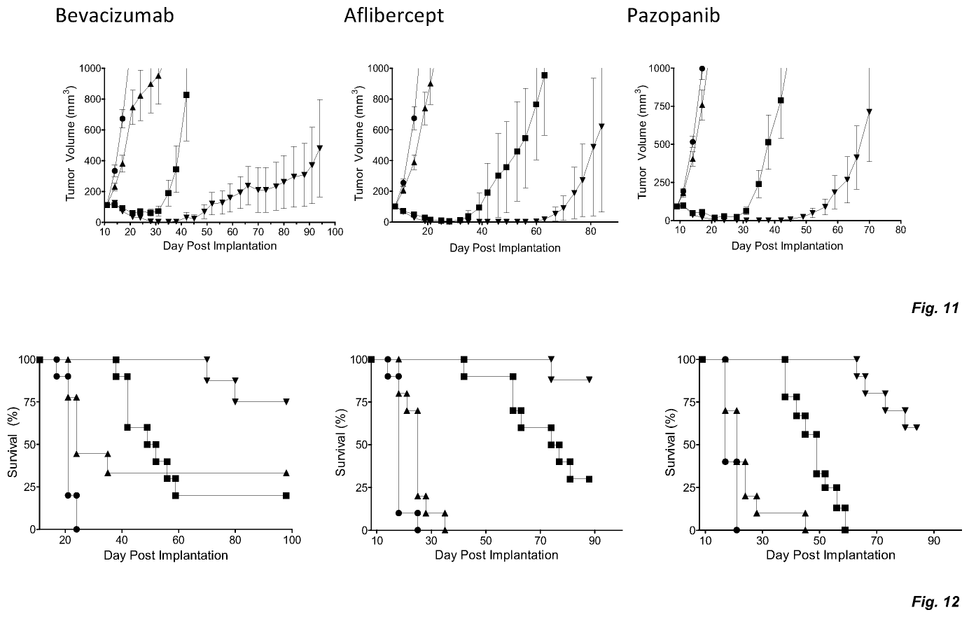

Three studies evaluated the antitumour activity of CRLX-101 in combination with the antiangiogenic drugs Bevacizumab, Aflibercept, or Pazopanib in athymic nude mice bearing human A2780 ovarian tumour implants. The combination of CRLX-101 with Bevacizumab, Aflibercept or Pazopanib was superior to either monotherapy when treated for three weeks, whether measured as tumour growth, survival or TFS. Combination therapy was well tolerated; BWL was not greater than 5%, and treatment-related clinical observations were normal during the studies. The tumour growth plots are illustrated in FIG. 11 (Antitumour Activity of CRLX-101 Combined with Bevacizumab, Aflibercept or Pazopanib in the Human A2780 Ovarian Tumour Model in Athymic Mice). The survival plots are illustrated in FIG. 12 (Survival of Human Ovarian A2780 Tumour-Bearing Mice Administered CRLX-101 in Combination with Bevacizumab, Aflibercept or Pazopanib).

Inhibition of the human A2780 ovarian xenografts by CRLX-101, Bevacizumab, Aflibercept, Pazopanib (▴) or the combination therapy versus vehicle (●). Mice were treated with vehicle or CRLX-101 at 5 mg/kg administered intravenously on Days 1, 8, and 15, Bevacizumab at 5 mg/kg administered intraperitoneally on Days 11, 14, 18, 21, 25 and 28, aflibercept at 25 mg/kg administered intraperitoneally on Days 8, 11, 15, 18, 22 and 25; Pazopanib at 150 mg/kg administered orally on Days 9-30 or the combination of CRLX-101 and either Bevacizumab, Aflibercept or Pazopanib at the same doses and schedules as described above. Tumour growth curves are means with bars indicating standard error. The CRLX-101 dose is in camptothecin equivalent. Endpoint size was set at 1000 mm³.

Inhibition of the human A2780 ovarian xenografts by CRLX-101, Bevacizumab, aflibercept, pazopanib (▴) or the combination therapy versus vehicle (●). Mice were treated with vehicle or CRLX-101 at 5 mg/kg administered intravenously on Days 1, 8, and 15, Bevacizumab at 5 mg/kg administered intraperitoneally on Days 11, 14, 18, 21, 25 and 28, Aflibercept at 25 mg/kg administered intraperitoneally on Days 8, 11, 15, 18, 22 and 25; Pazopanib at 150 mg/kg administered orally on Days 9-30 or the combination of CRLX-101 and either Bevacizumab, Aflibercept or Pazopanib at the same doses and schedules as described above. Tumour growth curves are means with bars indicating standard error. The CRLX-101 dose is in camptothecin equivalent. Endpoint size was set at 1000 mm³.

Consistent with the hypothesis that CRLX-101 can prevent the up-regulation of HIF-1α caused by antiangiogenic therapy, 4 studies evaluated HIF-1α protein levels after treatment with CRLX-101; the antiangiogenic drugs Bevacizumab, Aflibercept, Pazopanib, or Regorafenib (Stivarga®); and the combination in athymic nude mice bearing human A2780 ovarian tumour implants. In each study, CRLX-101 monotherapy inhibited tumour HIF-1α levels, while the antiangiogenic drugs Bevacizumab, Aflibercept, Pazopanib, and Regorafenib monotherapy increased tumour HIF 1 and the combination of CRLX-101 with each of the antiangiogenic drugs prevented the stimulation of HIF-1α by the antiangiogenic monotherapies, resulting in HIF-1α inhibition at levels similar to treatment with CRLX-101 as a monotherapy. The Table below summarises the results of HIF-1α inhibition by CRLX-101, Bevacizumab, Aflibercept, Pazopanib or Regorafenib monotherapy and the combination of CRLX-101 with Bevacizumab, Aflibercept, Pazopanib or Regorafenib as a percent of HIF-1α levels in untreated tumours.

TABLE
Inhibition of HIF-1α Protein Levels by CRLX-101
Alone or Combined with Bevacizumab,
Aflibercept, Pazopanib or Regorafenib
in the Human A2780 Ovarian Tumour
Model in Athymic Mice

| | | | | HIF-1Q |
|---|---|---|---|---|
| | | | Dose | % of Control |
| n | Treatment | Schedule | mg/kg | mean + SD |
| 3 | CRLX-101 | QW × 2 | 5 | 25 ± 15 |
| 3 | Bevacizumab | Days 1, 4, 8 | 5 | 127 ± 18 |
| 3 | CRLX-101 + | QW × 2 | 5 + 5 | 25 ± 4 |
| | Bevacizumab | Days 1, 4, 8 | | |
| 3 | CRLX-101 | QW × 2 | 5 | 25 ± 1 |
| 3 | Aflibercept | Days 1, 4, 8 | 25 | 235 ± 80 |
| 3 | CRLX-101 + | QW × 2 | 5 + 25 | 42 ± 11 |
| | Aflibercept | Days 1, 4, 8 | | |
| 3 | CRLX-101 | QW × 2 | 5 | 27 ± 26 |
| 3 | Pazopanib | QD × 8 | 150 | 226 ± 127 |
| 3 | CRLX-101 + | QW × 2 | 5 + 150 | 24 ± 24 |
| | Pazopanib | QD × 8 | | |
| 3 | CRLX-101 | QW × 2 | 5 | 41 ± 9 |
| 3 | Regorafenib | QD × 8 | 50 | 171 ± 4 |
| 3 | CRLX-101 + | QW × 2 | 5 + 50 | 31 ± 7 |
| | Regorafenib | QD × 8 | | |

Abbreviations: HIF-1α = hypoxia-inducible factor 1, alpha subunit;
n = number of animals in a group;
QW = once a week;
QD = daily.

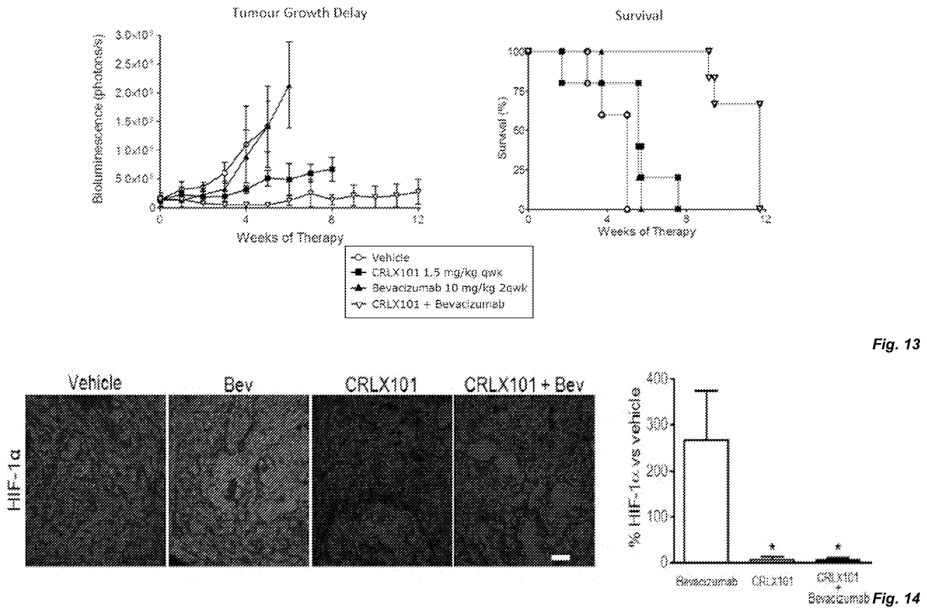

Synergistic antitumour activity of CRLX-101 in combination with the antiangiogenic drug Bevacizumab was also demonstrated in severe combined immunodeficient mice bearing highly metastatic orthotopic (intraperitoneal) human ovarian SKOV-3-13 tumour implants. SKOV-3-13 was developed as a sub-clone of the SKOV-3 cell line that gave rise to greater levels of peritoneal carcinomatosis.14 FIG. 13 (Tumour Growth and Survival of Orthotopic Human Ovarian SKOV-3-13 Tumour-Bearing Mice Administered CRLX-101 in Combination with Bevacizumab) shows that low-dose CRLX-101 combined with Bevacizumab led to superior efficacy versus either monotherapy treatment, measured by either tumour growth delay or survival. Tumours from the SKOV-3-13 model were also extracted following 3 weeks of treatment and processed for immunohistochemical expression of HIF 1α. As shown in FIG. 14 (HIF-1α Immunohistochemistry in Orthotopic Human Ovarian SKOV-3-13 Tumours Administered CRLX-101 in Combination with Bevacizumab), CRLX-101 was able to inhibit both HIF-1α expression compared with vehicle, even when combined with Bevacizumab.

Tumour growth delay and survival are greater for the combination of CRLX-101 plus Bevacizumab than either treatment alone or the sum of both individual treatments. Mice were dosed continuously throughout duration of the experiment at the dose level and frequency indicated in the figure legend. Tumour growth curves represent mean bioluminescence. Survival is indicated as the percentage of animals remaining on study. CRLX-101 dose is in camptothecin equivalent.

HIF-1α immunohistochemistry (light coloured) from tumours taken out of mice 1 week after 3 weeks of treatment as described above. Images were analysed and quantified, normalised to vehicle, and average staining from 4 mice is shown at right (* indicates p<0.05 vs. Bevacizumab monotherapy).

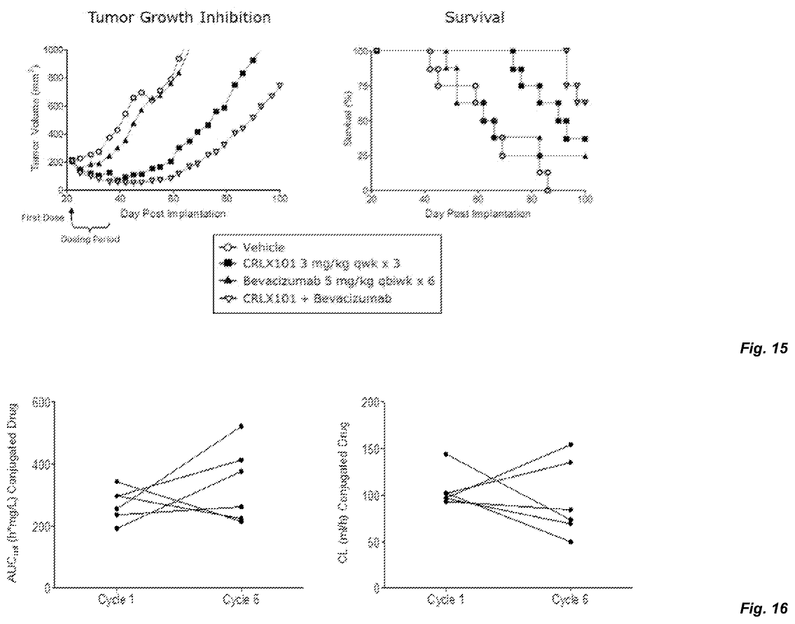

Antitumour activity of CRLX-101 in combination with the antiangiogenic drugs Bevacizumab was also demonstrated in athymic nude mice bearing human 786-O RCC tumour implants. The 786 O tumour cell line is representative of clear cell renal cell carcinoma (ccRCC) because it harbours a mutation in the von Hippel Lindau (VHL) tumour-suppressor protein and consequently has high levels of expression of HIF-2α. On the other hand, HIF-1α is not expressed in this cell line. Thus, combination studies with CRLX-101 in this model would suggest a role for inhibition of HIF-2α and not HIF-1α. FIG. 15 (Tumour Growth and Survival of Human Renal Cell Carcinoma 786-O Tumour-Bearing Mice Administered CRLX-101 in Combination with Bevacizumab) shows that CRLX-101 combined with Bevacizumab led to superior efficacy versus either monotherapy treatment, measured by either tumour growth delay or survival.

Tumour growth delay and survival are greater for the combination of CRLX-101 plus Bevacizumab than either treatment alone or the sum of both individual treatments. Tumour growth curves represent mean tumour volume. Survival is indicated as the percentage of animals remaining on study because mice bearing tumours that reached a predetermined cutoff size (1000 mm3) were removed from the experiment. CRLX-101 dose is in camptothecin equivalent.

### Example 7: Single Dose Pharmacokinetics in Sprague-Dawley Rats

Single dose pharmacokinetics of CRLX-101 were determined in non-tumour bearing female Sprague-Dawley rats. Doses of 0.88, 2.77, and 8.8 mg/kg were administered and plasma samples obtained at 0, 5, 15, 30 minutes, 1, 2, 4, 8, 12, 24, 48, 72, 96, and 120 hours. Plasma pharmacokinetic estimates for CRLX-101 are provided in the Table below.

TABLE
Total and Unconjugated CPT Pharmacokinetic Estimates in Sprague-Dawley Rats

| | | Total CPT | | | Unconjugated CPT | | |
|---|---|---|---|---|---|---|---|
| | | CRLX-101 | CRLX-101 | CRLX-101 | CRLX-101 | CRLX-101 | CRLX-101 |
| Parameters | Description | 8.8 mg/kg | 2.77 mg/kg | 0.88 mg/kg | 8.8 mg/kg | 2.77 mg/kg | 0.88 mg/kg |
| Dose Amount | μg | 1803.86 | 567.34 | 183.67 | 1803.86 | 567.34 | 183.67 |
| Dosage | μg/kg | 8761.94 | 2774.61 | 876.19 | 8761.94 | 2774.61 | 876.19 |
| E Half-life | hr | 16.40 | 14.33 | 8.71 | 7.964 | 6.137 | 2.617 |
| D Half-life | hr | 7.45 | 7.79 | 4.09 | 0.665 | 0.505 | 0.451 |
| A Half-life | hr | 0.48 | 0.57 | 0.34 | ~ | ~ | ~ |
| C initial (IV) | μg/mL | 104.47 | 29.92 | 10.30 | 2.0 | 0.5 | 0.1 |
| Cmax | μg/mL | 111.78 | 32.26 | 10.97 | 2.2 | 0.5 | 0.1 |
| Tmax | hr | 0.01 | 0.01 | 0.01 | 0.010 | 0.010 | 0.010 |
| CPT/ID initial | % | 82 | 74 | 80 | 1.61 | 1.15 | 0.73 |
| AUC∞ | μg-hr/mL | 698.39 | 205.09 | 57.97 | 6.3 | 1.3 | 0.2 |
| MRT | hr | 12.38 | 12.02 | 10.13 | 7.6 | 6.3 | 2.3 |
| Vc | mL | 21.28 | 23.37 | 21.88 | 1103.6 | 1328.1 | 1592.2 |
| Vd | mL | 75.34 | 70.49 | 48.91 | 4070.7 | 4672.7 | 4305.2 |
| Vd/kg | mL/kg | 365.96 | 344.72 | 234.24 | 19772.9 | 22852.3 | 20619.9 |
| Vss | mL | 39.41 | 40.98 | 39.40 | 2684.0 | 3338.0 | 2599.9 |
| CL | mL/hr | 3.18 | 3.41 | 3.89 | 354.228 | 527.667 | 1140.076 |
| CL/kg | mL/hr · kg | 15.46 | 16.68 | 18.63 | 1720.595 | 2580.592 | 5460.461 |

Abbreviations: CPT = 20(S)-camptothecin; Cmax = Maximum observed concentration; Tmax = Time of maximum observed concentration; CPT/ID initial = ratio of total detected CPT at t = 0 to the injected dose based on the assumption of blood volume = 64 ml/kg in rat (Diehl et al, J. Applied Toxicology, 2001, 21, 15-23); AUC∞ = total area under curve; MRT = Mean resident time; Vc = Apparent volume of the central compartment; Vd = Volume of distribution; Vd/kg = Vd normalised by animal weight; Vss = Volume of distribution at steady state; Cl = Systemic clearance; Cl/kg = Cl normalised by animal weight; Half-life from Vd and Cl = alternate calculation of half-life using V and Cl (0.693V/Cl)

These data indicate that the distribution of CRLX-101 is linear based on area under the curve (AUC) and probably follows first order pharmacokinetics. The data fit a 3-compartment model with a rapid phase, intermediate, and slow phase of distribution. The central volume of distribution is slightly larger than the predicted blood compartment of a rat at ˜100 mL/kg. The T½ of total CPT is ˜14 hours; however, data from the terminal phase is close to the minimum level of detection and as such may under or overestimate the true elimination rate. Plasma levels at 24 and 48 hours are presented in the Table below. The ratio of unconjugated to total CPT was similar to that observed in normal tissue ˜0.3%.

TABLE
Plasma Concentration of Total and Unconjugated CPT

| | | 24 hour | 48 hour |
|---|---|---|---|
| Plasma | Total CPT (ng/ml) | 21640 ± 1985.9 | 5862 ± 1314.4 |
| | Unconjugated CPT (ng/ml) | 72.1 ± 9.2 | 2.6 ± 3.3 |
| | Percent of unconjugated CPT (%) | 0.3% | 0.04% |

### Example 8: Multiple Dose Pharmacokinetics in Rats

TABLE
Pharmacokinetic Estimates of Total and Unconjugated CPT in Rats at Day 1 and Day 15

| | | Day 1 | | | | Day 15 | | | |
|---|---|---|---|---|---|---|---|---|---|
| | | | | Unconjugated | | | | Unconjugated | |
| | | Total CPT | | CPT | | Total CPT | | CPT | |
| Parameter | Units | M | F | M | F | M | F | M | F |
| CRLX-101 | mg/kg | 2.59 | 2.59 | 2.59 | 2.59 | 2.59 | 2.59 | 2.59 | 2.59 |
| dose | | | | | | | | | |
| Mean BW | kg | 0.223 | 0.154 | 0.223 | 0.154 | 0.272 | 0.183 | 0.272 | 0.183 |
| Adm dose | ng | 577570 | 398860 | 577570 | 398860 | 704480 | 473970 | 704480 | 473970 |
| Cmax | ng/ml | 25335 | 31957 | 1471 | 1320 | 34548 | 26632 | 1393 | 1079 |
| (observed) | | | | | | | | | |
| Tmax | hr | 1 | 0.25 | 2 | 4 | 0.25 | 0 | 1 | 0 |
| (observed) | | | | | | | | | |
| C0 | ng/ml | 29523 | 24603 | 1158 | 994 | 22880 | 23138 | 1019 | 1050 |
| (predicted) | | | | | | | | | |
| AUC0-inf | ng * hr/ | 392614 | 389908 | 27000 | 27765 | 479349 | 389078 | 19494 | 14739 |
| | mL | | | | | | | | |
| MRT | hr | 31.0 | 28.3 | 39.6 | 36.7 | 37.4 | 45.7 | 35.8 | 41.2 |
| T½, α | hr | 2.1 | 3.4 | 8.8 | 14.8 | 1.7 | 0.028 | 3.7 | 0.044 |
| T½, ß | hr | 25.1 | 23.6 | 40.9 | 45.3 | 27.4 | 31.7 | 29.3 | 28.6 |
| Cl | mL/hr | 1.47 | 1.02 | 21.4 | 14.4 | 1.47 | 1.22 | 36.1 | 32.2 |
| Cl | mL/hr/ | 6.59 | 6.62 | 96.0 | 93.5 | 5.40 | 6.67 | 133 | 176 |
| normalised | kg | | | | | | | | |
| V1 | mL | 19.6 | 16.2 | 499 | 401 | 30.8 | 20.5 | 691 | 451 |
| V1 | mL/kg | 87.9 | 105 | 2238 | 2604 | 113 | 112 | 2540 | 2464 |
| normalised | | | | | | | | | |
| Vss | mL | 45.7 | 29 | 848 | 527 | 55.0 | 55.6 | 1295 | 1323 |
| Vss | mL/kg | 205 | 188 | 3803 | 3422 | 202 | 304 | 4761 | 7230 |
| normalised | | | | | | | | | |
| V2 | mL | 26.1 | 12.7 | 349 | 126 | 24.2 | 35.1 | 603 | 872 |

Abbreviations: adm = administered; AUC = area under the curve; BW = body weight; Cl = systemic clearance; Cmax = maximum observed concentration; C0 = predicted concentration at time = 0; CPT = 20(S)-camptothecin; MRT = mean resident time; T½ = half-life; Tmax = time of maximum observed concentration; Vss = volume of distribution at steady state.

Data were fitted to a 2-compartment model using WIN NONLIN. At Day 15, the weighted clearance (Cl) was lower and the Vc, half-life beta (elimination) (T½ β), and mean residence time (MRT) were greater than observed at Day 1. Unconjugated CPT AUC, T½ β, were lower and weighted Cl, volume of distribution at steady state (Vss), were great than at Day 1. There were no meaningful differences in Vss or maximum concentration observed (Cmax) for bound CPT and MRT and Cmax for unconjugated CPT between Days 1 and 15. AUC for total (mostly conjugated) CPT were slightly (12%) higher at Day 15 than at Day 1 indicating a slight but nonsignificant accumulation of total CPT, however, the AUC for unconjugated CPT was about 50% lower at 15 days than that observed at Day 1. It will be appreciated that plasma values are highly variable in some of the animals.

### Example 9: Multiple Dose Pharmacokinetics in Beagle Dogs

Multiple-dose PK estimates were obtained in the dog sub-acute toxicology study at the 0.58 mg/kg dose at both Day 1 and Day 15. These data are presented in the Table below.

TABLE
Pharmacokinetic Estimates in Dogs at Day 1 and Day 15 Total CPT

| | | | | Day 15 | |
|---|---|---|---|---|---|
| | | Day 1 Total CPT | | Total CPT | |
| | | Males | Females | Males | Females |
| Parameter | Units | Mean | Mean | Mean | Mean |
| CRLX- | mg/kg | 0.58 | 0.58 | 0.58 | 0.58 |
| 101 dose | | | | | |
| BW | kg | 9.6 | 6.8 | 9.9 | 6.3 |
| Adm dose | ng | 5548667 | 3934667 | 5742000 | 3634667 |
| Cmax | ng/ml | 5299 | 5477 | 6981 | 5045 |
| (observed) | | | | | |
| Tmax | hr | 0.08 | 0 | 0.08 | 0.3 |
| (observed) | | | | | |
| C0 | ng/ml | 4955 | 5437 | 6693 | 4728 |
| (predicted) | | | | | |
| AUC0-inf | ng*hr/mL | 133269 | 136268 | 149316 | 123632 |
| MRT | hr | 38.0 | 33.1 | 34.7 | 37.1 |
| t1/2, α | hr | 7.14 | 0.57 | 1.01 | 3.74 |
| t1/2, β | hr | 32.2 | 23.1 | 24.6 | 27.6 |
| Cl | mL/hr | 41.7 | 28.9 | 38.7 | 29.6 |
| Cl | mL/hr/kg | 4.37 | 4.26 | 3.92 | 4.71 |
| normalised | | | | | |
| V1 | mL | 1131 | 729 | 875 | 781 |
| V1 | mL/kg | 119 | 107 | 88.9 | 124 |
| normalised | | | | | |
| Vss | mL | 1586 | 963 | 1342 | 1097 |
| Vss | mL/kg | 166 | 141 | 136 | 175 |
| normalised | | | | | |
| V2 | mL | 454 | 234 | 468 | 316 |

Abbreviations: adm = administered;
AUC = area under the curve;
BW = body weight;
Cl = systemic clearance;
Cmax = maximum observed concentration;
C0 = predicted concentration at time = 0;
CPT = 20(S)-camptothecin;
MRT = mean resident time;
T1/2 = half-life;
Tmax = time of maximum observed concentration;
Vss = volume of distribution at steady state.

There were no meaningful differences in the total CPT at Day 1 and Day 15 pharmacokinetic parameters in dogs. These data indicate no significant accumulation on a weekly dosing regimen in dogs. The T½ varied between 23 and 32 hours and the mean residence time varied between 33 and 38 hours. Unconjugated CPT pharmacokinetic data are provided in the Table below. Unconjugated CPT measured at Day 1 and Day 15 was similar and did not demonstrate any significant accumulation. Mean residence time varied between 33 and 47 hours and T½ varied between 20 and 34 hours.

TABLE
Pharmacokinetic Estimates in Dogs at Day 1 and Day 15 Unconjugated CPT

| | | Unconjugated CPT | | | |
|---|---|---|---|---|---|
| | | Day 1 | | Day 15 | |
| | | Males | Females | Males | Females |
| Parameter | Units | Mean | Mean | Mean | Mean |
| CRLX-101 dose | mg/kg | 0.58 | 0.58 | 0.58 | 0.58 |
| BW | kg | 9.6 | 6.8 | 9.9 | 6.3 |
| Adm dose | ng | 5548667 | 3934667 | 5742000 | 3634667 |
| Cmax (observed) | ng/ml | 418 | 260 | 269 | 237 |
| Tmax (observed) | hr | 0 | 0.7 | 0 | 0.08 |
| C0 (predicted) | ng/ml | 407 | 240 | 279 | 250 |
| AUC0-inf | ng*hr/mL | 3054 | 4776 | 4247 | 3427 |
| MRT | hr | 39.3 | 32.6 | 46.8 | 27.6 |
| t1/2, α | hr | 0.61 | 2.45 | 0.61 | 0.59 |
| t1/2, β | hr | 30.0 | 24.5 | 33.8 | 19.7 |
| Cl | mL/hr | 1909.3 | 866 | 1360.3 | 1061 |
| Cl normalised | mL/hr/kg | 198 | 127 | 138 | 169 |
| V1 | mL | 13777 | 16361 | 20706 | 14799 |
| V1 normalised | mL/kg | 1440 | 2418 | 2092 | 2354 |
| Vss | mL | 68708 | 28255 | 63699 | 29331 |
| Vss normalised | ml/kg | 7207 | 4129 | 6422 | 4669 |
| V2 | mL | 54931 | 11894 | 42993 | 14531 |

Abbreviations: adm = administered;
AUC = area under the curve;
BW = body weight;
Cl = systemic clearance;
Cmax = maximum observed concentration;
C0 = predicted concentration at time = 0;
CPT = 20(S)-camptothecin;
MRT = mean resident time;
T1/2 = half-life;
Tmax = time of maximum observed concentration;
Vss = volume of distribution at steady state.

### Example 10: Distribution

The tissue distribution of unconjugated and total CPT in animals treated with CRLX-101 is provided in the Table below.

TABLE
Tissue Concentration of Total and Unconjugated
CPT in Specific Organs

| Tissue | Form of CPT | 24 Hours | 48 Hours |
|---|---|---|---|
| Heart | Total CPT (ng/g) a | 6419.6 ± 880.9 | 2548.6 ± 565.6 |
| | Unconjugated CPT (ng/g) | 23.4 ± 21.3 | 4.5 ± 5.2 |
| | Percent of unconjugated | 0.4% | 0.2% |
| | CPT (%) b | | |
| Liver | Total CPT (ng/g) a | 12396.1 ± 2,197.1 | 6899.2 ± 1,975.4 |
| | Unconjugated CPT (ng/g) | 241.3 ± 131.9 | 121.4 ± 56.7 |
| | Percent of unconjugated | 1.9% | 1.8% |
| | CPT (%) b | | |
| Spleen | Total CPT (ng/g) a | 9647.9 ± 1799.8 | 7321.2 ± 2854.3 |
| | Unconjugated CPT (ng/g) | 45.4 ± 42.1 | 12.9 ± 16.7 |
| | Percent of unconjugated | 0.5% | 0.2% |
| | CPT (%) b | | |
| Lung | Total CPT (ng/g) a | 8683.8 ± 1072.8 | 3587.4 ± 901.3 |
| | Unconjugated CPT (ng/g) | 56.8 ± 43.2 | 10.5 ± 8.8 |
| | Percent of unconjugated | 0.7% | 0.3% |
| | CPT (%) b | | |
| Tumour | Total CPT (ng/g) a | 13901.3 ± 833.4 | 4175.3 ± 337.6 |
| | Unconjugated CPT (ng/g) | 183.3 ± 115.3 | 55.2 ± 22.1 |
| | Percent of unconjugated | 1.3% | 1.3% |
| | CPT (%) b | | |

Abbreviation: CPT = 20(S)-camptothecin.
a = ng/g = ng of CPT/gram of corresponding solid tissue
b = Percent of unconjugated CPT expressed as a percent average unconjugated CPT to average total CPT recovered in each tissue.

The ratio of tissue to plasma concentration for specific organs at 24 and 48 hours after dosing in nude mice is presented in the Table below.

TABLE
Ratio of Tissue to Plasma Unconjugated and Total CPT
24 and 48 hours after CRLX-101 Administration

| | | 24-Hour | 48-Hour |
|---|---|---|---|
| Tissue | Form of CPT | Sample | Sample |
| Heart | Total CPT | 30% | 43% |
| | Unconjugated CPT | 32% | 173% |
| Liver | Total CPT | 57% | 118% |
| | Unconjugated CPT | 335% | 4669% |
| Spleen | Total CPT | 45% | 125% |
| | Unconjugated CPT | 63% | 496% |
| Lung | Total CPT | 40% | 61% |
| | Unconjugated CPT | 79% | 404% |
| Tumour | Total CPT | 64% | 71% |
| | Unconjugated CPT | 254% | 2123% |

Abbreviation: CPT = 20(S)-camptothecin.

Plasma concentrations of total CPT were 21640±1958 ng/mL and 5862±1314 ng/mL at 24 and 48 hours, respectively. Plasma concentrations of unconjugated CPT were 72±9.2 ng/mL and 2.6±3.3 ng/mL at 24 and 48 hours, respectively. The tissue plasma gradient for total CPT at 24 hours was <100% for all major organs of interest including tumour indicating a slow phase of tissue distribution from the blood compartment. However, the tissue:plasma gradient for unconjugated CPT was greater than 100% possibly indicating active tissue degradation and release of unconjugated CPT. By 48 hours, total CPT tissue:plasma ratios are elevated some 50 150% from 24-hr values, possibly indicating ongoing tissue accumulation or increasing elimination. The tissue accumulation in the tumour may be representative of passive targeting of the nanoparticle through highly fenestrated neovascularised tumour tissue.

Integration of the plasma PK data and tissue distribution data and the IC50, data yielded some interesting pharmacodynamic and toxicokinetic observations. Of the 4 tumour cell lines studied in vitro for the determination of IC50, a range of inhibitory concentrations of 0.05 to 0.25 μM/L for conjugated CPT were determined. This concentration is 17.4 ng/mL for 0.05 μM/L and 87 ng/mL for 0.25 μM/L of total CPT. These data coupled with the tissue:plasma ratio indicate that for non-target organs, once plasma CRLX-101 concentrations drop below ˜45 to 217 ng/mL, tissue concentrations will fall below cytotoxic concentrations. In contrast, tumour concentrations of unconjugated CPT are higher than other tissues and increase to nearly 20 fold higher than plasma unconjugated CPT by 48 hours. The mechanism for this enhanced intra-tumour release of conjugated CPT is currently not known but may be related to higher concentrations of proteolytic enzymes found in inflammatory cells.

Metabolism and Toxicology

CRLX-101 was evaluated in vitro for metabolic stability, protein binding, red blood cell (RBC) partitioning, P450 inhibition, and plasma stability. CRLX-101 was shown to be stable in human, rat, and dog microsomes. CRLX-101 bound moderately to rat RBCs and did not significantly inhibit CYP450 enzymes.

### Example 11: Acute Pyramid Study in Sprague-Dawley Rats

The purpose of this study was to evaluate the potential toxicity of CRLX-101 following IV dose administration to Sprague-Dawley rats.

The test article, CRLX-101, a slightly yellow solid, was supplied by the University of Iowa. The test article was formulated into D5W, USP, for IV bolus administration. Thirty-six experimentally naïve Sprague-Dawley rats (18 males and 18 females), 7 weeks old and weighing 210 233 g (males)/131 193 g (females) at the outset of the study, received a single IV injection of CRLX-101.

Three male and 3 female rats were administered a single IV bolus injection of CRLX-101 at a dose level of 2.59, 5.30, 8.79, 13.19, or 17.45 mg/kg.

Two males, one treated with 13.19 mg/kg and one treated with 17.45 mg/kg were euthanised for humane reasons on Day 4 (3 days following dose administration) as a result of adverse clinical signs at these dose levels. Necropsy findings included mottled kidneys, a dark area filled with a light brown substance in the jejunum, cecum and colon, and a firm area in the jejunum for the male rat treated with 13.19 mg/kg; and mottled kidneys and dark areas in the spleen and stomach for the male rat treated with 17.45 mg/kg. Similar findings were also observed in a few male rats euthanised on Day 8, and included the presence of mottled kidneys in one male treated with 2.59 mg/kg and in one male treated with 17.45 mg/kg; an enlarged jejunum, and the presence of a light blue substance lining the cecum and colon in one male treated with 5.30 mg/kg. Based on the National Institutes of Health (NIH) criteria for animal toxicity, the NOAEL was considered to be 8.8 mg/kg for rats. The dose that causes severe toxicity was determined to be 13.19 mg/kg in males and greater than 17.45 mg/kg in females.

In view of these findings, the NOAEL of CRLX-101 was determined to be 8.79 mg/kg administered intravenously on a single occasion and was well tolerated up to 7 days following treatment in the male and female Sprague-Dawley rat.

The STD of CRLX-101 administered intravenously on a single occasion was 13.19 mg/kg in male and 17.45 mg/kg in female Sprague-Dawley rats.

### Example 12: Sub-Acute Toxicology in Sprague-Dawley Rats

The purpose of this study was to evaluate the potential toxicity of CRLX-101 following IV administration, once weekly for 3 weeks, to Sprague-Dawley rats.

The test article, CRLX-101, a slightly yellow solid, was supplied by the University of Iowa. The test article was formulated into D5W, USP, for IV bolus administration to Sprague-Dawley rats. One hundred twenty-six experimentally naïve rats (63 males and 63 females), 7-weeks-old and weighing 157 243 g (males)/136 174 g (females) at the outset of the study, received a single weekly IV bolus injection of the vehicle control (D5W), the polymer control article (Poly-CD-PEG) or CRLX-101.

Nine male and 9 female rats received an IV injection of CRLX-101 once weekly for a total of 3 weeks (a total of 3 injections), at doses of 2.59, 7.76, or 11.64 mg/kg.

One male treated with 7.76 mg/kg was found dead on Day 4 (3 days following dose administration). There were no clinical signs observed prior to death (Days 1 4), and gross necropsy findings were not observed for this animal. Necrosis and/or degeneration, considered to have occurred as a result of test article administration, were noted in multiple organs including the large and small intestine, liver, urinary bladder, spleen, heart, brain and bone marrow. Vascular lesions (fibrinoid necrosis) were noted concurrently in the liver. Additionally, attempts at cellular regeneration were noted in the liver, intestine, and urinary bladder. The majority of organ specific toxicity was observed at the 11.6 mg/kg dose.

Treatment-related clinical signs included a thin body condition, pale extremities, red periorbital staining and brown urogenital staining. These clinical signs were only observed in a few animals (one male and one female) treated with 11.64 mg/kg. Treatment-related reduced mean weight gains were recorded for males and females treated with 11.64 mg/kg midweek following each dose (Days 4, 11, and 18). On each day of treatment following the first dose (Days 8 and 15), on Day 21 (1 week after the last dose), and during the recovery period (Days 25 and 28), the mean weight gains of male and female rats were generally comparable to or higher than the mean weight gains of the concurrent control groups, suggesting that the weight losses were reversible within 4 days to 1 week after treatment.

Similar effects on weight gains were recorded for male rats treated with 7.76 mg/kg, however, the magnitude of the changes were smaller than those recorded for the 11.64 mg/kg-treated group. Reduced weekly food intake values were recorded for males treated with 11.64 mg/kg 1 week following the first and second doses (Days 8 and 15). No other treatment-related differences were observed on food intake during the study.

Reversible treatment-related haematology findings recorded on Day 22 included the following: reduced erythrocyte counts and haemoglobin levels for males and females treated with 11.64 mg/kg; reduced mean haematocrit and elevated platelet counts for males treated with 11.64 mg/kg; and elevated reticulocyte counts for males and females treated with 7.76 mg/kg or 11.64 mg/kg. None of the haematology findings remained apparent on Day 29.

Gross necropsy findings were exclusively observed in animals treated with 11.64 mg/kg at the time of each scheduled euthanasia (Days 22 and 29), and were probably attributable to the administration of CRLX-101 at a dose of 11.64 mg/kg. Kidney findings were the most frequently observed necropsy findings and included the following: tan discolorations in the kidneys of two males on Day 29; white foci in the kidneys of one male on Day 22 and in one female on Day 29; a distorted kidney in one male on Day 29. Other gross necropsy findings included the adherence of the ileum to the cecum, and enlarged mesenteric lymph nodes in one male on Day 22; an enlarged spleen in one male on Day 22; and an enlarged or distorted spleen in two males on Day 29. The single organ weight finding recorded on Days 22 and 29 included a nonreversible increase in spleen weight for male rats treated with 2.59, 7.76, or 11.64 mg/kg and for female rats treated with 11.64 mg/kg.

Test article-related brain lesions were observed on Day 22 in animals treated with 11.64 mg/kg. These lesions were suggestive of vascular compromise, and consisted of focal areas of liquefactive necrosis or cavitation, accompanied by infiltration of foamy macrophages (gitter cells). Minimal, focal areas of gliosis and vacuolation were also observed at a dose of 11.64 mg/kg. Testicular lesions were also predominantly observed on Day 22 at a dose of 11.64 mg/kg, and consisted of minimal to mild, unilateral to bilateral necrosis of seminiferous tubule germinal epithelium, and minimal to mild, unilateral to bilateral depletion of germinal epithelium, accompanied by spermatic giant cells. These brain and testis lesions and severity of lesions were of a lower incidence and severity by Day 29. However, the reduced incidence and severity of lesions in the brain were not considered a result of recovery, but a variation in lesion detection resulting from a number of variables, including actual lesion occurrence or sectioning. In the testis, improvement in lesion severity and incidence may have resulted from the regenerative capabilities of the germinal cell population, assuming spermatogonia remained viable.

Non-reversible treatment-related heart and kidney lesions were observed on Day 22 predominantly in animals treated with 7.76 or 11.64 mg/kg. Minimal to mild, focal or multifocal myofiber degeneration and/or necrosis, and mild to moderate renal tubule degeneration and/or necrosis or infarction, were observed in animals from each of these dose levels. On Day 29, these heart and kidney lesions remained similar in incidence but progressed to chronic scarring for each of the 7.76 and 11.64 mg/kg dose groups.

Non-reversible treatment-related lesions in the pancreas, consisting of minimal and focal to moderate and widespread acinar cell degeneration (accompanied by chronic inflammation in the majority of cases), and characterised by necrosis and loss of acini, were observed on Day 22 of 50% of animals treated with 11.64 mg/kg. The presence of minimal periglandular or interlobular inflammation (without degeneration) was also observed on Day 22 in a few animals treated with 7.76 mg/kg. These lesions remained apparent on Day 29 in several animals treated with 7.76 mg/kg or 11.64 mg/kg.

Non-reversible treatment-related liver findings observed on Day 22 included the presence of minimally increased sinusoidal macrophages (histiocytes) at a dose of 2.59 mg/kg and mildly increased sinusoidal macrophages at a dose of 11.64 mg/kg. This change was also accompanied by minimal to mild vacuolation of sinusoidal macrophages in all affected animals. Additionally, in a few animals treated with 2.59 mg/kg, in all animals treated with 7.76 mg/kg and in one animal treated with 11.64 mg/kg, sinusoidal macrophages were not increased but were vacuolated. The presence of extramedullary haematopoiesis was also noted in the majority of animals treated with 11.64 mg/kg. On Day 29, a minimal increase in sinusoidal macrophage remained apparent in one animal treated with 11.64 mg/kg, and minimal vacuolation of sinusoidal macrophages remained apparent in two animals treated with 2.59 mg/kg, five animals treated with 7.76 mg/kg and in all animals treated with 11.64 mg/kg.

Non-reversible treatment related as well as polymer control-related lesions occurred in the spleen and at the injection site both on Day 22 and on Day 29. Spleen findings, consisting of reticuloendothelial cell hypertrophy, characterised by increased prominence of red pulp macrophages resulting from cytoplasmic microvesicular or macrovesicular vacuolation, were present in a minimum of two polymer control animals, and in a minimum of 4, 5, and 8 animals treated with 2.59, 7.76, and 11.64 mg/kg, respectively. An additional spleen finding, which was exclusively observed at a dose of 11.64 mg/kg in a minimum of 6 animals, consisted of minimal to mild depletion of lymphocytes in the marginal zone. A minimal to marked increase in extramedullary haematopoiesis was also observed in one polymer control animal (on Day 22 only) and in a minimum of 4 animals treated with 11.64 mg/kg. Injection site findings included minimal to mild, subcutaneous and/or perivascular subacute inflammation in one polymer control animal on each of Days 22 and 29, and in a minimum of one, three and two animals treated with 2.59, 7.76 of 11.64 mg/kg, respectively on each of Days 22 and 29.

Treatment-related findings in the GI tract and urinary bladder were exclusively observed in the male found dead on Day 4 (following a single dose at 7.76 mg/kg on Day 1). These lesions included marked full thickness haemorrhagic necrosis in the cecum and colon, and minimal crypt necrosis and regeneration in the duodenum and jejunum. Lesions in the urinary bladder consisted of mild, multifocal epithelial necrosis, and mild, diffuse regenerative hyperplasia, accompanied by mild, multifocal submucosal haemorrhage.

In view of these findings, the NOAEL of CRLX-101 administered intravenously once weekly for 3 consecutive weeks to male or female Sprague-Dawley rats was not determined.

The STD of CRLX-101 administered intravenously once weekly for 3 consecutive weeks to male or female Sprague-Dawley rats was 7.76 mg/kg.

### Example 13: Acute Dose Ranging Study in Beagle Dogs

The purpose of this study was to evaluate the potential toxicity of CRLX-101 following IV dose administration to Beagle dogs.

The test article, CRLX-101, a slightly yellow solid, was supplied by the University of Iowa. The test article was formulated into D5W, United States Pharmacopeia (USP) grade, for IV bolus administration to dogs. Two experimentally naive dogs (1 male and 1 female), approximately 5.5 6 months old and weighing 10.5 kg (male) and 5.3 kg (female) at the outset of the study, received a single weekly IV bolus injection of CRLX-101 at escalating doses of 0.78, 1.55 and 2.59 mg/kg, respectively. The female dog received an additional IV injection of CRLX-101 at a dose of 3.88 mg/kg 1 week following the administration of the 2.59 mg/kg dose.

One male and one female Beagle dog received a single weekly IV bolus injection of CRLX-101 at escalating doses of 0.78, 1.55, and 2.59 mg/kg, respectively. The female dog received an additional IV injection of CRLX-101 at a dose of 3.88 mg/kg 1 week following the administration of the 2.59 mg/kg dose. The male dog was euthanised on the seventh day following treatment at 2.59 mg/kg, and the female dog was euthanised on the third day following treatment at 3.88 mg/kg. Thinness, an abnormal gait/stance, decreased activity, prostration (described as lateral recumbency or curled up into a ball) were observed shortly prior to euthanasia in both animals. A severe weight loss (1.1 to 1.4 kg) and a prolonged lack of food intake (for a minimum of 3 consecutive days) were recorded at the time of euthanasia for both animals. Haematology and serum chemistry results obtained from the male dog shortly prior to euthanasia revealed a reduced total leukocyte count (with an accompanying reduced neutrophil count); reduced absolute lymphocyte and monocyte counts; elevated relative lymphocyte and eosinophil counts; a severely reduced erythrocyte count, along with reduced haemoglobin and haematocrit levels, reduced platelet and reticulocyte counts; reduced sodium, potassium and phosphorus levels; and elevated cholesterol values. Gross necropsy findings observed in both animals included the presence of dark red areas lining the stomach, cecum, colon, and rectum; dark firm areas in the right lobes of the lungs in the male dog; and dark red mesenteric lymph nodes in the female dog.

In view of these findings, doses of 0.78 and 1.55 mg/kg administered intravenously once weekly were well tolerated in the male and female Beagle dog.

The STD of CRLX-101 administered intravenously once weekly was 2.59 mg/kg in the male and 3.88 mg/kg in the female Beagle dog.

### Example 14: Sub-Acute Toxicology in Beagle Dogs

The purpose of this study was to evaluate the potential toxicity of CRLX-101 following IV administration, once weekly for 3 weeks, to Beagle dogs.

According to the protocol, 40 Beagle dogs (20 males and 20 females) were randomly divided into 5 groups and treated with CRLX-101, vehicle (D5W, USP), or polymer (Poly-CD-PEG) via IV bolus injection on each of Days 1, 8, and 15.

Eight Beagle dogs (4 males and 4 females) per group received an IV injection of CRLX-101 once weekly for a total of 3 weeks (a total of 3 injections), at doses of 0.58, 1.55, or 2.33 mg/kg.

All animals treated with 2.33 mg/kg (4 males, 4 females) were euthanised prior to the scheduled days of euthanasia (Days 22 and 29), as a result of adverse effects of CRLX-101 at this dose level. Of these animals, 3 were sacrificed in a moribund condition: 1 male was euthanised on Day 8 after receiving a single dose on Day 1; and 1 male and 1 female were euthanised on Days 10 and 11, respectively, after receiving 2 doses on Days 1 and 8. The other dogs within this dose group (2 males and 3 females) were euthanised on Days 12 and 13, respectively. Clinical signs indicative of overall health deterioration were observed in all of these animals at the time of euthanasia. These clinical signs included a combination of several of the following in each animal: a thin body condition, decreased activity, an abnormal gait/stance, weakness, pale gingiva, emesis, and abnormal fecal output/abnormal feces. A weight loss of 1.5 kg or greater was recorded for all male dogs except one (weight loss of 0.5 kg), and a weight loss of 0.7 kg or greater was recorded for all females. Reduced daily food intake values were also recorded for all 8 dogs on a minimum of 50% or more days on study prior to euthanasia.

Blood samples obtained shortly prior to euthanasia in each of these animals revealed the presence of reduced total leukocyte counts, accompanied by reduced neutrophil and elevated lymphocyte counts, and reduced monocyte, eosinophil and reticulocyte counts for the majority of male or female dogs.

Gross necropsy findings were observed in 3 of the 8 animals euthanised prior to the completion of the study, and was indicative of GI irritation: (dark red striations/mucosa in the cecum, colon, ileum, jejunum and/or stomach; red foci in the mesenteric lymph nodes). Histopathological findings at a dose of 2.33 mg/kg included epithelial necrosis, mucosa and/or epithelial atrophy and regeneration at multiple levels of the GI tract (from the oesophagus to the rectum), lymphoid depletion in multiple lymphoid organs, and thymic atrophy in all of the 8 dogs treated with 2.3 mg/kg. Reticuloendothelial activation, as evidenced by the presence of sinusoidal microgranulomas, was present in the majority of dogs (7 of 8), and diffuse atrophy of the hair follicle germinal epithelium was present in a few dogs (2 of 8) treated with 2.33 mg/kg. A moderate to marked bone marrow hypocellularity and/or depletion of granulocytic or erythrocytic cell lines was also observed in all animals treated with 2.33 mg/kg. An increase in cytoplasmic basophilia, depletion of secretory content and/or acinar cell atrophy in multiple glands, and zymogen depletion in the pancreas were also observed in animals treated with 2.33 mg/kg. The majority of organ specific toxicity was observed at 2.33 mg/kg and was predominantly associated with GI mucositis and inflammatory changes.

None of the animals treated with 0.58 or 1.55 mg/kg were sacrificed moribund or found dead during the study. Treatment-related clinical signs were not observed in males or females at doses of 0.58 or 1.55 mg/kg. Treatment-related mean weight losses were observed midweek after each dose (Days 4, 11 and 18) for males and females treated with 1.55 mg/kg, and effects on weight gain were more pronounced in males than in females at this dose level. Effects on food consumption correlated with the weight losses observed in dogs treated with 1.55 mg/kg, the lowest food intake being observed midweek during treatment.

Treatment-related decreases in total leukocyte count, erythrocyte count, haemoglobin, and haematocrit were observed on Day 22 for males and females treated with 1.55 mg/kg. A reduced platelet count and neutrophil count (absolute and relative counts), accompanied by an elevated lymphocyte count (absolute count only) were also recorded on Day 22 for male dogs treated with 1.55 mg/kg. None of these haematological changes remained apparent by Day 29.

No treatment-related effects on coagulation, clinical chemistry, or urinalysis parameters were observed on Days 22 or 29 for animals treated with 0.58 or 1.55 mg/kg.

No treatment-related gross necropsy findings were observed on Days 22 or 29 for male or female dogs treated with 0.58 or 2.33 mg/kg. An apparent treatment-related decrease in thymus weight (absolute, thymus-to-body, and thymus-to-brain weight ratios) was recorded on Day 22 for female dogs treated with 1.55 mg/kg. This finding was no longer apparent by Day 29.

Histopathological findings at doses of 0.58 or 1.55 mg/kg were only observed on Day 22 and included moderate hypocellularity in the bone marrow of 2 of 6 animals treated with 1.55 mg/kg; minimal to mild multifocal to diffuse depletion of the zymogen granules in the pancreas in 4 of 6 animals treated with 1.55 mg/kg and in 1 of 6 animals treated with 0.58 mg/kg; lymphoid depletion of the periarterial lymphoid sheaths (white pulp) in the spleen, of 1 of 6 animals treated with 1.55 mg/kg; and minimal lymphoid depletion in the mandibular lymph nodes of 1 of 6 animals treated with 0.58 mg/kg.

Based on these findings, a NOAEL could not be established in male and female Beagle dogs. However, a dose of 0.58 and 1.55 mg/kg administered once weekly on three separate occasions did not result in any prolonged apparent treatment-related findings 2 weeks after the last dose (day 29).

The STD of CRLX-101 administered intravenously once weekly for 3 consecutive weeks was 2.33 mg/kg in male and female Beagle dogs.

### Example 15: Summary of HED Estimates of the Severely Toxic Dose and Starting Dose Determination

The estimates of the HED from rat and dog studies is provided in the Table below. The most sensitive species appears to be the dog. The estimated human STD used to calculate the starting dose for the clinical study is 42 mg/m².

TABLE
Human Equivalent Dose Estimates for CRLX-101 STD

| | STD mg/ | HED | Supported |
|---|---|---|---|
| Species | kg/week | mg/m2 | Starting Dose * |
| Sprague-Dawley | 13.9 mg/kg | 90 mg/m2 | n/a |
| Rats (single dose) | | | |
| Sprague-Dawley Rats | 7.8 mg/kg | 51 mg/m2 | 5.1 mg/m2 |
| (3 weekly doses) | | | |
| Beagle Dogs | 2.59 mg/kg | 46 mg/m2 | n/a |
| (single dose) | | | |
| Beagle Dogs | 2.33 mg/kg | 42 mg/m2 | 7.00 mg/m2 |
| (3 weekly doses) | | | |

Abbreviations:
HED = human equivalent dose;
STD = severely toxic dose.
* DeGeorge, J.J, Ahn, C.-H., et al. (1998), Cancer Chemother Pharmacol 41; 173-185. 1/10 of STD in rodent species, 1/6 of STD in non-rodent species.

Based on the toxicology analysis a starting dose of 6.0 mg/m² was selected as the starting dose for the human clinical trial.

Effects in Humans

### Example 16: Pharmacokinetics and Drug Metabolism

In the Phase 1 dose escalation cohort, blood samples for determination of conjugated and unconjugated (released) CPT plasma concentration were collected pre-dose and post dose from 15 minutes to 336 hours (pre-second dose) for Cycles 1 and 6. A pre-dose sample was collected on Days 1 and 15 of every other cycle. In the Phase 2a MTD cohort samples were collected pre-dose and throughout Cycles 1 and 6 and pre-dose on Days 1 and 15 of every other cycle.

Following IV administration of CRLX-101 at 6, 12, 15 and 18 mg/m² (Phase 1 cohort) and 15 mg/m² only for those in the Phase 2a cohort, evidence of systemic plasma exposure to both polymer-conjugated and unconjugated CPT was observed in all subjects. Polymer-conjugated CPT plasma concentrations increased sharply following IV infusion of CRLX-101 with mean Cmax values ranging from 3580 to 8780 ng/mL over the dose range evaluated. The Cmax for conjugated CPT was reached within the first 2 hours for most subjects. Unconjugated CPT plasma concentrations increased gradually after the IV infusion of CRLX-101 with mean Cmax values ranging from 116 to 351 ng/mL over the dose range evaluated. Mean time to maximum observed concentration (Tmax) for unconjugated CPT ranged from 17.7 to 24.5 hours over the dose range evaluated. The prolonged Tmax values are consistent with a gradual and slow release of CPT from the polymer conjugate. Polymer-conjugated and unconjugated CPT exposure (as assessed by mean Cmax and AUC between the time of dose and the last time point at 24 hours [AUCall]) increased in a dose-related fashion over the 6 to 15 mg/m² dose range. Unconjugated CPT exposure also increased over the 15 to 18 mg/m² dose range; however, polymer-conjugated CPT exposure did not show further increase over the 15 to 18 mg/m² dose range. An explanation for this is uncertain. However, it was noted that there was a slight decrease in the Cycle 1 and Cycle 6 mean Cmax values for the 15 mg/m² dose group during the MTD phase compared to the 15 mg/m² dose group during the dose escalation phase. These lower mean values resulted in a reasonably proportional increase in exposure as dose increased from 15 to 18 mg/m². It is unknown if this lower exposure is a result of the shorter infusion time during the MTD phase of the study or the increase in sample size.

Over the full dose range, the observed increases in Cmax and AUCall were reasonably proportional to dose for polymer-conjugated and unconjugated CPT. Comparison of polymer-conjugated to unconjugated CPT exposure (as assessed by AUCall) revealed a ˜11-fold increase in exposure for conjugated CPT relative to unconjugated CPT. Despite slight differences, T½ values appeared to be dose-independent over the dose range evaluated with individual subject T½ values ranging from 19.9 to 42.0 hours and 27.5 to 111 hours for the conjugated and unconjugated species, respectively. The prolonged T½ of the unconjugated CPT is likely related to the slow release of CPT from polymer conjugate distributed to tissues; however, the T½ values for unconjugated CPT should be interpreted with caution due to the limited terminal phase time points. Mean clearance and volume of distribution values for the conjugated CPT over the dose range evaluated were dose-independent and ranged from 0.0914 to 0.132 L/hr and 2.33 to 4.63 L/hr, respectively. Volume of distribution values for the conjugated CPT suggests this material was retained within the vasculature and highly perfused tissues. The majority of subjects receiving CRLX-101 at 15 mg/m² or 18 mg/m² on a bi-weekly schedule had measurable levels of unconjugated CPT in plasma at 14 days post CRLX-101 administration; however, these values represented less than 3.1% of the respective mean Cmax values for each dose level. The lower dose group (12 mg/m²) on a bi-weekly schedule did not have any measurable levels of unconjugated CPT in their plasma by 14 days post CRLX-101 administration.

These results indicate that in order to avoid significant carry-over of unconjugated plasma CPT from one dose to the next, a dosing interval greater than 1 week may be required. No polymer-conjugated CPT was detectable in plasma at 14 days post administration of CRLX-101 for the 18 mg/m², 15 mg/m², or 12 mg/m² dose groups on a bi weekly schedule during the dose escalation phase. However, a small number of subjects did have measurable conjugated CPT exposure during the MTD evaluation phase at 14 days post administration of CRLX-101 for the 15 mg/m² dose group, but these values represent less than 0.2% of Cmax. The Tables below display plasma conjugated and unconjugated data following CRLX-101 infusion.

TABLE
Group Mean Conjugated CPT Pharmacokinetic Summary Data

| Dose Group | Cmax | Cmax/Dose | HLλz | AUCall | AUCINFobs | AUCINFobs/Dose | Clobs | VSSobs |
|---|---|---|---|---|---|---|---|---|
| Statistic | (μg/L) | (m2 * μg/L/mg) | (hr) | (hr * mg/L) | (hr * mg/L ) | (hr * m2 * mg/L/mg) | (L/hr) | (L) |
| 6 mg/m2 | | | | | | | | |
| N | 6 | 6 | 6 | 6 | 6 | 6 | 6 | 6 |
| Mean | 3580 | 596 | 28.9 | 110 | 116 | 19.3 | 0.0992 | 3.34 |
| SD | 457 | 76.2 | 4.99 | 26.2 | 22.5 | 3.74 | 0.0222 | 0.585 |
| Geometric Mean | 3550 | 592 | 28.5 | 108 | 114 | 19.0 | 0.0971 | 3.30 |
| 12 mg/m2 | | | | | | | | |
| N | 6 | 6 | 6 | 6 | 6 | 6 | 6 | 6 |
| Mean | 5620 | 468 | 30.2 | 178 | 188 | 15.7 | 0.132 | 4.63 |
| SD | 872 | 72.7 | 5.20 | 42.9 | 38.2 | 3.19 | 0.0236 | 1.07 |
| Geometric Mean | 5560 | 463 | 29.9 | 174 | 185 | 15.4 | 0.130 | 4.53 |
| 15 mg/m2 | | | | | | | | |
| N | 6 | 6 | 6 | 6 | 6 | 6 | 6 | 6 |
| Mean | 8770 | 555 | 31.5 | 285 | 291 | 18.5 | 0.103 | 3.52 |
| SD | 1830 | 134 | 1.50 | 69.7 | 71.1 | 5.21 | 0.0454 | 1.60 |
| Geometric Mean | 8600 | 539 | 31.5 | \|278 | 284 | 17.8 | 0.0954 | 3.26 |
| 18 mg/m2 | | | | | | | | |
| N | 6 | 6 | 6 | 6 | 6 | 6 | 6 | 6 |
| Mean | 8780 | 488 | 33.5 | 256 | 273 | 15.1 | 0.122 | 4.62 |
| SD | 1650 | 91.4 | 6.92 | 51.3 | 42.5 | 2.36 | 0.0311 | 1.26 |
| Geometric Mean | 8660 | 481 | 32.9 | 252 | 270 | 15.0 | 0.118 | 4.45 |

Abbreviations: AUC = area under the curve; Clobs = observed systemic clearance; Cmax = maximum concentration; CPT = 20(S) camptothecin; HL = half-life; Tmax = time of maximum observed concentration; Vssobs = observed volume of distribution at steady state.

TABLE
Group Mean Unconjugated CPT Pharmacokinetic Summary Data

| Dose Group | Cmax | Cmax/Dose | Tmax | HLλz | AUCall | AUCINFobs | AUCINFobs/Dose |
|---|---|---|---|---|---|---|---|
| Statistic | (μg/L) | (m2 * μg/L/mg) | ( hr ) | (hr) | (hr * mg/L) | (hr * mg/L) | (hr * m2 * mg/L/mg) |
| 6 mg/m2 | | | | | | | |
| N | 6 | 6 | 6 | 5 | 6 | 5 | 5 |
| Mean | 116 | 19.3 | 17.7 | 48.4 | 9.98 | 13.2 | 2.20 |
| SD | 61.1 | 10.2 | 10.3 | 16.1 | 6.32 | 7.04 | 1.17 |
| Geometric Mean | 105 | 17.4 | 12.9 | 46.2 | 7.96 | 12.0 | 1.99 |
| 12 mg/m2 | | | | | | | |
| N | 6 | 6 | 6 | 6 | 6 | 6 | 6 |
| Mean | 203 | 16.9 | 19.3 | 50.1 | 13.7 | 16.6 | 1.38 |
| SD | 62.6 | 5.21 | 7.47 | 30.6 | 13.05 | 3.60 | 0.300 |
| Geometric Mean | 196 | 16.4 | 17.7 | 44.7 | 13.4 | 16.3 | 1.36 |
| 15 mg/m2 | | | | | | | |
| N | 6 | 6 | 6 | 6 | 6 | 6 | |
| Mean | 268 | 17.8 | 24.5 | 43.3 | 23.6 | 25.4 | 1.69 |
| SD | 92.5 | 6.17 | 1.14 | 7.86 | 7.94 | 8.47 | 0.564 |
| Geometric Mean | 256 | 17.1 | 24.5 | 42.7 | 22.4 | 24.1 | 1.61 |
| 18 mg/m2 | | | | | | | |
| N | 6 | 6 | 6 | 5 | 6 | 5 | 5 |
| Mean | 351 | 19.5 | 22.8 | 41.9 | 27.4 | 31.5 | 1.75 |
| SD | 146 | 8.10 | 6.47 | 10.6 | 9.82 | 10.9 | 0.607 |
| Geometric Mean | 327 | 18.2 | 21.6 | 40.9 | 26.0 | 29.9 | 1.66 |

Abbreviations: AUC = area under the curve; Clobs = observed systemic clearance; Cmax = maximum concentration; CPT = 20(S) camptothecin; HL = half-life; Tmax = time of maximum observed concentration; Vssobs = observed volume of distribution at steady state.

TABLE
Group Mean Conjugated CPT Pharmacokinetic Summary Data- MTD Evaluation at 15 mg/m²,
Cycle 1 and Cycle 6

| Dose Group | Cmax | Cmax/Dose | HLλz | AUCall | AUCINFobs | AUCINFobs/Dose | Clobs | VSSobs |
|---|---|---|---|---|---|---|---|---|
| Statistic | (μg/L) | (m2 * μg/L/mg) | (hr) | (hr * mg/L) | (hr * mg/L) | (hr * m2 * mg/L/mg) | (L/hr) | (L) |
| Cycle 1 | | | | | | | | |
| N | 36 | 36 | 36 | 36 | 36 | 36 | 36 | 36 |
| Mean | 8260 | 551 | 27.9 | 300 | 306 | 20.4 | 0.0914 | 2.42 |
| SD | 1300 | 87.0 | 2.93 | 60.6 | 57.8 | 3.86 | 0.0233 | 0.702 |
| Geometric | 8150 | 543 | 27.7 | 1293 | 300 | 20.0 | 0.0885 | 2.32 |
| Mean | | | | | | | | |
| Cycle 6 | | | | | | | | |
| N | 6 | 6 | 6 | 6 | 6 | 6 | 6 | 6 |
| Mean | 7190 | 479 | 27.5 | 323 | 335 | 22.3 | 0.0943 | 2.33 |
| SD | 1800 | 120 | 3.77 | \|128 | 122 | 8.13 | 0.0411 | 1.25 |
| Geometric | 6980 | 465 | 27.2 | 303 | 317 | 21.2 | 0.0873 | 1.96 |
| Mean | | | | | | | | |

Abbreviations: AUC = area under the curve; Clobs = observed systemic clearance; Cmax = maximum concentration; CPT = 20(S) camptothecin; HL = half-life; Tmax = Time of maximum observed concentration; Vssobs = observed volume of distribution at steady state.

TABLE
Group Mean Unconjugated CPT Pharmacokinetic Summary Data MTD Evaluation at
15 mg/m², Cycle 1 and Cycle 6

| Cycle | Cmax | Cmax/Dose | Tmax | HLλz | AUCall | AUCINFobs | AUCINFobs/Dose |
|---|---|---|---|---|---|---|---|
| Statistic | (μg/L) | (m2 * μg/L/mg) | (hr) | (hr) | (hr * mg/L) | (hr * mg/L) | (hr * m2 * mg/L/mg) |
| Cycle 1 | | | | | | | |
| N | 36 | 36 | 36 | 11 | 36 | 11 | 11 |
| Mean | 306 | 20.4 | 23.6 | 46.5 | 27.1 | 32.4 | 2.16 |
| SD | 160 | 10.7 | 9.16 | 12.8 | 17.4 | 11.5 | 0.765 |
| Geometric Mean | 276 | 18.4 | 20.1 | 45.1 | 24.1 | 30.7 | 2.05 |
| Cycle 6 | | | | | | | |
| N | 6 | 6 | 6 | 0 | 6 | 0 | 0 |
| Mean | 302 | 20.1 | 22.1 | NC | 25.4 | NC | NC |
| SD | 90.2 | 6.01 | 7.28 | NC | 9.85 | NC | NC |
| Geometric Mean | 290 | 19.3 | 20.4 | NC | 23.8 | NC | NC |

Abbreviations: AUC = area under the curve; Clobs = observed systemic clearance; Cmax = maximum concentration; CPT = 20(S) camptothecin; HL = half-life; NC = not calculable; Tmax = time of maximum observed concentration; Vssobs = observed volume of distribution at steady state

Human plasma PK parameters were also measured in Study CRLX 001 for conjugated and unconjugated CPT after the first dose of CRLX-101 to the subject and after the first dose of the sixth monthly cycle (i.e., the 11th dose of CRLX-101). All subjects were dosed at 15 mg/m². No statistically significant differences are observed between the PK parameters from repeat dosing (see Table below), and no intra-subject changes are observed in AUC or clearance (FIG. 16; Human Plasma AUC and Clearance for Individual Subjects Measured after the First Dose and the Eleventh Dose).

TABLE
Average Human Plasma PK Parameters Measured After the First Dose and the
Eleventh Dose

| | | Conjugated CPT | | | | | Unconjugated CPT | |
|---|---|---|---|---|---|---|---|---|
| | | Cmax | T½ | AUCinf | CL | Vss | Cmax | Tmax |
| Dose # | N | (μg/L) | (hr) | (hr * mg/L) | (mL/hr) | (hr * mg/L) | (μg/L) | (hr) |
| Cycle 1 | 6 | 7333 ± | 27.6 ± | 270 ± | 105.7 ± | 2.8 ± | 363 ± | 24.9 ± |
| Day 1 | | 1777 | 2.0 | 53 | 19.1 | 0.5 | 292 | 0.2 |
| Cycle 6 | 6 | 7188 ± | 27.5 ± | 335 ± | 94 ± | 2.3 ± | 302 ± | 22.1± |
| Day 1 | | 1795 | 3.8 | 122 | 41 | 1.3 | 90 | 7.3 |

Abbreviations: AUC = area under the curve; Cl = systemic clearance; Cmax = maximum concentration; CPT = 20(S) camptothecin; T½ = half-life; Tmax = time of maximum observed concentration; Vss = volume of distribution at steady state.

Plasma exposure (AUC, left) and clearance (right) were measured for conjugated CPT for each subject after the first and eleventh dose and did not vary systematically. Lines connect individual subjects.

### Example 17: Drug Localisation and Pharmacodynamics in Gastric Tumour Tissue

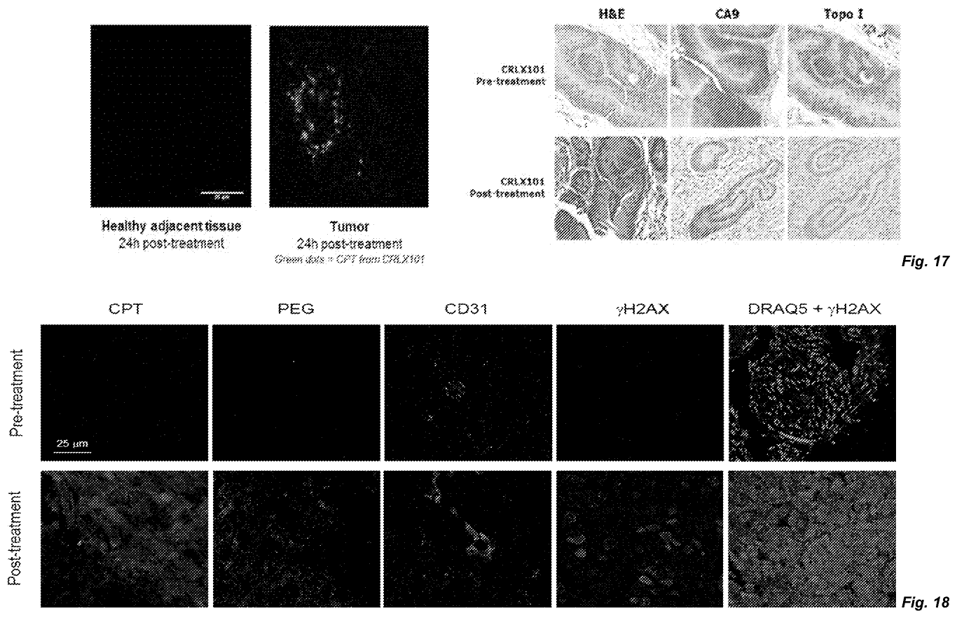

Drug localisation and pharmacodynamic effects of CRLX-101 were analysed in gastric tumour tissue obtained by subjects with advanced human epidermal growth factor receptor 2 (HER-2) negative gastric cancer in an IST performed at the City of Hope Comprehensive Cancer Center (NCT01612546). Tumour and adjacent non neoplastic tissues were obtained by endoscopic capture before and 24 to 48 hours after administration of the first dose of CRLX-101 monotherapy at 15 mg/m². Pre- and post-treatment biopsies were collected from a total of 10 subjects, 9 of whom had evaluable biopsies. The evaluable biopsies from all subjects were analysed for differential drug accumulation between tumour and non-neoplastic tissue using immunofluorescence techniques. CPT was visualised by direct fluorescent excitation of tissues. All 9 subjects showed clear evidence of CPT within the post-treatment tumour while only one subject showed potential evidence of CPT in the post treatment normal tissue, but that did not meet the requirements used to determine true CPT signals. An example of CPT fluorescence in post-treatment tumour and normal tissue is shown in FIG. 17 (CPT Localisation and Pharmacodynamic Effects in Gastric Cancer Tumours after First Administered Dose of CRLX-101; left). Tissue samples were also stained with an antibody against the PEG component of CRLX-101. In 5 of 9 subjects, the PEG antibody co-localised with the CPT fluorescence, suggesting intact nanoparticles were present within these post-treatment tumours. In 6 of the 9 gastric cancer subjects, enough post-treatment tumour tissue was obtained to also measure pharmacodynamic effects of CRLX-101 by immunohistochemistry, as shown in FIG. 17 (right). A haematoxylin and eosin stain was performed first to verify the quality of existing tumours and surrounding, uninvolved tissue. The CA9 antibody stain showed high intracellular expression in pre-treated tumour samples, whereas the posttreatment samples revealed much less staining, suggesting a decrease in HIF-1α, a transcription factor upstream of CA9. The topo-1 immunohistochemistry showed reduced staining from pretreatment to posttreatment samples. This result suggests CPT, released from the nanoparticle, bound Topo-I and triggered its degradation.

Camptothecin localises specifically in gastric tumour tissue and inhibits its intended targets, topoisomerase-1 and HIF-1α. Left, CPT fluorescence can be observed in tumour tissue but not adjacent non-neoplastic tissue 24 hours after administration of the first dose of CRLX-101, 15 mg/m². Scale bar is 20 μm. Right, Immunofluorescence of gastric tumour tissue shows inhibition of CA9 and topo-1 24 hours after administration of the first dose of CRLX-101, 15 mg/m².

### Example 18: Drug Localisation and Pharmacodynamics in Ovarian Tumour Tissue

Drug localisation and pharmacodynamic effects of CRLX-101 were also analysed in ovarian tumour tissue obtained from subjects with recurrent ovarian cancer in an IST (Group C) performed at Massachusetts General Hospital/Partners HealthCare (NCT01652079). Tumour tissues were obtained before and 6 days after administration of the first dose of CRLX-101 monotherapy at 15 mg/m². Pre- and post-treatment biopsies were collected from a total of 3 subjects, 2 of whom had biopsies of sufficient quality for analysis. The pre- and post-treatment biopsies were analysed for drug accumulation using immunofluorescence techniques. CPT was visualised by direct fluorescent excitation of tissues. Tissue samples were also stained with an antibody against the PEG component of CRLX-101. Tumour vasculature was stained with an antibody against cluster of differentiation 31 (CD31, also known as platelet endothelial cell adhesion molecule, or PECAM1). Double strand DNA breaks were visualised using an antibody against γH2AX. Nuclei were stained using the fluorescent stain DRAQ5™. As illustrated in FIG. 18 (CRLX-101 Localisation and Pharmacodynamic Effects in Ovarian Cancer Tumours after First Administered Dose of CRLX-101), post-treatment tissue showed evidence of that CRLX-101 and CPT are still present in tumour tissue 6 days after the first dose of CRLX-101. The staining of CPT and PEG was more diffuse than CD31, suggesting that CRLX-101 has penetrated tumours, away from the vasculature. The γH2AX staining suggests that DNA damage persists for at least 6 days after the first dose of CRLX-101.

CRLX-101 localises in ovarian tumour tissue and causes persistent DNA damage. CPT fluorescence and PEG can be observed in tumour tissue 6 days after administration of the first dose of CRLX-101, 15 mg/m². Staining of CPT and PEG are more diffuse than staining of CD31, suggesting that CRLX-101 has penetrated tumours away from the vasculature. γH2AX staining suggests that DNA double strand breaks persist for at least 6 days after administration of CRLX-101. Scale bar is 25 μm.

### Example 19: Phase 1 Clinical Study—CRLX-101 in Combination with Olaparib

A phase I study of CRLX-101 and olaparib was conducted in patients with advanced solid tumors.

Patients had histologically or cytologically documented unresectable, locally advanced, or metastatic solid tumors that were refractory to standard therapy and/or for whom no further standard therapy was available; Eastern Cooperative Oncology Group Performance Status of 0, 1 or 2; and adequate hematologic, renal and liver function.

CRLX-101 was administered as a 1-hour intravenous infusion on days 1 and 15 of a 28-day cycle. Normal saline (0.9% sodium chloride) was administered pre- and post-infusion of CRLX-101 (1 liter over 2 hours each). To reduce the risk of hypersensitivity reactions, patients received the following drugs 30-120 minutes prior to start of CRLX-101 infusion: a corticosteroid (dexamethasone 20 mg IV), an antihistamine (diphenhydramine 50 mg PO) and an H₂ antagonist (ranitidine 50 mg IV). Olaparib tablet at the appropriate dose level was administered twice daily by mouth on days 3-13 and 17-26. There was at least a 48-hour window between olaparib and CRLX-101.

A standard 3+3 design was used for dose escalation. Starting doses of CRLX-101 and olaparib tablets were 12 mg/m2 and 100 mg, approximately 80% and 33% of their respective single-agent MTDs. DLTs were based on toxicities observed during the first cycle. DLTs were defined as: Grade 4 neutropenia complicated by fever 38.5° C. (i.e. febrile neutropenia) and/or documented infection; grade 4 neutropenia or thrombocytopenia that does not resolve within 7 days; any grade 3-4 thrombocytopenia complicated with haemorrhage; grade 4 anaemia that does not resolve within 7 days despite optimal therapy (withholding study drug and red blood cell transfusions); inability to begin subsequent treatment course within 28 days of the scheduled date, due to study drug toxicity; any grade 3-4 non-haematologic toxicity (except fatigue/asthenia<2 weeks in duration; mucositis in subjects who have not received optimal therapy for mucositis; vomiting or diarrhoea lasting less than 72 hours whether treated with an optimal anti-emetic or antidiarrheal regimen or not; or alkaline phosphatase changes).

A new cycle of therapy did not begin until neutrophil count had recovered to >1500/mm³, platelets >75,000/mm³, haemoglobin 8 mg/dL and any toxicity recovered to ≤grade 2, with no more than a 3-week delay permitted. Study treatment was discontinued if there was a >3-week delay in reinstitution of treatment due to drug-related toxicity during a cycle. Dose reductions were allowed for toxicities. A maximum of two dose reductions were allowed. Dose re-escalation was not allowed.

This phase 1 clinical trial met its primary endpoint, identifying the maximum tolerated dose for CRLX-101 plus olaparib. Our findings show that the combination of CRLX-101 and olaparib is feasible and tolerable.

The most common toxicities were related to myelosuppression, which would likely have been further minimized if pegfilgrastim was administered prophylactically. There was only one instance of febrile neutropenia of grade 3 severity. Most common non-haematological toxicities were fatigue and nausea which were mild to moderate in all cases.

Although anti-tumor activity was not the main endpoint of the study, we recorded encouraging signals at the MTD/RP2D without additional toxicity.

Pharmacokinetic Analysis

Blood samples for pharmacokinetic (PK) analysis were drawn at predose CRLX-101, mid-infusion (30 min post start), end of infusion (EOI), and 1, 2, 12, 24, and 48 hr post EOI. Approximately 4 mL of blood was collected into a sodium heparin tube (BD Biosciences), immediately processed into plasma before storage in cryovials at −80 C. On C6D1, another set of blood samples for CRLX-101 measurement was collected to assess if any drug interactions exist and to assess any CRLX-101 accumulation. The total (bound+unbound to plasma proteins) plasma concentration of released (unconjugated) CPT from the CRLX-101 nanoparticle was measured by adding 0.1 normal hydrochloric acid to a small aliquot of plasma (to ensure all CPT in the lactone form), before further dilution and ultimate injection onto a Waters UPLC BEH RP18 Shield column (2.1×50 mm, 1.7 um; Waters Corp) for chromatographic separation prior to tandem mass spectrometric (MS/MS) detection on a AB Sciex QTRAP5500 (Sciex Corp, Foster City, Calif.). The total plasma concentration of all CPT present (unconjugated+conjugated to the nanoparticle) was measured by first adding 0.1 normal sodium hydroxide to induce base-catalyzed release of conjugated CPT from the nanoparticle (for 15 min) before addition of 0.1 N HCl to quench the reaction and force all fully-released CPT into the lactone form. From there, the procedure is the same as above. The calibration range was 1-10,000 ng/mL. Olaparib plasma concentrations were measured using a previously published assay (32), where the calibration range was 0.5-5,000 ng/mL.

First dose pharmacokinetic parameters were calculated using noncompartmental methods (Phoenix WinNonlin 8.0, Certara Pharsight Corp, Cary, N.C.). Any plasma concentration measured below the LLOQ was excluded from analyses. The maximum plasma concentration (CMAX) and time to CMAX (TMAX) were recorded as observed values. The area under the plasma concentration vs time curve to the last observed time point (AUCLAST) was calculated using the Linear Up Log Down trapezoidal rule. The elimination rate (kEL) was calculated as the slope of the log-transformed concentrations vs terminal time points. AUC extrapolated to time infinity (AUCINF) was calculated as AUCLAST+CLAST/kEL, where CLAST is the concentration at the last observed time point. Half-life (t½) was calculated as In2/kEL. Apparent oral clearance (CL/F) was calculated as dose/AUCINF; apparent oral volume of distribution (Vz/F) was calculated as CL/F divided by kEL.

Pharmacodynamic Analysis

PBMC and hair samples were obtained from all 24 patients on C1 D1 (pre-treatment), on C1D3 (48 hr after CRLX-101 and pre-olaparib) and on C1D4 (24 hr after olaparib). PBMCs were isolated by Ficoll gradient, washed 3 times with phosphate-buffered saline (PBS), fixed in 2% paraformaldehyde (PFA) for 20 min, washed 3 times with PBS, and spotted on slides by cytospin (800 rpm/4 min). PBMCs on slides were permeabilized with pre-chilled ethanol 70% and slides were stored at 4° C. overnight. PBMCs were then blocked for 30 min with 5% bovine serum albumin (BSA) in PBS-TT (PBS with 0.5% tween 20+0.1% triton X-100) before incubating 2 h with a mouse monoclonal anti-γ-H2AX antibody (Millipore, Billerica, Mass., USA) (dilution 500 in 1% BSA/PBS-TT) and then 1 h with a goat anti-mouse Alexa-488-conjugated IgG (Invitrogen, Eugene, Oreg., USA) (Dilution 500 in 1% BSA/PBS-TT). Finally, slides were incubated at 37° C. for 5 min with a solution containing RNAse A (0.5 mg/mL) and propidium iodide (PI) (5 μg/mL). Slides were then mounted with mounting medium containing PI (Vectashield, Vector Laboratories, Inc., Burlingame, Calif., USA) and sealed with nail polish.

Hair bulbs were collected in 1.5 microtubes filled with cold PBS and placed on ice directly after plucking. Plucked hairs were then fixed for 20 min at room temperature with 2% PFA in 1.5 microtubes. Following 3 washes with 1 ml PBS, plucked hairs were imaged and anagen hair bulbs hairs were selected for γ-H2AX detection. Samples were permeabilized with pre-chilled ethanol 70%, washed, and stored at 4° C. until use. γ-H2AX detection was performed as described for PBMCs but times for blocking and staining procedures were increased by 50%. Washes were performed by 3 washes with PBS. After incubation with the RNAse and PI solution at 37° C. for 30 min, hair shafts were detached from the bulbs by using a razor blade and hair bulbs were mounted between a slide and a coverslip with mounting medium containing PI and sealed with nail polish.

Samples were imaged by laser scanning confocal microscopy (Zeiss LSM 710 NLO). Optical sections through PBMCs and hair bulbs were combined in a maximum projection using the Zeiss Zen software. The foci were counted in PBMCs using the FociCounter software (33) by analyzing at least 200 cells, while γ-H2AX intensities were measured in the extremity of the hair bulbs with the Image J software.

Exome Sequencing

Whole exome sequencing was performed from macro-dissected, formalin-fixed paraffin-embedded tumors. Raw reads were trimmed using trimGalore (Martin, 2011) (v 0.6.5) and trimmomatic (v0.39). Surviving reads were aligned to the hg19 genome using the bwa mem (Li, 2013) aligner (v 0.7.17), followed by read sorting with samtools (Li et al., 2009) (v1.9).

Duplicate reads were marked using picard-tools (v 2.9.2, https://broadinstitute.github.io/picard/). Variants from tumor samples, and samples with matched normal and tumors were identified using mutect2 (Cibulskis et al., 2013) (v 4.1.0.0) and varscan2 (Koboldt et al., 2012) (v2.4.3), filtering for high quality variant. Identified variants were annotated with the dbSNP (Sherry et al., 2001) (v151) and clinvar (Landrum et al., 2018) (file date 2019-04-03) variants, followed by gene annotation with the snpEff (v4.3t) program. Copy-number alteration analysis and HRD score assignment for cases with matched tumor and normal pairs was performed with sequenza (Favero et al., 2015) (v3.0.0) and scarHRD (Sztupinszki et al., 2018) (v0.1.0) packages. We only included variants with frequency >95% in general population defined by National Heart, Lung, and Blood Institute's Trans-Omics for Precision Medicine Program, sequencing depth ≥20, variant allele fraction ≥10%, and not recurrently mutated in ≥3 patients in this cohort, were reported. For missense variants, we excluded variants annotated as “Benign” or “Likely benign” in ClinVar. Clinical benefit was defined as achieving partial response or stable disease lasting ≥3 months.

### Example 20: Urinary Excretion Data

Urine samples were collected from the subjects treated in Example 19 for determination of urinary excretion of polymer-conjugated and unconjugated CPT. For those subjects in the Phase 1 dose escalation cohort, urine samples were collected over 48 hours after dosing at intervals of 0-8, 8-24, and 24-48 hours or 0 24 and 24-48 hours after dosing. A spot urine PK sample was collected at 8, 24, 48, 168 (1 week) and 336 hours (pre-second dose) for Cycles 1 and 6 and immediately frozen for more accurate determination of total to unconjugated CPT ratio. A spot urine PK was also collected prior to dosing on Days 1 and 15 of every other cycle (Cycles 2, 3, 5, 7+). For those subjects in the Phase 2a MTD cohort, urine samples were collected for a 24-hr period prior to dosing on Days 1 and 15 of every cycle. A spot urine sample was also collected if the subject developed cystitis.

Urinary excretion across subjects was variable with a mean of 20.6% of the total administered dose of CRLX-101 excreted as CPT in the urine within the first 48 hrs. The majority of CPT excreted was in the polymer-conjugated form with a mean value of 16.2% of the total administered dose of CRLX-101 compared to an unconjugated mean value of 4.4% of the total administered dose of CRLX-101. There was considerable time dependency on urinary excretion of the polymer-conjugated CPT with the majority excreted in the first 24 hours, with a noteworthy decline in the 24- to 48-hr collection period. In addition, a subset of subjects who performed a 0- to 8-hr urine collection revealed the majority of the conjugated CPT was cleared within the first 8 hrs. Urinary clearance of unconjugated CPT was notably higher than that for polymer-conjugated CPT over all observation periods. Urinary clearance of unconjugated CPT in the first 48 hours post administration was similar over all collection periods and was not correlated with dose or creatinine clearance. Subjects dosed with more than one lot of CRLX-101 during their course of treatment did not show significant differences in the amount of CPT excreted.

The Tables below display conjugated and unconjugated urine clearance data following CRLX-101 infusion.

TABLE
CPT Conjugated and Unconjugated Urine Clearance-
0-24 hours and 24-48 hours

| | 0-24 h | 0-24 h | 24-48 h | 24-48 h | |
|---|---|---|---|---|---|
| | Conj. | Unconj. | Conj. | Unconj. | CrCl |
| Statistic | (L/hr) | (L/hr) | (L/hr) | (L/hr) | (L/hr) |
| Na | 8 | 8 | 8 | 8 | 8 |
| Mean | 0.0271 | 0.111 | 0.00338 | 0.120 | 5.38 |
| SD | 0.0134 | 0.0871 | 0.00431 | 0.0769 | 2.41 |
| Geometric Mean | 0.0235 | 0.0872 | NC | 0.0966 | 4.96 |

Abbreviations: CPT = 20(S) camptothecin;
CrCl = creatinine clearance. a Dose was 6 mg/m2 for 2 subjects, 12 mg/m2 for 3 subjects, and 18 mg/m2 for 3 subjects.

TABLE
CPT Conjugated and Unconjugated Urine Clearance - 0-8 hours, 8-24 hours,
and 24-48 hours

| | 0-8 | 0-8 | 8-24 h | 8-24 h | 24-48 | 24-48 | |
|---|---|---|---|---|---|---|---|
| | Conj. | Unconj. | Conj. | Unconj. | Conj. | Unconj. | CrCl |
| Statistic | (L/hr) | (L/hr) | (L/hr) | (L/hr) | (L/hr) | (L/hr) | (L/hr) |
| Na | 16 | 16 | 16 | 16 | 16 | 15 | 16 |
| Mean | 0.0692 | 0.238 | 0.0354 | 0.131 | 0.0265 | 0.238 | 4.74 |
| Standard | 0.0529 | 0.329 | 0.0432 | 0.0850 | 0.0233 | 0.137 | 1.41 |
| Deviation | | | | | | | |
| Geometric Mean | 0.0543 | 0.153 | 0.0210 | 0.104 | 0.0179 | 0.193 | 4.52 |

Abbreviations: CPT = 20(S) camptothecin; CrCl = creatinine clearance.
a Dose was 6 mg/m2 for 4 subjects, 12 mg/m2 for 3 subjects, 15 mg/m2 for 6 subjects, and 18 mg/m2 for 3 subjects.

TABLE
CPT Conjugated and Unconjugated Urine Clearance - 0-8 hours, 8-24 hours,
and 24-48 hours; MTD Evaluation at 15 mg/m²

| | | 0-8 | 8-24 h | 8-24 h | 24-48 | 24-48 | |
|---|---|---|---|---|---|---|---|
| | 0-8 Conj. | Unconj. | Conj. | Unconj. | Conj. | Unconj. | CrCl |
| Statistic | (L/hr) | (L/hr) | (L/hr) | (L/hr) | (L/hr) | (L/hr) | (L/hr) |
| N | 12 | 12 | 10 | 10 | 10 | 10 | 12 |
| Mean | 0.0299 | 0.0888 | 0.00742 | 0.0898 | 0.00623 | 0.101 | 4.65 |
| Standard | 0.0182 | 0.0716 | 0.00350 | 0.0545 | 0.00445 | 0.0603 | 1.36 |
| Deviation | | | | | | | |
| Geometric Mean | 0.0242 | 0.0571 | 0.00643 | 0.0732 | 0.00451 | 0.0857 | 4.46 |

Abbreviations: CPT = 20(S) camptothecin; CrCl = creatinine clearance.

In the Phase 2 study (CRLX 002), NSCLC subjects treated with CRLX-101 had urine collected for PK analysis to monitor and evaluate potential treatment-related cystitis adverse events (AEs). Pharmacokinetic analyses were completed and urine CPT concentrations results reviewed. No data trends were observed for subjects with AEs for cystitis or other urinary symptoms. Given the infrequent occurrence of ≤Grade 2 cystitis (9.3%), no >Grade 2 cystitis, and lack of notable CPT concentration levels, no further analyses were performed.

### Example 21: Phase 2 Clinical Study: CRLX-101 in Combination with Olaparib in Ovarian Cancer

It is anticipated that ˜60 patients (˜30 per cohort) will be enrolled into Phase 2A and ˜324 patients will be enrolled into Phase 2B; 132 in cohort 1 and 192 in cohort 2:

Cohort 1 patients (Phase 2A and 2B) must be/have:
PARP inhibitor naïve
Received no more than 1 prior line of therapy which must be platinum-based chemotherapy AND have either:
Stable disease (SD) following treatment with first line platinum-based chemotherapy as defined by RECIST v1.1 criteria OR
Primary platinum resistant disease defined by progressive disease (PD) within ≥1 and ≤6 months after completion of first line platinum-based chemotherapy as defined by RECIST v1.1 criteria.

Cohort 2 patients (Phase 2A and 2B) must have:
Received at least 2 prior lines of treatment, 1 of which must be platinum-based chemotherapy
Received a PARP inhibitor in the maintenance setting as their most recent treatment following a confirmed response by RECIST1.1 (CR or PR) to the last regimen which must be a platinum-based chemotherapy, with maintenance of response by PARP inhibitor lasting ≥6 and up to 12 months, with subsequent confirmed disease progression as defined by RECIST v1.1 criteria.

Therefore, in total it is estimated that ˜384 patients will be enrolled into the study.

CRLX-101 will be supplied as a solution for iv administration, which will be presented in glass vials.

CRLX-101 will be supplied as a lyophilized cake for reconstitution with sterile water for injection (SWFI) in a 30-mL single-use amber glass vial. Each vial contains 35 mg of CPT equivalents (approximately 350 mg of polymer drug conjugate). The formulation contains mannitol at a w/w ratio of 1:1.25 CRLX-101:mannitol. The drug appears as a white to slightly yellow solid and a clear, colorless to slightly yellow solution upon reconstitution with water.

CRLX-101 is manufactured by University of Iowa Pharmaceuticals, US, and packaged, stored and distributed by Almac, UK. Drug will be supplied in cartons, each containing a single amber glass vial containing 35 mg drug product. Vials must be stored refrigerated, between 2-8° C., until ready for use. As with all cytotoxic drugs, take care when handling and preparing CRLX-101.

CRLX-101 will be administered at a dose of 12 mg/m2 on Days 1 and 15 of a 28 day cycle.

Instructions will be provided in the clinical study protocol on how patients will receive pretreatment with steroids/antihistamine due to hypersensitivity reactions to CRLX-101 seen in previous trials.

Instructions will be provided in the clinical study protocol on preparing patients for dosing to minimize bladder/urinary tract toxicity as per IB.

Olaparib (film coated tablets) bd oral 250 mg on days 3-12 and 17-26; Morning dose only on days 13 and 27 allowing a 48 hr window between CRLX-101 and olaparib dosing.

Commercial supplies of olaparib will be provided packaged in original blister strips, presented in study treatment wallets. The 250 mg dose will be made up of 1×100 mg film coated tablet (containing 100 mg olaparib and 0.24 mg sodium per tablet) plus 1×150 mg film coated tablet (containing 150 mg olaparib and 0.35 mg sodium per tablet). The 100 mg tablet is a yellow, oval, bi-convex tablet, with OP100 on one side, whilst the 150 mg tablet is a green/grey oval bi-convex tablet with OP 150 on one side.

Dose and treatment schedules are described in the Table below.

TABLE
Dose and Treatment Schedule

| Investigational | Pharmaceutical | | |
|---|---|---|---|
| drug | Form | Dose | Frequency and/or Regimen |
| CRLX-101 | Lyophilised cake for | 12 mg/m2 | IV on Days 1 and 15 of |
| | reconstitution with | | each 28-day cycle |
| | WFI | | |
| Olaparib | 1 × 100 mg film | 250 mg BID | Oral, BID on days 3-12 and 17-26; |
| | coated tablet plus 1 × | | Morning dose only on days 13 and 27, |
| | 150 mg film coated | | allowing a 48-hr window between |
| | tablet | | CRLX-101 and olaparib dosing |

Patients enrolled into this study will receive treatment with CRLX-101 and olaparib, which will be dosed in cycles, each consisting of 28 days. CRLX-101 will be administered as a single IV dose on Days 1 and 15 of each 28 day Cycle. Olaparib will be administered BID on days 3-12 and 17-26 with a morning dose only on days 13 and 27 to allow a 48 hr window between CRLX-101 and olaparib dosing. A dosing window of 48 hr+/−4 hr is acceptable.

Duration of Treatment

Patients receiving CRLX-101 in combination with olaparib can continue the combination treatment until confirmed disease progression, provided they have not met any other discontinuation criteria and the Investigator believes it is in the patient's best interest.

Patient in the SOC arm can receive up to a maximum of 6 cycles of chemotherapy.

Phase 2A

It is anticipated that ˜60 patients (˜30 patients per cohort) will be enrolled into Phase 2A. Both treatment cohorts will open in parallel and patients may be enrolled into each cohort concurrently.

The efficacy data will be assessed for futility after approximately 15 patients have been enrolled in each cohort (futility may be assessed independently for each of the two cohorts) and suitable for efficacy assessment.

In the event of toxicities, the dose of olaparib and/or CRLX-101 may be modified and a revised dose and/or schedule selected for Phase 2B. Dose modification guidelines will be provided in the clinical study protocol.

The data will be formally summarised at the end of Phase 2A. Informal summaries will be conducted during the enrolment of patients into Phase 2A of the study, to confirm safety of the combinations and to explore for early signs of efficacy. Based on these descriptive analyses during and/or upon the completion of cohorts 1 and 2, a decision may be made to initiate Phase 2B of the study.

Primary Endpoints
ORR as measured using RECIST1.1
Safety and tolerability; AEs, SAEs, vital signs, haematology and clinical chemistry, ECGs, dose interruptions and reductions, withdrawals due to treatment related toxicities, duration of treatment

Secondary Endpoints
PFS
OS
PFS by BRCA (germline and somatic) and HRD status
OS by BRCA (germline and somatic) and HRD status
Duration of response
Duration of response by BRCA (germline and somatic) and HRD status
ORR by BRCA (germline and somatic) and HRD status
Pharmacokinetics

Phase 2B

Phase 2B will also be comprised of two treatment cohorts.

It is anticipated that ˜324 patients will be enrolled into this part of the study, ˜132 in Cohort 1 and ˜192 in Cohort 2. Both treatment cohorts will open in parallel and patients may be enrolled into each cohort concurrently.

In the event of toxicities, the dose of olaparib, the SOC chemotherapy and/or CRLX-101 may be modified. Dose modification guidelines will be provided in the clinical study protocol.

An Independent Data Monitoring Committee (IDMC) will be set-up to conduct periodic reviews of safety data in Phase 2B. The IDMC will meet on a quarterly basis and as required. Based on the periodic or ad hoc safety data reviews the IDMC can make a recommendation to continue, amend, or stop the study at any point for safety reasons.

The following endpoints for patients treated with CRLX-101 plus olaparib will be compared with those for patients treated with SoC for both cohorts 1 and 2.

Primary Endpoint
PFS

Secondary Endpoints
OS
Safety and tolerability; AEs, SAEs, vital signs, haematology and clinical chemistry, ECGs, dose interruptions and reductions, withdrawals due to treatment related toxicities, duration of treatment
OS by BRCA (germline and somatic) and HRD status
PFS by BRCA (germline and somatic) and HRD status
ORR
ORR by BRCA (germline and somatic) and HRD status
Duration of response
Duration of response by BRCA (germline and somatic) and HRD status
QoL EORTC Quality-of-Life Questionnaire (QLQ-C30) and the ovarian cancer module (QLQ-OV28) and EQ-5D

Efficacy Assessments

RECIST 1.1 guidelines for measurable, non-measurable, target lesions (TLs) and non-target lesions (NTLs) and the objective tumour response criteria are to be followed.

Baseline CT/MRI should be performed of the chest, abdomen and pelvis with additional anatomy based on signs and symptoms of individual patients. Baseline assessments should be performed no more than 28 days before the start of study, and ideally should be performed as close as possible to the start of study treatment. The methods of assessment used at baseline should be used at each subsequent follow-up assessment. Follow-up assessments should be performed every 8 weeks (±7 days) after the start of study drug for 52 weeks, and thereafter every 12 weeks (±2 weeks) until disease progression.

Any other sites at which new disease is suspected should also be appropriately imaged. If an unscheduled assessment is performed and the patient has not progressed, every attempt should be made to perform subsequent assessments at the scheduled visits whilst the patient remains on study drug. However, if an unscheduled scan has been conducted within 2 weeks of the scheduled scan, it is not necessary to scan again at the scheduled time point, unless clinically indicated. The patient can be scanned at the subsequent scheduled timepoint.

Categorisation of objective tumour response assessment will be based on the RECIST 1.1 guidelines for response: CR (complete response), PR (partial response), SD (stable disease) and PD (progression of disease).

If the Investigator is in doubt as to whether progression has occurred, particularly with response to NTLs or the appearance of a new lesion, it is advisable to continue treatment and reassess the patient's status at the next scheduled assessment or sooner if clinically indicated.

To achieve ‘unequivocal progression’ on the basis of non-target disease, there must be an overall level of worsening in non-target disease such that, even in presence of SD or PR in target disease, the overall tumour burden has increased sufficiently to merit discontinuation of treatment. A modest ‘increase’ in the size of one or more NTLs is usually not sufficient to qualify for unequivocal disease progression status.

### Example 22: Efficacy Study of CRLX-101 Alone and in Combination with Olaparib in a Range of Cell Lines In Vitro

### SUMMARY

| | Efficacy study of CRLX-101 alone and in |
|---|---|
| | combination with Olaparib in a range of cell |
| Study title | lines in vitro. |
| Test | Olaparib |
| substance ID | CRLX-101 |
| Formulation | Olaparib-DMSO |
| | CRLX-101-Water for injection |
| Dosing range | Olaparib-30, 10, 3, 1, 0.3 μM |
| | CRLX-101-100, 30, 10, 3, 1, 0.3, 0.1, 0.03 nM |
| Incubation Time | 72 hours |
| Summary of | Viability of 9 cell lines (DU145, Capan-1, Hs 766T, |
| observations | PANC-1, NUGC4, HEY, OVCAR3, OAW28 and NCI- |
| | H510A) will be determined following incubation with |
| | Olaparib in combination with CRLX-101 using an |
| | MTS assay. After 72 hour incubation with compound, |
| | cell titre aqueous will be added and absorbance |
| | quantified at 490 nm using a plate-reader. |
| | IC50 values for inhibition of cell growth will be |
| | calculated using GraphPad Prism software by |
| | nonlinear regression, with bottom and top |
| | constraints at 0 and 100%, respectively. |

1.0 Assay Protocol

MTS Assay

Day 1: Plate Cells

The optimal seeding density for each cell line (DU145, Capan-1, Hs 766T, PANC-1, NUGC4, HEY, OVCAR3, OAW28 and NCI-H510A) will be determined prior to running the assay. Cells will be seeded in 50 μL media (in triplicate for each condition). Three 96 well flat bottom tissue culture plates will be required per cell line (See appendix I). Cells will be allowed to adhere overnight at 37° C., 5% CO₂, 20% O₂ before compound is added.

Day 2: Treat Cells
Prepare a stock solution for each compound as follows
LI Olaparib—30 mM in DMSO
LI CRLX-101-100 μM in water
Dilute each compound to 4×the required largest concentration in complete media (1:250 dilution).
LI Olaparib—120 μM
LI CRLX-101-400 nM
Make a series of 4 half log dilutions for Olaparib and 7 half log dilutions for CRLX-101 from the highest concentration in complete media (containing 0.4% DMSO or 0.4% water).
Add 25 μL of each diluted compound to the relevant wells as indicated in the “Plate plans for MTS assay” table below (untreated wells will receive 50 μL of complete media, vehicle wells will receive 50 μL of complete media containing 0.4% DMSO and monotherapy well will also receive 25 μL of complete media containing 0.4% DMSO/0.4% water). This will yield the final desired concentration in each well.
Incubate plates for 72 hours at 37° C., 5% CO₂, 20% 02.

Day 5: Analysis of Proliferation
Remove plates from the incubator and perform a Cell Titre 96 Aqueous One Solution Cell Proliferation Assay (Promega #G3582) according to the manufacturers protocol.
Record absorbance at 490 nm using colorimetric plate reader.

Data Analysis

The percentage inhibition will be calculated against the mean of the DMSO treated control samples and the IC50 values for inhibition of cell growth will be calculated using GraphPad Prism software by nonlinear regression with bottom and top constraints at 0 and 100%, respectively.

Plate Plans for MTS Assay

Plate 1,2,3

| | | | | | | | | | | | |
|---|---|---|---|---|---|---|---|---|---|---|---|
| media | media | media | media | media | media | media | media | media | media | media | media |
| media | 0 μM, | 0 μM, | 0 μM, | 0 μM, | 0 μM, 1 nM | 0 μM, 3 nM | 0 μM, | 0 μM, 30 nM | 0 μM, 100 nM | untreated | media |
| | 0 nM | 0.03 nM | 0.1 nM | 0.3 nM | | | 10 nM | | | | |
| media | 0.3 μM, | 0.3 HM, | 0.3 μM, | 0.3 HM, | 0.3 μM, 1 nM | 0.3 μM, 3 nM | 0.3 μM, | 0.3 μM, 30 nM | 0.3 HM, 100 nM | untreated | media |
| | 0 nM | 0.03 nM | 0.1 nM | 0.3 nM | | | 10 nM | | | | |
| media | 1 μM, | 1 μM, | 1 μM, | 1 μM, | 1 μM, 1 nM | 1 μM, 3 nM | 1 μM, | 1 μM, 30 nM | 1 μM, 100 nM | vehicle | media |
| | 0 nM | 0.03 nM | 0.1 nM | 0.3 nM | | | 10 nM | | | | |
| media | 3 μM, | 3 μM, | 3 μM, | 3 μM, | 3 μM, 1 nM | 3 μM, 3 nM | 3 μM, | 3 μM, 30 nM | 3 μM, 100 nM | vehicle | media |
| | 0 nM | 0.03 nM | 0.1 nM | 0.3 nM | | | 10 nM | | | | |
| media | 10 μM, | 10 μM, | 10 μM, | 10 μM, | 10 μM, 1 nM | 10 μM, 3 nM | 10 μM, | 10 μM, 30 nM | 10 μM, 100 nM | media no | media |
| | 0 nM | v nM | 0.1 nM | 0.3 nM | | | 10 nM | | | cells | |
| media | 30 μM, | 30 μM, | 30 μM, | 30 μM, | 30 μM, 1 nM | 30 μM, 3 nM | 30 μM, | 30 μM, 30 nM | 30 μM, 100 nM | media no | media |
| | 0 nM | 0.03 nM | 0.1 nM | 0.3 nM | | | 10 nM | | | cells | |
| media | media | media | media | media | media | media | media | media | media | media | media |

0, 0 = Olaparib, CRLX-101

Results

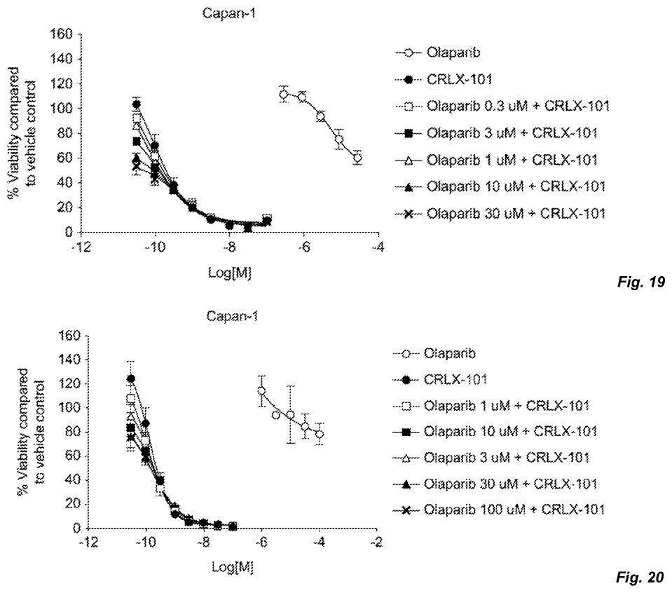

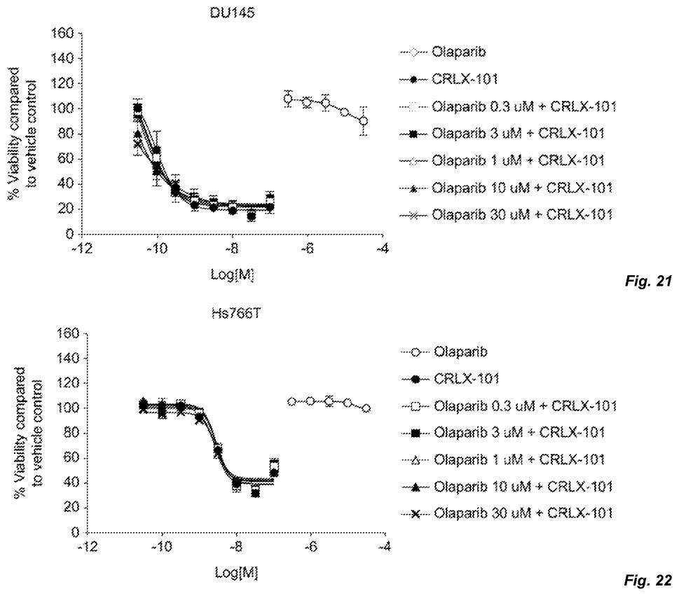

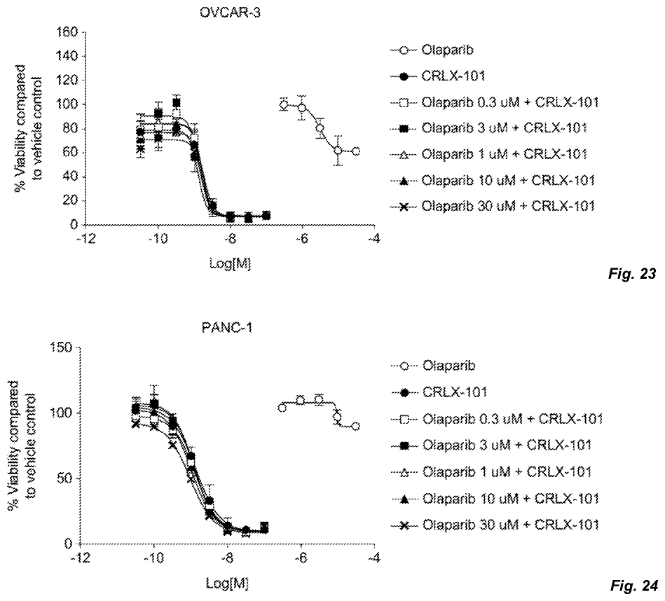

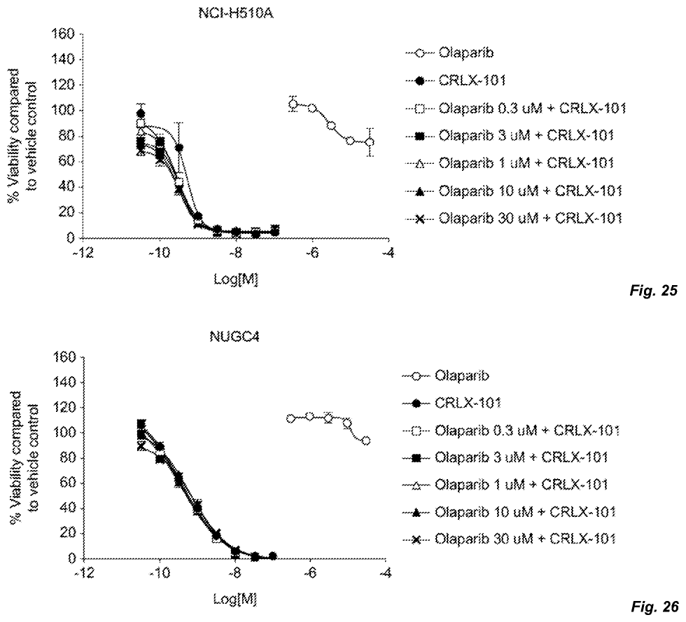

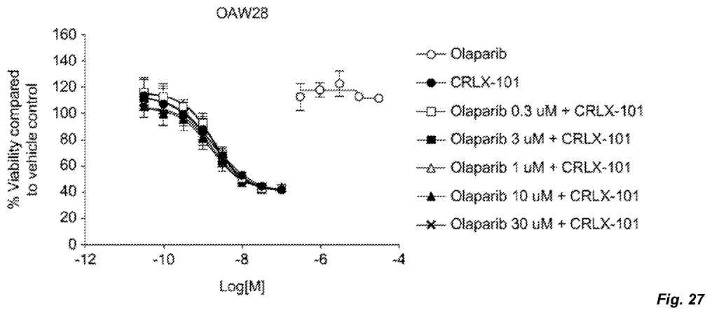

The in vivo study (Example 22) was performed by Axis Bio Ltd, on behalf of Ellipses Pharma. The results for each cell line are reported in FIGS. 19 to 28 (Capan-1 schedule 1—FIG. 19; Capan-1 (schedule 2)—FIG. 20; DU145—FIG. 21; Hs 766T—FIG. 22; OVCAR3—FIG. 23; PANC-1—FIG. 24; NCI-H510A—FIG. 25; NUGC4—FIG. 26; and OAW28—FIG. 27), showing % viability compared to vehicle controls. The HEY study will be performed in the future.

IC50 (relative and absolute) are as follows:

Capan-1 (schedule 1)

| Compound | IC50 relative | IC50 Absolute |
|---|---|---|
| Olaparib | 5.713 μM | 40.05 μM |
| CRLX-101 | 0.0874 nM | 0.2506 nM |
| Olaparib 0.3 μM + CRLX-101 | 0.0609 nM | 0.2113 nM |
| Olaparib 1 μM + CRLX-101 | 0.0498 nM | 0.1738 nM |
| Olaparib 3 μM + CRLX-101 | 0.1197 nM | 0.1271 nM |
| Olaparib 10 μM + CRLX-101 | 0.2330 nM | 0.0755 nM |
| Olaparib 30 μM + CRLX-101 | 0.4347 nM | 0.0523 nM |

Capan-1 (Schedule 2)

In modified schedule 2 (modified as compared to schedule 1 above, which was in accordance with the testing methodology outlined above), CRLX-101 was added on the first day, then on the second day all media was removed and fresh CRLX-101 and Olaparib were added together for the remaining testing period.

| Compound | IC50 relative | IC50 Absolute |
|---|---|---|
| Olaparib | 0.00546 μM | 487.7 μM |
| CRLX-101 | 0.1407 nM | 0.2713 nM |
| Olaparib 1 μM + CRLX-101 | 0.1135 nM | 0.2227 nM |
| Olaparib 3 μM + CRLX-101 | 0.1733 nM | 0.2075 nM |
| Olaparib 10 μM + CRLX-101 | 0.1857 nM | 0.1806 nM |
| Olaparib 30 μM + CRLX-101 | 0.1680 nM | 0.1488 nM |
| Olaparib 100 μM + CRLX-101 | 0.1753 nM | 0.1440 nM |

DU145

| | | |
|---|---|---|
| Olaparib | 11.62 μM | 93.12 μM |
| CRLX-101 | 0.0945 nM | 0.2921 nM |
| Olaparib 0.3 μM + CRLX-101 | 0.0606 nM | 0.3047 nM |
| Olaparib 1 μM + CRLX-101 | 0.0480 nM | 0.3117 nM |
| Olaparib 3 μM + CRLX-101 | 0.0200 nM | 0.3038 nM |
| Olaparib 10 μM + CRLX-101 | 0.0081 nM | 0.1866 nM |
| Olaparib 30 μM + CRLX-101 | 0.0277 nM | 0.1580 nM |

Hs 766T

| | | |
|---|---|---|
| Olaparib | 14.08 μM | Not calculated |
| CRLX-101 | 2.6439 nM | 15.07 nM |
| Olaparib 0.3 M + CRLX-101 | 2.739 nM | 18.39 nM |
| Olaparib 1 μM + CRLX-101 | 2.733 nM | 21.74 nM |
| Olaparib 3 μM + CRLX-101 | 2.824 nM | 19.79 nM |
| Olaparib 10 μM + CRLX-101 | 2.921 nM | 18.28 nM |
| Olaparib 30 μM + CRLX-101 | 2.754 nM | 14.06 nM |

OVCAR3

| | | |
|---|---|---|
| Olaparib | 3.029 μM | 43.4 μ.M |
| CRLX-101 | 1.767 nM | 1.008 nM |
| Olaparib 0.3 μM + CRLX-101 | 1.590 nM | 1.484 nM |
| Olaparib 1 μM + CRLX-101 | 1.594 nM | 1.457 nM |
| Olaparib 3 μM + CRLX-101 | 1.394 nM | 1.448 nM |
| Olaparib 10 μM + CRLX-101 | 1.253 nM | 0.9278 nM |
| Olaparib 30 μM + CRLX-101 | 1.577 nM | 0.6638 nM |

PANC-1

| | | |
|---|---|---|
| Olaparib | 9.258 μM | 79.18 μM |
| CRLX-101 | 1.348 nM | 1.942 nM |
| Olaparib 0.3 μM + CRLX-101 | 1.313 nM | 1.607 nM |
| Olaparib 1 μM + CRLX-101 | 1.202 nM | 1.750 nM |
| Olaparib 3 μM + CRLX-101 | 1.245 nM | 1.776 nM |
| Olaparib 10 μM + CRLX-101 | 1.034 nM | 1.396 nM |
| Olaparib 30 μM + CRLX-101 | 0.9379 nM | 0.9726 nM |

NCI-H510A

| | | |
|---|---|---|
| Olaparib | 2.661 μM | Not calculated |
| CRLX-101 | 0.5165 nM | 0.4238 nM |
| Olaparib 0.3 μM + CRLX-101 | 0.2642 nM | 0.2494 nM |
| Olaparib 1 μM + CRLX-101 | 0.3066 nM | 0.2293 nM |
| Olaparib 3 μM + CRLX-101 | 0.3286 nM | 0.1816 nM |
| Olaparib 10 μM + CRLX-101 | 0.3011 nM | 0.1589 nM |
| Olaparib 30 μM + CRLX-101 | 0.3275 nM | 0.1329 nM |

NUGC4

| | | |
|---|---|---|
| Olaparib | ~11.47 μM | not calculated |
| CRLX-101 | 0.3668 nM | 0.6484 nM |
| Olaparib 0.3 μM + CRLX-101 | 0.3624 nM | 0.6013 nM |
| Olaparib 1 μM + CRLX-101 | 0.3277 nM | 0.5778 nM |
| Olaparib 3 μM + CRLX-101 | 0.3731 nM | 0.5557 nM |
| Olaparib 10 μM + CRLX-101 | 0.6361 nM | 0.6956 nM |
| Olaparib 30 μM + CRLX-101 | 0.8076 nM | 0.6146 nM |

OAW28

| | | |
|---|---|---|
| Olaparib | ~8.877 μM | Not calculated |
| CRLX-101 | 1.84 nM | 22.93 nM |
| Olaparib 0.3 μM + CRLX-101 | 1.965 nM | 22.57 nM |
| Olaparib 1 μM + CRLX-101 | 1.864 nM | 21.18 nM |
| Olaparib 3 μM + CRLX-101 | 1.779 nM | 21.4 nM |
| Olaparib 10 μM + CRLX-101 | 1.616 nM | 19.01 nM |
| Olaparib 30 μM + CRLX-101 | 1.775 nM | 20.04 nM |

### Example 23: Efficacy Study of CRLX-101 Alone and in Combination with Olaparib in Various Xenograft Models

Summary

| | Efficacy study of CRLX-101 alone and in |
|---|---|
| | combination with Olaparib in various xenograft |
| Study title | models. |
| Animal species | Mouse |
| Animal strain | Balb/c nude and NOD SCID nude |
| Animal gender | SKOV3 - female; Panc-1 - female; NCI-N87 - male; |
| | PC3 - male; 22Rv1 - male |
| Tumour cell line | SKOV3 |
| | Panc-1 |
| | NCI-N87 |
| | PC3 |
| | 22Rv1 |
| Route of tumour | Subcutaneous |
| implantation | |
| Test substance ID | CRLX-101 |
| | Olaparib |
| Dose | CRLX-101 5 mg/kg |
| | Olaparib 100 mg/kg |
| Route of admin. | CRLX-101 - IV |
| | Olaparib - PO |
| Formulation | CRLX-101 - Sterile Water for Injection (WFI) |
| | Olaparib - 10% DMSO: 30% HP-B-CD in water |
| Duration of dosing | CRLX-101 - Single IV dose (Day 1) |
| | Olaparib - QD orally on Days 2-14 |
| Dosing volume | CRLX-101 - 5 ml/kg |
| | Olaparib - 10 ml/kg |
| Summary of study | Tumour volume three times weekly (Mon/Wed/Fri) |
| | Bodyweight three times weekly (Mon/Wed/Fri) |
| | General signs/symptoms |
| | Tumours resected whole, divided into two sections |
| | one snap frozen and other formalin fixed |

1.0 Animal Management

1.1 Animals: A total of 84 male Balb/c nude mice and 56 female Balb/c nude mice aged 5-8 weeks weighing approximately 25-30 g will be used for the study. These will be purchased from Charles River.

1.2 Animal acclimatization: Seven days.

1.3 Allocation to treatment groups: Allocation to treatment groups will be done randomly. Random allocation of animals to treatment groups will be carried out using Graphpad—see link www.graphpad.com/quickcalcs/randomize1.cfm

1.4 Animal Housing: Mice will be housed in IVC cages (5 mice per cage) with individual mice identified by tail marking. Cages, bedding and water will be sanitized before use. Animals will be provided with suitable bedding to provide environment enrichment and nesting material. All investigators entering the animal holding room will wear appropriate barrier clothing (e.g. Tyvex suits, gloves, appropriate footwear and mask). Each cage will be clearly labeled with a card indicating the number of animals, sex, strain, DOB, study number, licence number, start date and treatment. All animals will have free access to a standard certified commercial diet and water. Cages will be changed once a week with food and water replaced when necessary.

The animal holding room will be maintained as follows—room temperature at 18-24° C., humidity at 55-70% and a 12 h light/dark cycle used.

1.5 Aseptic Technique: All preparation of dosing solutions, tumour measurements, and dosing will be carried out in a sterile biosafety cabinet.

1.6 Ethics: All protocols to be used in this study have been approved by the Axis Bio Animal Welfare and Ethical Review Committee, and all procedures are carried out under the guidelines of the Animal (Scientific Procedures) Act 1986.

2.0 Test Substance and Formulation

| Name | CRLX-101 |
|---|---|
| Batch no. | 04310156 |
| Storage conditions | 2-8° C. protected from light |
| Name | Olaparib |
| Batch no. | 42 |
| Storage conditions | 2-8° C. protected from light |

2.1 CRLX-101 formulation instructions

Dosing solution will be prepared fresh at the beginning of the study. Any dosing solution remaining at the end of the study will be discarded. Dosing volume will be 5 ml/kg for IV dosing.

CRLX-101 Formulation

Preparation of a 5 mg/kg CRLX-101 dosing solution in sterile WFI (35 mg vial preparation example)

Allow the vial of CRLX-101 to equilibriate to room temperature for approx. 1 h. Add 14 ml of sterile WFI to the 35 mg vial of CRLX-101 using a sterile syringe to generate a 2.5 mg/ml stock solution. Insert the needle through the vial septum and slowly add the sterile WFI along the inside of the vial wall to minimise foaming. Dissolve the product by gently swirling (do not shake) until a clear homogenous solution is formed. If required an orbital shaker may be used for up to 30 minutes to dissolve the product (do not vortex). The solution should be inspected every 2-3 minutes. Further dilute the product in sterile WFI to a 1 mg/ml working solution. Product is stable for 24 h at room temperature after reconstitution.

The dosing solution will be protected from light and mixed well before administration.

2.2 Olaparib formulation instructions

Dosing solution will be prepared fresh every seven days. Any dosing solution remaining at the end of the seven days will be discarded. Dosing volume will be 10 ml/kg for PO dosing.

Weigh required amount of compound in appropriate size vessel (label this as vessel A). Example: To prepare 10 ml of 100 mg/kg dose (i.e. 10 mg/ml), weigh out of 100 mg of olaparib into an appropriate vessel. Add 10% v/v DMSO into vessel A and mix to dissolve the contents (either using vortex mixer or sonication). Example: add 1 ml DMSO. In a separate appropriate size vessel (label this as vessel B) transfer 50% v/v of final volume of 60% w/v HP-13-CD in sterile deionized water and an appropriate size magnetic flea. Stir the contents continuously on magnetic stirrer (make sure a vortex is created throughout). Example: Add 5 ml of 60% HP-13-CD to vessel B. Slowly (drop wise) transfer the contents from vessel A into vessel B, while continuously stirring. Closely watch the contents in vessel B if precipitation is observed wait for the precipitates to dissolve and then continue slowly transferring these contents into vessel B. Repeat this process if any precipitation is observed again. (Note: don't rinse the vessel A with water and transfer into vessel B). Leave the above solution stirring for approximately 30 mins to ensure all particles (if any) have dissolved, visually check to ensure clear solution has formed.

Transfer the contents from vessel B into an appropriate size volumetric flask, rinse vessel B with water and transfer into volumetric flask and make up to mark with sterile deionized water, add water slowly. Example: Add 4 ml of water. This will yield a 30% w/v HP-13-CD final concentration. Mix the solution by inverting.

The olaparib solution should be stored at room temperature and checked visually on a daily basis. If visual checks detect evidence of precipitation or particle accumulation on the bottom of the vial the formulation should be disposed of and a fresh formulation prepared. Protect from light required during storage, wrap clear glass bottle/vial in foil. Do not store in amber bottle as this will impair ability to see possible particle accumulation on bottom of the vial. The dosing solution is stable for 7 days at room temperature (protected from light) in the range of concentrations used for this study. One vial is made for 7 days but if any precipitation is observed, for the further preparations the total volume of 7 days will be divided into 2 vials (1 vial for the 4 first days and 1 vial for the last 3 days).

3.0 Study Information

3.1 Study Purpose

The purpose of this study is:
To determine the efficacy of CRLX-101 alone and in combination with Olaparib in the SKO3 ovarian cancer in NOD SCID nude mice, and Panc-1 pancreatic cancer, NCI-N87 gastric cancer, PC3 prostatic cancer and 22Rv1 prostatic cancer xenograft models in Balb/c nude mice.

4.0 Study Design

Tumour Cell Implantation:

Panc-1, NCI-N87, PC3 and 22Rv1 cells (1×10⁷ cells per animal; 1:1 with matrigel) will be implanted subcutaneously on the right flank of Balb/c nude mice using a 22G needle. The same approach was followed for SKOV3, but with NOD SCID nude mice. When tumours reach approx. 200 mm³ animals will be randomly assigned to the following treatment groups:

| Group | n | Treatment | Dose | Dosing route | Dosing regimen |
|---|---|---|---|---|---|
| 1 | 6 | Vehicle | n/a | IV | Single dosing |
| | | | | | occasion |
| | | | | | (Day 1) |
| | | | | PO | QD (days 2-14) |
| 2 | 6 | CRLX-101 | 5 mg/kg | IV | Single dosing |
| | | | | | occasion |
| | | | | | (Day 1) |
| 3 | 6 | Olaparib | 100 mg/kg | PO | QD (days 2-14) |
| 4 | 6 | CRLX-101 + | 5 mg/kg + | IV | Single dosing |
| | | Olaparib | 100 mg/kg | | occasion |
| | | | | | (Day 1) |
| | | | | PO | QD (days 2-14) |

Duration of dosing: Single IV dose on day 1 and/or QD PO dosing from day 2 to day 14.

Observations to continue out to day 30-45

Individual dose: Calculated from bodyweight recorded on day of dosing.

Serial Observations

Tumour Measurement

Tumours will be measured three times per week using digital calipers (Mon/Wed/Fri). The length, width and depth of the tumour will be measured and used to calculate the tumour volume. Tumour volume will be entered onto an excel spreadsheet which will be sent to the client on a regular basis.

Bodyweight

The bodyweight of all mice on the study will be measured and recorded daily; this information will be used to calculate precise dosing for each animal. The Sponsor will be informed if any animal loses more than 10% bodyweight.

General Signs and Symptoms

Mice will be observed daily and any signs of distress or changes to general condition e.g. starred fur, lack of movement, difficulty breathing will be noted.

5.0 Sampling

Tumour

On the final day of the study animals will be euthanised via CO₂ inhalation. Tumours will be resected whole, weighed and divided into two portions. One portion will be snap frozen and stored at −80° C. The other portion will be formalin fixed for 48 h at room temperature before being transferred to 70% EtOH at room temperature.

All samples will be returned to the client at the close of the study at the address above.

Results

The in vitro study (Example 23) was performed at Axis Bio Ltd, on behalf of Ellipses Pharma.

In a variation to the protocol above, the SKOV3 study instead used 20 female NOD SCID nude mice (i.e. 5 per group) instead of Balb/c nude mice.

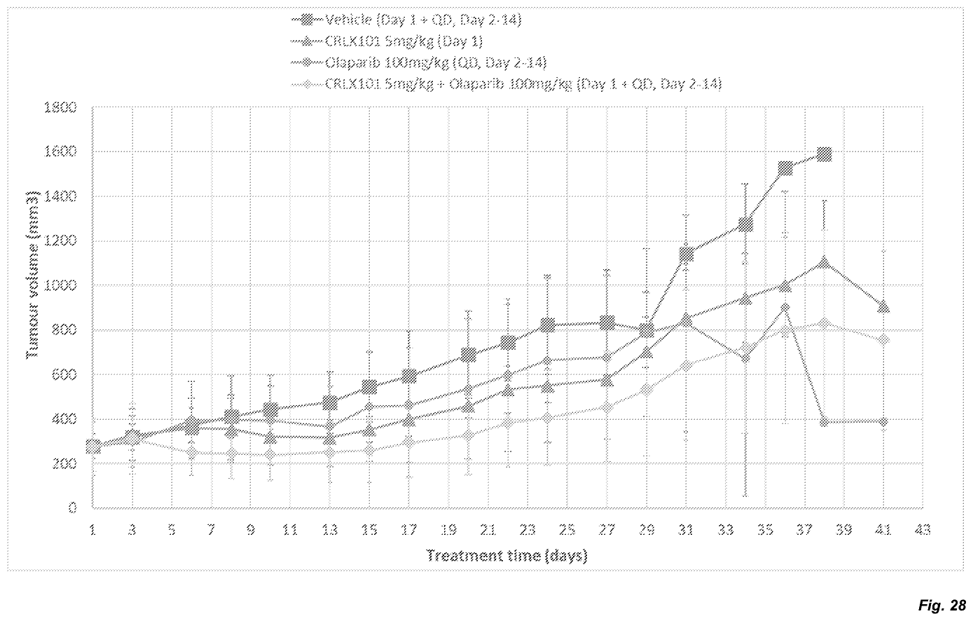

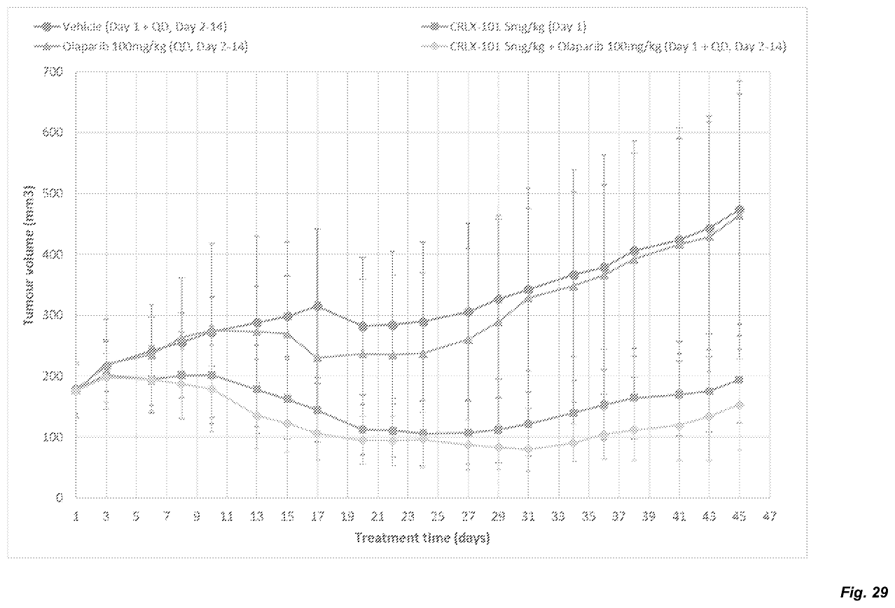

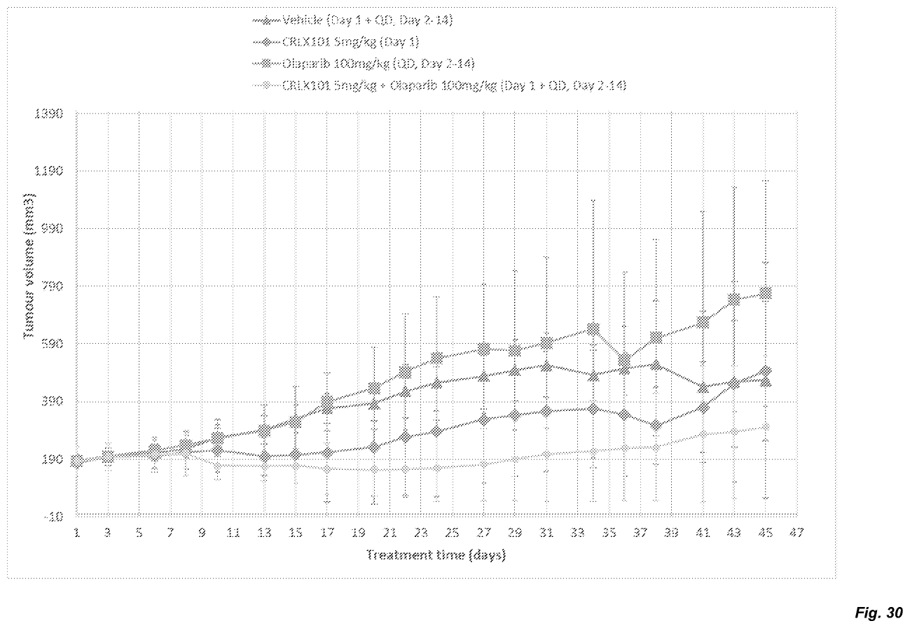

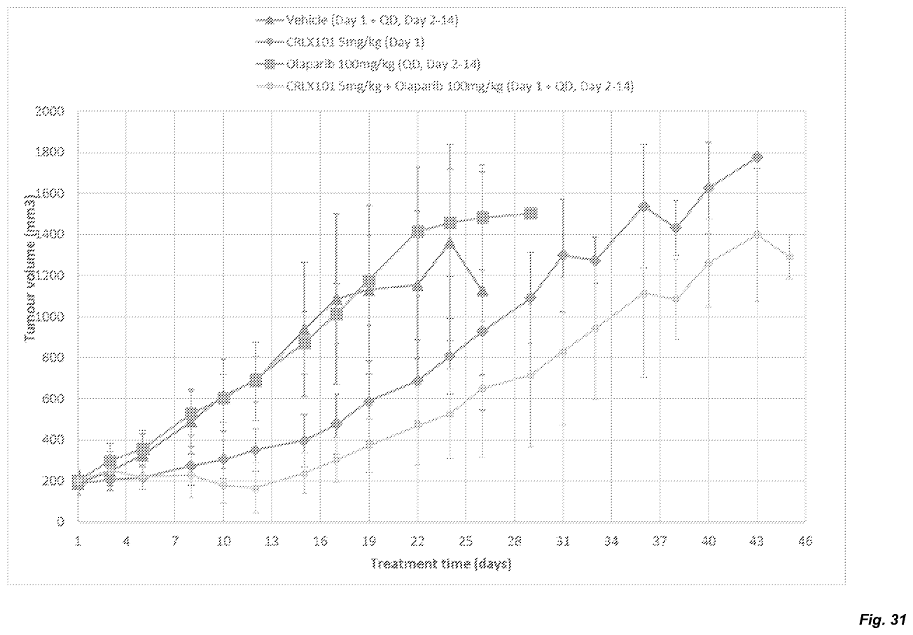

The results for each cell line are reported in FIGS. 28 to 31 (SKOV3—FIG. 28; NCI-N87—FIG. 29; PC— FIG. 30; 22Rv1—FIG. 31), showing % viability compared to vehicle controls. The Panc-1 study will be performed in the future.

### Example 24: Phase 2 Clinical Study: CRLX-101 in Combination with Olaparib in Ovarian Cancer (Updated Protocol)

It is anticipated that up to approximately 60 patients ('30 patients per cohort) will be enrolled into Phase 2A. Both cohorts will open in parallel and patients will be enrolled into each cohort concurrently.

The efficacy data will be assessed for futility after approximately 15 patients have been enrolled in each cohort (futility will be assessed independently for each of the 2 cohorts).

In the event of toxicities, the dose of Olaparib and/or CRLX-101 may be modified and a revised dose and/or schedule selected for Phase 2B.

Phase 2B

Phase 2B will be randomised and will be comprised of 2 cohorts:
Cohort 1 will explore CRLX-101 in combination with Olaparib compared with SOC chemotherapy (TBC) in patients with advanced platinum resistant ovarian cancer who are PARP inhibitor naive and who have received no more than 1 prior line of therapy which must be platinum-based chemotherapy (n='132). Randomisation to treatment arms will be stratified by 1) Response to first line platinum therapy, i.e. SD versus progression ≥1 and ≤6 months, after first line platinum therapy and 2) BRCA/HRD status
Cohort 2 will explore CRLX-101 in combination with Olaparib compared with SOC chemotherapy (TBC) in patients with advanced ovarian cancer who have received at least 1 prior line of therapy which must include at least 1 line of platinum-based chemotherapy followed by a PARP inhibitor as maintenance treatment as their last therapy (n=˜192). Randomisation to treatment arms will be stratified by 1) BRCA/HRD status 2) dose of PARP inhibitors (i.e. those on standard dose and those with dose reductions and 3) number of prior lines of therapy 1 or >1 4) platinum free interval, 6-12 months or >12 months It is anticipated that approximately 324 patients will be enrolled into Phase 2B. Both cohorts may open in parallel and patients may be enrolled into each cohort concurrently.

In the event of toxicities, the dose of Olaparib, the SOC chemotherapy and/or CRLX-101 may be modified.

Patient Populations

Cohort 1: Patients with advanced platinum resistant ovarian cancer (see inclusion criteria for definition of platinum resistant) who are PARP inhibitor naive and who have received no more than 1 prior line of therapy which must be platinum-based chemotherapy.

Cohort 2: Patients with advanced ovarian cancer who are platinum resistant/partially platinum sensitive and have progressed following at least 1 prior line of therapy which must include at least 1 line of platinum-based chemotherapy followed by a PARP inhibitor as maintenance treatment as their last therapy.

Safety Review Committee

A Safety Review Committee (SRC) will meet on a quarterly basis and as required to review the safety and PK data from patients enrolled into Phase 2A. Any dose interruptions or reductions of CRLX-101 or Olaparib will be taken into consideration.

The SRC will make an assessment for futility after approximately 15 patients have been enrolled into a cohort, based on the efficacy data and emerging risk:benefit profile. At the end of Phase 2A, the SRC will guide the decision to initiate 1 or both cohorts in Phase 2B, or terminate further recruitment into the study.

Definition of Study Periods

The study is made up of 4 study periods: (1) Screening, (2) Treatment, (3) Safety Follow Up, and where appropriate, (4) Long Term Follow Up: PFS and OS.

Treatment Duration

Patients can continue receiving CRLX-101 in combination with Olaparib until confirmed disease progression, provided they have not met any other discontinuation criteria and the Investigator believes it is in the patient's best interest.

Follow Up

Patients are at any time free to withdraw from the study (study drug and assessments), without prejudice to further treatment (withdrawal of consent). Such patients will always be asked about the reason(s) and the presence of any AEs. If possible, they will be seen by the Investigator and undergo the assessments and procedures scheduled for the post-study assessment. Safety assessment will continue until 30 days following completion of study drug.

If treatment is discontinued for reasons other than disease progression or complete withdrawal of consent, the patient should be encouraged to continue tumour assessments until disease progression or death.

STUDY OBJECTIVES AND ENDPOINTS

| Phase 2A Primary Objectives | Phase 2A Endpoints for Primary Objectives |
|---|---|
| Cohort 1 | Cohorts 1 and 2 |
| To investigate the efficacy of CRLX-101 in combination with Olaparib in | Overall Response Rate as measured using RECIST1.1 |
| patients with advanced platinum resistant ovarian cancer (see inclusion | Safety and tolerability; AEs, SAEs, dose interruptions and reductions, |
| criteria for definition of 'platinum resistant') who are PARP inhibitor | withdrawals due to treatment related toxicities, duration of treatment, |
| naive and who have received no more than 1 prior line of therapy | vital signs, haematology, clinical chemistry and ECGs. |
| which must be platinum-based chemotherapy | |
| Cohort 2 | |
| To investigate the efficacy of CRLX-101 in combination with Olaparib in | |
| patients with advanced ovarian cancer who have received at least 1 | |
| prior line of therapy which must include at least 1 line of platinum- | |
| based chemotherapy followed by PARP inhibitor maintenance as their | |
| most recent treatment and have progressive disease whilst receiving | |
| PARP inhibitor maintenance treatment confirmed by RECIST1.1 | |
| Cohorts 1 and 2 | |
| To explore the safety and tolerability of CRLX-101 when combined with | |
| Olaparib in the indicated patient populations | |
| Phase 2A Secondary Objectives | Phase 2A Endpoints for Secondary Objectives |
| Cohort 1 | Cohorts 1 and 2 |
| To further investigate the efficacy of CRLX-101 in combination with | PFS |
| Olaparib in patients with advanced platinum resistant ovarian cancer | PFS by BRCA (germline and somatic) and HRD status |
| (see inclusion criteria for definition of 'platinum resistant') who are | OS |
| PARP inhibitor naive and who have received no more than 1 prior line | OS by BRCA (germline and somatic) and HRD status |
| of therapy which must be platinum-based chemotherapy | Duration of response |
| Cohort 2 | Duration of response by BRCA (germline and somatic) and HRD |
| To further investigate the efficacy of CRLX-101 in combination with | status |
| Olaparib in patients with advanced ovarian cancer who have received | ORR by BRCA (germline and somatic) and HRD status |
| at least 1 prior line of therapy which must include at least 1 line of | Pharmacokinetics parameters: |
| platinum-based chemotherapy followed by PARP inhibitor maintenance | Maximum plasma concentration (Cmax), time to Cmax (tmax), terminal |
| as their most recent treatment and have progressive disease whilst | plasma half-life (t½λz), area under the plasma concentration-time |
| receiving PARP inhibitor maintenance treatment confirmed by | curve from zero to infinity (AUCSS), oral plasma clearance (CL/F), oral |
| RECIST1.1 | volume of distribution during terminal phase (Vz/F), mean residence |
| Cohorts 1 and 2 | time (MRT). |
| Pharmacokinetics | |
| Phase 2B Primary Objectives | Phase 2B Endpoints for Primary Objectives |
| Cohort 1 | Cohorts 1 and 2 |
| To investigate the efficacy of CRLX-101 in combination with Olaparib | Progression Free Survival |
| vs SoC chemotherapy (TBC) in patients with advanced platinum | |
| resistant ovarian cancer (see inclusion criteria for definition of platinum | |
| resistant) who are PARP inhibitor naive and who have received no | |
| more than 1 prior line of therapy which must be platinum-based | |
| chemotherapy. | |
| Cohort 2 | |
| To investigate the efficacy of CRLX-101 in combination with Olaparib | |
| vs SoC chemotherapy (TBC) in patients with advanced ovarian cancer | |
| who have received at least 1 prior line of therapy which must include at | |
| least 1 line of platinum-based chemotherapy followed by PARP | |
| inhibitor maintenance as their most recent treatment and have | |
| progressive disease whilst receiving PARP inhibitor maintenance | |
| treatment confirmed by RECIST1.1 | |
| Phase 2B Secondary Objectives | Phase 2B Endpoints for Secondary Objectives |
| Cohort 1 | The following endpoints for patients treated with CRLX-101 plus |
| To further investigate the efficacy of CRLX-101 in combination with | Olaparib will be compared with those for patients treated with SoC for |
| Olaparib vs SoC chemotherapy in | both Cohorts 1 and 2 |
| patients with advanced platinum resistant ovarian cancer (see inclusion | OS |
| criteria for definition of platinum resistant) who are PARP inhibitor | Safety and tolerability; AEs, SAEs, dose interruptions and reductions, |
| naive and who have received no more than 1 prior line of therapy | withdrawals due to treatment related toxicities, duration of treatment, |
| which must be platinum-based chemotherapy | vital signs, haematology, clinical chemistry and ECGs |
| Cohort 2 | OS by BRCA (germline and somatic) and HRD status |
| To further investigate the efficacy of CRLX-101 in combination with | PFS by BRCA (germline and somatic) and HRD status |
| Olaparib vs SOC chemotherapy in patients with advanced ovarian | ORR |
| cancer who have received at least 1 prior line of therapy which must | ORR by BRCA (germline and somatic) and HRD status |
| include at least 1 line of platinum-based chemotherapy followed by | Duration of response |
| PARP inhibitor maintenance as their most recent treatment and have | Duration of response by BRCA (germline and somatic) and HRD |
| progressive disease whilst receiving PARP inhibitor maintenance | status |
| treatment confirmed by RECIST1.1 | QOL EORTC Quality-of-Life Questionnaire (QLQ-C30) and the |
| Cohorts 1 and 2 | ovarian cancer module (QLQ-OV28) and EQ-5D |
| To explore the safety of CRLX-101 when combined with Olaparib in the | |
| indicated patient populations versus SOC chemotherapy | |
| Patient Quality of Life (QoL) | |
| Exploratory Objectives | Endpoints for Exploratory Objectives |
| Exploratory analyses may or may not be performed during the course | Serum CA 125 |
| of the study. Where available at time of publication, results from | To analyse biomarkers predictive of the response to CRLX-101 and |
| exploratory analyses will be reported in the Clinical Study Report | Olaparib |
| (CSR). Where testing is performed after the study has completed, | |
| these results will be appended to the CSR. | |

Population Selection Criteria

Patients must meet all the following Inclusion Criteria to be eligible for inclusion in the study:
1. Patients aged 18 years of age at the time of informed consent
2. Ability to understand and provide written informed consent prior to undergoing any study procedures
3. Life expectancy of >3 months, as estimated by the investigator
4. Histologically confirmed diagnosis (cytology alone excluded) with high-grade serous ovarian cancer or high-grade endometrioid ovarian cancer, including primary peritoneal or fallopian tube cancer
5. BRCA mutational status is known (germline and somatic). (For Patients in Phase 2A, status does not need to be known prior to enrolment)
6. HRD status is known. (For Patients in Phase 2A, status does not need to be known prior to enrolment)
7. At least 1 measurable lesion to assess response by RECIST v1.1 criteria
8. Archival tumour sample must be available. In the absence of an archival tumour biopsy, a tumour tissue biopsy will need to be collected prior to enrolment
9. Eastern Cooperative Oncology Group (ECOG) Performance Status of 0 or 1 at Screening
10. Normal organ and bone marrow function:
   Haemoglobin ≥9.0 g/dL
   Absolute neutrophil count (ANC)≥1.5×10⁹
   Lymphocyte count≥0.5×10⁹
   Platelet count≥100×10⁹
   Total bilirubin≤1.5 institutional upper limit normal (ULN)
   Serum albumin≥2.5 g/dL
   AST and ALT≤2.5×ULN, unless liver metastases are present in which case they must be ≤5×ULN
   Serum creatinine ≤1.5×ULN or calculated creatinine clearance >50 mL/min (calculated using the Cockroft-Gault formula) for patients with creatinine levels above institutional normal
   Patients not receiving anti-coagulant medication must have an INR of ≤1.5 and an aPTT≤1.5×ULN
11. In the opinion of the Investigator, all other relevant medical conditions must be well-managed and stable for at least 28 days prior to first administration of study drug
12. Willing and able to participate in all required evaluations and procedures in this study protocol
13. Contraception: Each female subject of childbearing potential must agree to use a highly effective method of contraception (i.e., a method with less than 1% failure rate per year [e.g., sterilization, hormone implants, hormone injections, some intrauterine devices, vasectomized partner, or combined birth control pills]) from screening until 6 months after the last dose of CRLX-101 or Olaparib, whichever was taken last. Females of childbearing potential must have a negative serum pregnancy test at Screening and a negative serum or urine pregnancy test within 24 hours prior to CRLX-101 dosing on Day 1 of each Cycle (and must not be lactating). Each female subject will be considered to be of childbearing potential unless she has been surgically sterilised by hysterectomy or bilateral tubal ligation/salpingectomy or has been postmenopausal for at least 1 year.

Cohort 1 Patients (Phase 2A and 2B) Must be/have:
14. PARP inhibitor naïve
15. Received no more than 1 prior line of therapy which must be platinum-based chemotherapy
16. Either: Stable disease (SD) following treatment with first line platinum based chemotherapy OR Primary platinum resistant disease defined by progressive disease (PD) within ≥1 and ≤6 months after completion of first line platinum-based chemotherapy

Cohort 2 Patients (Phase 2A and 2B) Must have:
17. Received at least 1 prior lines of treatment, 1 of which must be platinum-based chemotherapy
18. Received a PARP inhibitor in the maintenance setting as their most recent treatment following a confirmed response by RECIST1.1 (CR or PR) to the last regimen which must be a platinum-based chemotherapy, with maintenance of response by PARP inhibitor lasting ≥6 months, with subsequent confirmed disease progression whilst receiving PARP inhibitor maintenance treatment as defined by RECIST v1.1 criteria

Exclusion Criteria

Patients with any of the following will not be included in the study:
1. Non-epithelial tumour of the ovary, the fallopian tube or the peritoneum
2. Ovarian tumours of low malignant potential or low grade
3. Prior treatment with a topoisomerase I inhibitor
4. Potent inhibitors or inducers of CYP3A4
5. Concurrent treatment with Coumadin (Warfarin)
6. History of stroke, transient ischemic attack, or myocardial infarction, within 6 months prior to C1D1
7. Brain and/or leptomeningeal metastases that are symptomatic or untreated or that require current therapy. Brain imaging must not be older than 12 weeks (at the start of screening). Results with abnormal/unexpected findings of brain MRI should be discussed with the Medical Monitor as part of the screening process
8. Systemic anti-cancer therapy for the disease under study within 3 weeks or 5 half-lives, whichever is longer, of the first dose of study drug
9. Ongoing toxic manifestations of previous treatments. Exceptions to this are alopecia or certain Grade 2 toxicities, which in the opinion of the Investigator should not exclude the patient.
10. Patients considered by the Investigator to be at a higher baseline risk for new onset cystitis (see Section 7.4.4)
11. Patients with a history, or features suggestive, of bone marrow dysplasia or myelodysplastic syndrome (MDS) or acute myeloid leukaemia (AML)
12. Confirmed QTcF>470 msec on screening ECG or congenital long QT syndrome
13. Receiving an investigational anti-cancer treatment concurrently or within 3 weeks or 5 half-lives of either the parent drug or any active metabolite, whichever is longer, prior to the first dose of study drug
14. Any evidence of severe or uncontrolled systemic conditions (e.g., severe hepatic impairment) or current unstable or uncompensated respiratory or cardiac conditions which makes it undesirable for the patient to participate in the study or which could jeopardize compliance with the protocol
15. Hypersensitivity to CRLX-101 or any of its excipients
16. Known history of Human Immunodeficiency Virus infection (HIV) (testing is not required), active infection with SARS-CoV-2, hepatitis B virus (HBV) or hepatitis C virus (HCV) per institutional protocol. Testing for HBV or HCV status is not necessary unless clinically indicated or the patient has a history of HBV or HCV infection. All patients should be tested for an active SARS-CoV-2 infection with an approved diagnostic test kit
17. Malignant disease other than that being treated in this study, with the following exceptions:
   Malignancies that were treated curatively and have not recurred within 2 years prior to study treatment
   Completely resected basal cell and squamous cell skin cancers
   Any malignancy considered to be indolent and that has never required therapy
   Completely resected carcinoma in situ of any type
18. Any medical condition that would, in the investigator's judgment, prevent the patient's participation in the clinical study due to safety concerns, compliance with clinical study procedures, or interpretation of study results
19. Any major surgical procedure (in the investigator's judgement) within 2 weeks of the first dose of study drug
20. Pregnant, likely to become pregnant, or lactating women (where pregnancy is defined as the state of a female after conception and until the termination of gestation)
21. Palliative radiotherapy (e.g., for pain or bleeding) within 6 weeks prior to randomisation or patients who have not completely recovered (Grade ≥2) from the effects of previous radiotherapy
22. Uncontrolled pleural effusion, pericardial effusion, or ascites requiring recurrent drainage procedures (once monthly or more frequently) Note: Patients with indwelling catheters (e.g., PleurX) are allowed
23. Hypersensitivity or intolerance (due to safety or other reasons) to PARP inhibitors

Cohort 1 Patients (Phase 2A and 2B) Who:
24. Have primary platinum refractory disease defined as progression during first line treatment with 4-6 cycles of platinum based chemotherapy

Cohort 2 Patients (Phase 2A and 2B) Who:
25. Progress within 6 months during PARP inhibitor maintenance treatment
26. Progress after completion of PARP inhibitor maintenance treatment
CRLX-101 will be supplied as a lyophilized cake for reconstitution with sterile water for injection (SWFI) in a 30-mL single-use amber glass vial. Each vial contains 35 mg of CPT equivalents (approximately 350 mg of polymer drug conjugate). The formulation contains mannitol at a w/w ratio of 1:1.25 CRLX-101:mannitol. The drug appears as a white to slightly yellow solid and a clear, colorless to slightly yellow solution upon reconstitution with water.
Commercial supplies of Olaparib will be provided packaged in original blister strips, presented in study treatment wallets. The 250 mg dose will be made up of 1×100 mg film coated tablet (containing 100 mg Olaparib and 0.24 mg sodium per tablet) plus 1×150 mg film coated tablet (containing 150 mg Olaparib and 0.35 mg sodium per tablet). The 100 mg tablet is a yellow, oval, bi-convex tablet, with OP100 on one side, whilst the 150 mg tablet is a green/grey oval bi-convex tablet with OP 150 on one side.

Dose and treatment schedules are described in the Table below.

| Study | Pharmaceutical | | Frequency and/or |
|---|---|---|---|
| drug | Form | Dose | Regimen |
| CRLX- | Lyophilised | 12 mg/m² | IV on Days 1 and 15 |
| 101 | cake for | | of each 28-day cycle |
| | reconstitution | | |
| | with WFI | | |
| Olaparib | 1 × 100 mg film | 250 mg | Oral, BID on days |
| | coated tablet | BID | 3-12 and 17-26; |
| | plus 1 × | | Morning dose only |
| | 150 mg film | | on days 13 and 27, |
| | coated | | allowing a 48-hr |
| | tablet | | window between |
| | | | CRLX-101 and |
| | | | Olaparib dosing |

Patients enrolled into this study will receive treatment with CRLX-101 and Olaparib, which will be dosed in cycles, each consisting of 28 days. CRLX-101 will be administered as a single IV dose on Days 1 and 15 of each 28-day cycle. Olaparib will be administered BID on days 3-12 and 17-26 with a morning dose only on days 13 and 27 to allow a 48 hr window between CRLX-101 and Olaparib dosing. A dosing window of 48 hr+/−4 hr is acceptable.

CRLX-101 is manufactured by University of Iowa Pharmaceuticals, US, and packaged, stored and distributed by Almac, UK. Drug will be supplied in cartons, each containing a single amber glass vial containing 35 mg drug product. Vials must be stored refrigerated, between 2-8° C., until ready for use. As with all cytotoxic drugs, take care when handling and preparing CRLX-101.

Commercial supplies of Olaparib (Lynparza®) will be purchased, and packaged in study wallets by Almac, UK. Each wallet will contain supplies for either days 3-13 or days 17-27. Prepared CRLX-101 will be infused intravenously over 60 minutes on Days 1 and 15 of each cycle. Nothing else should be added to the bag. The CRLX-101 infusion should begin as soon as possible following preparation, and diluted CRLX-101 infusion solution not used within 6 hours should be destroyed following institutional practices.

Olaparib tablets should be swallowed whole, with or without food. Olaparib tablets will be taken by the patient at home on the morning and evening on Days 3-12 and 17-26 and a single dose on the morning of Days 13 and 27, of each cycle. Each dose will be made up of 1×100 mg film coated tablet plus 1×150 mg tablet, total dose 250 mg.

Efficacy Assessments

RECIST 1.1 guidelines (Eisenhauera 2009) for measurable, non-measurable, target lesions (TLs) and non-target lesions (NTLs) and the objective tumour response criteria are to be followed.

Baseline CT/MRI should be performed of the chest, abdomen and pelvis with additional anatomy based on signs and symptoms of individual patients. Baseline assessments should be performed no more than 28 days before the start of study, and ideally should be performed as close as possible to the start of study treatment. The methods of assessment used at baseline should be used at each subsequent follow-up assessment. Follow-up assessments should be performed every 8 weeks (−7 days) after the start of study drug for 52 weeks, and thereafter every 12 weeks (−2 weeks) until disease progression per RECIST 1.1.

Should a patient stop study treatment for reasons other than progressive disease, they will continue to be followed for PFS, with assessments being performed every 12 weeks (±2 weeks).

Any other sites at which new disease is suspected should also be appropriately imaged. If an unscheduled assessment is performed and the patient has not progressed, every attempt should be made to perform subsequent assessments at the scheduled visits whilst the patient remains on study drug. However, if an unscheduled scan has been conducted within 2 weeks of the scheduled scan, it is not necessary to scan again at the scheduled time point, unless clinically indicated. The patient can be scanned at the subsequent scheduled timepoint.

If a patient discontinues study treatment for reasons other than progressive disease or withdrawal of consent, a CT/MRI scan should be performed at the EOT visit, provided the patient has not had a CT/MRI scan within the previous 6 weeks.

Categorisation of objective tumour response assessment will be based on the RECIST 1.1 guidelines for response: CR (complete response), PR (partial response), SD (stable disease) and PD (progression of disease).

For Phase 2A, responses (CR or PR) will require confirmation. A confirmed response of CR/PR means that a response of CR/PR is recorded at 1 visit and confirmed by repeat imaging at least 4 weeks later with no evidence of progression between confirmation visits. There is no formal requirement per RECIST v1.1 for confirmation of response in the randomised Phase 2B and therefore the next scan may be performed as scheduled.

If the Investigator is in doubt as to whether progression has occurred, particularly with response to NTLs or the appearance of a new lesion, it is advisable to continue treatment and reassess the patient's status at the next scheduled assessment or sooner if clinically indicated.

To achieve ‘unequivocal progression’ on the basis of non-target disease, there must be an overall level of substantial worsening in non-target disease such that, even in presence of SD or PR in target disease, the overall tumour burden has increased sufficiently to merit discontinuation of treatment. A modest ‘increase’ in the size of 1 or more NTLs is usually not sufficient to qualify for unequivocal disease progression status.

Safety and Tolerability Assessments

Safety will be monitored by assessing physical examination, vital signs, ECG, weight, performance status, haematology, chemistry, urinalysis, as well collecting AEs at every visit. AEs will be graded according to the Common Terminology Criteria for Adverse Events (CTCAE) version 5.

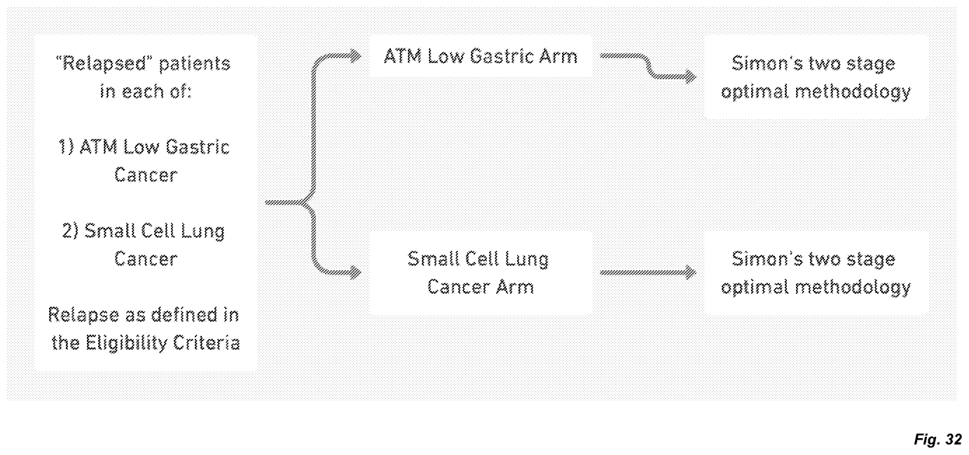

### Example 25: A Phase 2 Multi-Arm Open Label Study of CRLX-101 in Combination with Olaparib in Defined Populations of Relapsed Advanced/Metastatic Gastric and Small Cell Lung Cancer Tumours

| | |
|---|---|
| Study Number | CRLX-101-202 |
| Sponsor | Ellipses Pharma, Ltd. |
| Investigational | The term Investigational Agent in this study refers to CRLX-101. The |
| Agent | Investigational Agent (CRLX-101) is a nanoparticle-drug conjugate |
| | containing a payload of camptothecin administered intravenously. Study |
| | drug will be supplied by the Sponsor, Ellipses Pharma Ltd. |
| Study Type | Multi-arm, multi-centre open label phase 2 study with two Simon two |
| | stage arms |
| Study Purpose | Topoisomerase \| inhibitors are established chemotherapy agents that |
| and Rationale | have utility across a arrange of tumour types. These agents work by |
| | interrupting DNA replication through generation of single strand breaks in |
| | DNA that ultimately result in irreversible DNA replication defects and |
| | subsequent cell cycle arrest and cell death. |
| | The Investigational Agent (CRLX-101) is a nanoparticle-drug conjugate |
| | containing a payload of camptothecin, a topisomerase I inhibitor, that is |
| | administered intravenously. Nanoparticle drug conjugates are designed |
| | to selectively accumulate in tumour tissue via leaky tumour vasculature |
| | and accumulate in tumour cells via micropinocytosis, thus selectively |
| | targeting tumour cells, whilst reducing the toxicity of the conjugated active |
| | drug to normal tissues. For CRLX-101 the nanoparticles are designed to |
| | result in slow release of the active drug at the site of the tumour to |
| | maximise tumoural exposure to camptothecin and maximise tumour cell |
| | killing. CRLX-101 has shown to have antitumour activity across a range |
| | of solid tumours in preclinical models and preliminary evidence for activity |
| | of CRLX-101 has been observed in a Phase 1/2 trial in solid tumours with |
| | an acceptable tolerability profile. |
| | Poly (ADP-ribose) polymerases (PARP) 1 and 2 are enzymes activated |
| | by DNA damage which facilitate DNA repair in pathways involving single- |
| | strand breaks (SSBs) and base excision repair (BER). DNA replication |
| | and error-repair is a critical component of cancer cell survival. PARP |
| | inhibition leads to stalling of replication forks due to accumulation of |
| | unrepaired SSBs. Stalled replication forks degrade into highly cytotoxic |
| | double strand breaks (DSBs) if not corrected by appropriate repair |
| | mechanisms. BRCA mutated (BRCAm) and Homologous Repair |
| | Deficient (HRD) tumours are particularly sensitive to PARP inhibition as |
| | they cannot correct DSBs, resulting in genomic instability and cell death. |
| | Targeting PARP is now an established treatment strategy in a range of |
| | tumours, including pancreatic, breast cancer and ovarian cancer with a |
| | number of PARP inhibitors approved to treat BRCA mutated, HRD |
| | tumours or all comer patient populations. |
| | There has been substantial interest in the potential to combine |
| | topoisomerase inhibitors with PARP inhibitors due to the potentially |
| | synergistic mechanisms of action with regard to DNA strand breakage |
| | and inhibition of DNA repair. It is thought that inhibition of DNA repair may |
| | enhance the activity of Topisomerase inhibitors where these are used |
| | currently, and conversely addition of topisomerase inhibitors to PARP |
| | inhibitor may enhance the activity of PARP inhibitors in sensitive tumours. |
| | However, numerous attempts to combine standard topoisomerase |
| | inhibitors with PARP inhibitors have been unsuccessful due to over- |
| | lapping toxicity. |
| | Early clinical data for the combination of CRLX-101 and olaparib has |
| | demonstrated that the two can be combined at clinically relevant doses |
| | with acceptable toxicity. Preclinical studies have demonstrated that the |
| | combination is synergistic in terms of providing greater efficacy compared |
| | with either monotherapy. A previous pilot trial of CRLX-101 demonstrated |
| | efficacy at 12 mg/kg with a two-weekly dosing schedule (NCT01612546). |
| | A further clinical study is currently ongoing in ovarian cancer to explore |
| | this combination |
| | The aim of CRLX-101-202 is to test the combination of CRLX-101 with |
| | PARP inhibition in two cancers where there is a high unmet need and |
| | both a rationale for, and current use of topoisomerase inhibitors, as well |
| | as evidence for sensitivity to PARP inhibitors: advanced/metastatic |
| | SCLC and ATM-ve gastric cancer |
| Primary | To investigate the efficacy, as defined by Best Objective Response Rate |
| Objectives | (ORR) of CRLX-101 in combination with Olaparib (an approved PARP |
| | inhibitor) in patients with: |
| | 1) ATM Negative relapsed advanced/metastatic Gastric tumours* |
| | 2) Small Cell Lung Cancer (SCLC) relapsed advanced/metastatic |
| | tumours* |
| | *(see Eligibility Criteria for definition of “relapse” for each tumour |
| | type/population) |
| Secondary | Median Progression-free survival (PFS) |
| Objectives | Overall Survival (OS) |
| | Duration of overall response (DOR) |
| | Disease control rate (DCR) |
| | Time to Progression (TTP) |
| | Safety profile of CRLX-101 in combination with olaparib |
| | determined by incidence & severity of treatment-emergent |
| | serious adverse events (CTCAE V5.0) |
| Exploratory | PK characteristics of CRLX-101 in combination with olaparib |
| Objectives | Investigate impact of HRD status on primary and selected |
| | secondary endpoints as analysed by post hoc stratification of |
| | patients into sub-groups of HRD status including gBRCA, |
| | SBRCA, BRCAness, e.g. as measured by ACT Genomics HRD |
| | panel, including at the level of individual HRD genes |
| | SCLC Arm - investigate the impact of varying lengths of initial |
| | chemotherapy free intervals (CFTs) following initial platinum |
| | chemotherapy on efficacy |
| | SCLC Arm - investigate the impact of initial response (CR/PR) |
| | to platinum chemotherapy on efficacy |
| Study Design | CRLX-101-202 is a multi-arm, multi-centre Phase 2 study to determine |
| | the efficacy, safety and tolerability of CRLX-101 in combination with |
| | Olaparib (an approved PARP inhibitor) in defined relapse populations of |
| | Gastric & Small Cell Lung Cancer tumours. |
| | Arm 1: ATM Negative relapsed advanced/metastatic Gastric tumours. |
| | This is a single-arm, two-stage study cohort, designed using Simon's |
| | two-stage methodology. |
| | Arm 2: Relapsed advanced/metastatic Small Cell Lung Cancer tumours. |
| | This is a single-arm, two-stage study cohort, designed using Simon's |
| | two-stage methodology. |
| | The treatment cohorts will open sequentially at the Sponsor's |
| | discretion and patients may be enrolled into each cohort |
| | concurrently. |
| | A schema of the trial design is depicted in FIG. 32. |
| Study | The term Investigational Agent in this study refers to CRLX-101. Study |
| Treatment | drug will be supplied by the Sponsor, Ellipses Pharma Ltd. |
| | CRLX-101 |
| | CRLX-101 will be supplied as a solution for iv administration, |
| | which will be presented in glass vials. |
| | CRLX-101 will be administered at a dose of 12 mg/m² on Days 1 |
| | and 15 of a 28 day cycle. |
| | Patients will receive pretreatment with steroids and/or |
| | antihistamine to mitigate hypersensitivity reactions to CRLX-101 |
| | seen in previous trials |
| | Instructions will be provided in the clinical study protocol on |
| | preparing patients for dosing to minimize bladder/urinary tract |
| | toxicity |
| | For initial Investigational Agent administration, a physician must |
| | be present at the site, or immediately available to respond to |
| | emergencies. |
| | In the event of toxicities, the dose of the Investigational Agent |
| | (CRLX-101) and/or Olaparib may be modified and a revised |
| | dose/schedule selected. Dose modification guidelines will be |
| | provided in the Study Protocol. |
| | Olaparib |
| | Olaparib bd oral 250 mg days 3-13 and 17-26 with a 48 hr window |
| | between CRLX-101 and olaparib dosing |
| | Duration of Study Treatment |
| | Patients may receive the combination of CRLX-101 with |
| | Olaparib for a planned 6 cycles of treatment. |
| | Patients with evidence of response (CR, PR, SD as per RECIST |
| | 1.1) after 6 cycles of treatment may continue with the |
| | combination or Olaparib monotherapy if continuing to experience |
| | clinical benefit as judged by the investigator until progression. |
| Population | 1) ATM Negative relapsed advanced/metastatic Gastric tumours* |
| Under Study | 2) Small Cell Lung Cancer (SCLC) relapsed advanced/metastatic |
| | tumours* |
| | *(see Eligibility Criteria for definition of ″relapse″ for each tumour |
| | type/population) |
| Inclusion | Arm 1: ATM Negative Relapsed advanced/metastatic Gastric Tumours |
| Criteria | Patients must meet all of Arm 1 specific Inclusion Criteria and all of the |
| | All Arms Eligibility Criteria to be eligible for inclusion in the study: |
| | ✓ Confirmed histological (cytological diagnosis excluded) of gastric |
| | adenocarcinoma as defined by the AJCC/UICC TNM staging |
| | classification (8th Ed, 2017) with archival tumour sample available. In |
| | the absence of an archival tumour biopsy sample, a tumour biopsy will |
| | need to be collected. |
| | ✓ HER2 & PDL-1 status known with no HER2 expression. |
| | ✓ ATM protein expression known by investigational use only |
| | immunohistochemical Ventana ATM (Y170) assay with ATM-negative |
| | status confirmed (as defined as less than 25% nuclear staining) |
| | ✓ Relapse, as defined as: clear, documented evidence of disease |
| | progression following only one line of previous therapy to consist of |
| | platinum containing doublets and triplets with at least 1 measurable |
| | lesion using CT/MRI as defined by RECIST 1.1 |
| | ✓ Age ≥18 years at the time of informed consent |
| | ✓ Eastern Cooperative Oncology Group (ECOG) performance status |
| | grade 0-1 |
| | Arm 2: Relapsed advanced/metastatic Small Cell Lung Cancer |
| | Patients must meet all of Arm 2 specific Inclusion Criteria and all of the |
| | All Arms Eligibility Criteria to be eligible for inclusion in the study: |
| | ✓ Confirmed histological (cytological diagnosis excluded) of small cell |
| | lung cancer (SCLC) as defined by the AJCC/UICC TNM staging |
| | classification (8th Ed, 2017) with archival tumour sample available. In |
| | the absence of an archival tumour biopsy sample, a tumour biopsy will |
| | need to be collected. |
| | ✓ PDL-1 status known. |
| | ✓ Relapse, as defined as: clear, documented evidence of disease |
| | progression following only one line of previous therapy to consist of a |
| | platinum containing doublets with or without PDL-1 immunotherapy as |
| | first line therapy with at least 1 measurable lesion using CT/MRI as |
| | defined by RECIST 1.1 |
| | ✓ Washout period: three weeks since last chemotherapy and/or four |
| | weeks since last immunotherapy |
| | ✓ Age ≥18 years at the time of informed consent |
| | ✓ Eastern Cooperative Oncology Group (ECOG) performance status |
| | grade 0-2 |
| | All Arms Inclusion Criteria |
| | ✓ Signed informed consent |
| | ✓ Registered within 4-6 weeks of diagnosis of radiological progression |
| | ✓ Estimated life expectancy >3 months |
| | Adequate haematological and organ function: |
| | ✓ Haemoglobin >9.0 g/dL |
| | ✓ Absolute neutrophil count (ANC) >1.5 × 109 |
| | ✓ Platelet count >100 × 109 |
| | ✓ Lymphocyte count ≥0.5 × 109 |
| | ✓ Total bilirubin <1.5 institutional upper limit normal (ULN) |
| | ✓ Serum albumin >2.5 g/dL |
| | ✓ AST and ALT < 2.5 × ULN, unless liver metastases are present in |
| | which case they must be <5 × ULN |
| | ✓ Patients not receiving anti-coagulant medication must have an INR of |
| | ~1.5 and an aPTT ~ 1.5 × ULN |
| | ✓ Serum creatinine ~1.5 × ULN or calculated creatinine clearance >50 |
| | mL/min (calculated using the Cockroft-Gault formula) for patients with |
| | creatinine levels above institutional normal |
| | ✓ In the opinion of the investigator, all other relevant medical conditions |
| | must be well-managed and stable for at least 28 days prior to first |
| | administration of study drug. |
| | ✓ Willing and able to participate in all required evaluations and |
| | procedures in this study protocol. |
| | ✓ For female subjects: each female subject of childbearing potential |
| | must agree to use a highly effective method of contraception (i.e., a |
| | method with less than 1% failure rate per year [e.g., sterilization, |
| | hormone implants, hormone injections, some intrauterine devices, |
| | vasectomized partner, or combined birth control pills]) from screening |
| | until 120 days after the last dose of CRLX-101 or Olaparib, whichever |
| | was taken last. Females of childbearing potential must have a negative |
| | serum pregnancy test at Screening and a negative serum or urine |
| | pregnancy test within 24 hours before each dose of CRLX-101 (and |
| | must not be lactating). Each female subject will be considered to be of |
| | childbearing potential unless she has been surgically sterilized by |
| | hysterectomy or bilateral tubal ligation/salpingectomy or has been |
| | postmenopausal for at least 1 year. |
| Exclusion | Arm 1: ATM Low Relapsed advanced/metastatic Gastric Tumours |
| Criteria | Patients must meet all of Arm 1 specific Exclusion Criteria and all of the |
| | All Arms Eligibility Criteria to be eligible for inclusion in the study: |
| | x Prior treatment with a topoisomerase I inhibitor |
| | Arm 2: Relapsed advanced/metastatic Small Cell Lung Cancer |
| | Patients must meet all of Arm 2 specific Exclusion Criteria and all of the |
| | All Arms Eligibility Criteria to be eligible for inclusion in the study: |
| | x Prior treatment with a topoisomerase I inhibitor |
| | All Arms Exclusion Criteria |
| | x Unresolved or unstable serious toxic side-effects of prior |
| | chemotherapy or radiotherapy, i.e. ≥ grade 2 per CTCAE (common |
| | terminology criteria for adverse events, v5.0) except fatigue, alopecia, |
| | infertility, or palliative radiotherapy within 6 weeks prior to |
| | randomization. |
| | x Clinical evidence of cerebral metastases or CNS involvement |
| | including leptomeningeal disease. Brain imaging for Gastric tumors |
| | must not be older than 12 weeks (at the start of screening). Brain |
| | imaging for Small Cell Lung Cancer tumors must not be older than 2 |
| | weeks (at the start of screening). Results of any unexpected or |
| | abnormal findings of brain imaging should be discussed with the |
| | Medical Monitor as part of the screening process. |
| | x History of previous malignancy that could interfere with response |
| | evaluation |
| | x Concurrent treatment with other systemic anti-cancer therapy or |
| | investigational anti-cancer drugs or within 3 weeks or five half-lives |
| | (whichever is longer) to the start of treatment with the Investigational |
| | Agent (CRLX-101) |
| | x Uncontrolled pleural effusion or pericardial effusion requiring recurrent |
| | drainage procedures (as defined as once monthly or more frequently) |
| | N.B - patients with indwelling catheters (e.g PleurX) are allowed. |
| | x Confirmed QTcF >470 ms on screening ECG or history of Torsades |
| | de pointes or history of congenital long QT syndrome |
| | x Any evidence of severe or uncontrolled systemic conditions (e.g., |
| | severe hepatic impairment) or current unstable or uncompensated |
| | respiratory or cardiac conditions which makes it undesirable for the |
| | patient to participate in the study or which could jeopardize compliance |
| | with the protocol |
| | x Any other concurrent severe and/or uncontrolled medical or surgical |
| | condition which, in the view of the investigator, could compromise the |
| | patient's participation in the study |
| | x Patients with active hepatitis infection (defined as having a positive |
| | hepatitis B surface antigen [HBsAg] test at screening) or hepatitis C. |
| | Patients with past hepatitis B virus (HBV) infection or resolved HBV |
| | infection (defined as having a negative HBsAg test and a positive |
| | antibody to hepatitis B core antigen [anti-HBc] antibody test) are eligible. |
| | Patients positive for hepatitis C virus (HCV) antibody are eligible only if |
| | polymerase chain reaction (PCR) is negative for HCV RNA |
| | x Active infection with SARS-Cov-2. All patients should be tested for |
| | active SARS-Cov-2 infection with an approved diagnostic kit. |
| | x Patients with active HIV infection or known history of HIV infection x |
| | Active infection requiring IV antibiotics within two weeks prior to |
| | treatment |
| | x Sexually active male patients must be willing to use barrier |
| | contraception (i.e., condoms with spermicide) with all sexual partners for |
| | the duration of the study and for 6 months after the last CRLX-101 |
| | administration. Where a sexual partner of a male participant is a |
| | ‘woman of child-bearing potential’, she must use a highly effective |
| | method contraceptive measures (see above definition) during her |
| | partner's participation in the study and for 6 months after her partner |
| | has received his last dose of CRLX-101. Men must not donate sperm |
| | for 6 months after the last dose of CRLX-101. |
| | x Women who are pregnant, breast-feeding or either unable to or refuse |
| | to use effective means of contraception during treatment |
| | x Any major surgical procedure (planned or anticipated) (in the |
| | investigator's judgement) within 2 weeks of the first dose of the |
| | Investigational Agent (CRLX-101) |
| | x Known contra-indications to topoisomerase I inhibitors or olaparib x |
| | Known hypersensitivity to topoisomerase I inhibitors (including |
| | camptothecin) or the excipients of the Investigational Agent |
| | (CRLX-101) |
| | x Known hypersensitivity to PARP inhibitors |
| | x Patients with a history, or features suggestive, of bone marrow |
| | dysplasia or myelodysplastic syndrome (MDS) or acute myeloid |
| | leukemia (AML). |
| | x The patient is unable to swallow capsules and/or has a surgical or |
| | anatomical condition that precludes swallowing and absorbing oral |
| | medication on an ongoing basis |
| | x Any other condition that would, in the investigator's judgement, |
| | contraindicate the patient's participation in the clinical study due to |
| | safety concerns or compliance with clinical study procedures |
| Efficacy | Gastric Arm: Objective Response Rate (ORR), as per RECIST 1.1 |
| Assessments | SCLC Arm: Objective Response Rate (ORR), as per RECIST 1.1 |
| | Follow up & Imaging: |
| | CT/MRI scan (chest/abdomen/pelvis) to assess disease status |
| | at baseline and then every 8 weeks (including RECIST |
| | measurements (version 1.1) |
| | Local CT reporting - for patients with measurable disease in |
| | follow-up, RECIST (v1.1) measurements must be taken. |
| | Further CT at suspicion of disease progression. |
| | Clinic follow-up at 2 and 4 weeks, then 4 weekly until disease |
| | progression |
| | Compliance measured using Olaparib patient diary |
| | Quality of life at 4 weekly study visits |
| | Following progression patients will be followed up as per |
| | standard practice or at the investigator's discretion and survival |
| | data collected |
| | End of treatment visit once patient discontinues (28 days post- |
| | last day of treatment). |
| | Follow up period: |
| | Treatment will continue until disease progression (by RECIST |
| | criteria V1.1) unacceptable toxicity, non-compliance with trial |
| | treatment and/or procedures, withdrawal of consent or death, |
| | whichever occurs first. |
| | Where no progressive disease occurs, follow up visits will |
| | continue for 12 months post-registration, then follow up for |
| | progression and survival until end of study. |
| Safety | Adverse events recording and toxicity grading using NCI CTCAE |
| Assessments | (V5.0) |
| | Laboratory - Full blood count, coagulation, electrolytes, renal |
| | and liver function |
| | Vital signs, physical examination ECG, performance status. |
| | Safety assessed by the nature of the AE, frequency, severity |
| | and relationship to the Investigational Agent (CRLX-101) |
| | Patients to be assessed for safety at screening, during the |
| | treatment period and for the follow-up period.' |
| | The investigator must notify the Sponsor immediately on any |
| | SAE |
| Statistical | Estimated sample size: |
| Considerations | Approximately 34 patients will be recruited across both arms in the first |
| | stage, with an additional 81 patients recruited in the second stage |
| | across both arms, making in total an estimated sample size of 115 |
| | patients. |
| | Sample size calculation: |
| | The sample size was calculated with 80% power and 5% one-sided |
| | type-1 error using the Simon's 2-stage optimal design which was used |
| | for both arms |
| | Arm 1 (Relapsed ATM Negative Gastric Cancer): if the ORR is 40%, to |
| | make sure that the lower end of the 95% one-sided Cl excludes 16%, 7 |
| | patients need to be recruited in the first stage. If there are at least 2 |
| | responders out of 7 evaluable patients, the study would continue to |
| | recruit 28 patients in total. Taking into account a potential drop-out rate |
| | of 20%, 9 patients will be recruited in stage 1. For stage 2, to make sure |
| | that there are 21 additional evaluable patients, 27 patients will be |
| | recruited to take |
| | into account a potential drop-out rate of 20%. The trial will be |
| | considered a success in this arm if the 95% one-sided CI for the |
| | observed proportion of responders excludes 16%. |
| | Arm 2 (Relapsed SCLC): if the ORR is 40%, to make sure that the lower |
| | end of the 95% one-sided Cl excludes 24%, 20 patients need to be |
| | recruited in the first stage. If there are at least 6 responders out of 20 |
| | evaluable patients, the study would continue to recruit 63 patients in |
| | total. Taking into account a potential drop-out rate of 20%, 25 patients |
| | will be recruited in stage 1. For stage 2, to make sure that there are 43 |
| | additional evaluable patients (total target evaluable sample size of 62), |
| | 54 patients will be recruited to take into account a potential drop-out rate |
| | of 20%. The trial will be considered a success in this arm if the 95% |
| | one-sided Cl for the observed proportion of responders excludes 24%. |
| | Statistical analysis of primary endpoints: |
| | The proportion of responders in each arm will be reported with |
| | its 95% one-sided Cl. |
| | All patients who receive at least one dose/cycle of CRLX-101 in |
| | combination with Olaparib will be included in the analysis |
| | population of the primary endpoint. |
| | Statistical analysis of secondary and exploratory endpoints: |
| | Proportions will be reported with 95% two-sided Cl. |
| | Time-to-event endpoints (PFS and OS) will be analysed using |
| | KM methods. Median survival time will be reported (if reached) |
| | with 95% two-sided Cl. Event rates at 6, 12 and 18 months will |
| | also be reported with 95% two-sided Cl. |

Schedule of Screening Assessments

| Procedure | Screening |
|---|---|
| Days (relative to first dose) | −28 to −1 |
| Informed consent | X |
| Demography | X |
| Inclusion/exclusion criteria | X |
| Medical history and cancer history | X |
| Prior anti-cancer therapy (specifically | X |
| chemotherapy free interval for SCLC) | |
| Prior/concomitant medications | X |
| Full physical examination | X |
| including height and weight | |
| ECOG performance status | X |
| Vital signs ₐ | X |
| 12-lead ECG | X |
| CT or MRI (RECIST) _{b} | X |
| Archival tumour sample (or | X |
| fresh biopsy if unavailable)^{c} | |
| Germline BRCA status _{d} | X |
| ATM/BRCA/HRD/PDL-1 | X |
| status in tumour tissue ₑ | |
| Haematology panel _{f} | X |
| Chemistry panel (including | X |
| renal & liver function tests) _{f} | |
| Urinalysis (dipstick) _{f} | X |
| Serum pregnancy test (women | X |
| of childbearing potential) _{g} | |
| Serum FSH test (confirm | X |
| menopausal status) _{f} | |
| Blood sample for | X |
| exploratory biomarkers | |

ₐ. Vital Signs: body temperature, pulse, respiratory rate and blood pressure
_{b}. Baseline CT or MRI should be performed of the chest, abdomen and pelvis with additional anatomy based on signs and symptoms of individual patients, utilizing RECIST v.1.1 criteria. Baseline assessments should be performed no more than 28 days before the start of study, and ideally should be performed as close as possible to the start of study drug. SCLC patients should have brain CT or MRI not more than 2 weeks prior to start of study to exclude brain disease, Gastric patients should have brain CT or MRI not more than 12 weeks prior to start of study to exclude brain disease.
^{c}. Archival tumour sample must be available. In the absence of an archival tumour biopsy, a tumour tissue biopsy will need to be collected prior to enrolment
_{d}. Records for all consenting patients will be reviewed to determine if BRCA1/BRCA2 mutation status is known. If patients do not have a known mutation status, then for consenting patients BRCA1/BRCA2 testing will be performed per standard institutional procedures. Status needs to be known prior to enrollment. Gastric cancer patients: records for all consenting patients will be reviewed to determine if tissue based BRCA/HRD status as per ACT Genomics panel is known. If patients do not have a known status, then for consenting patients HRD testing will be performed per standard institutional procedures. If testing not available locally testing can be performed centrally. Status needs to be known prior to enrollment. In addition ATM protein expression status as per ATM protein expression investigational use only immunohistochemical Ventana ATM (Y170) must be known prior to enrollment. Small cell lung cancer patients: records for all consenting patients will be reviewed to determine if tissue based BRCA/HRD status as per ACT Genomics panel is known. If patients do not have a known status, then for consenting patients HRD testing will be performed per standard institutional procedures. If testing not available locally testing can be performed centrally. Status needs to be known prior to enrollment. In addition records for all consenting patients will be reviewed to determine if tissue based PDL-1 status. If patients do not have a known status, then for consenting patients PDL-1 testing will be performed per standard institutional procedures. If testing not available locally testing can be performed centrally. Status needs to be known prior to enrollment.
ₑ. To be performed locally
_{f}. A serum pregnancy test should be performed within 72 hours prior to C1D1 and confirmed as negative for dosing to commence. Test to be performed locally.
Screen Failures: Any patient who signed the ICF, but failed to start treatment for any reason, will be considered a screen failure. The demographic information and reason for screen fail will be captured in the database for screen fail patients. No other data will be entered unless the patient experienced any AEs during Screening, which would be reported in the usual manner via eCRF AE page.

Schedule of Treatment Period Assessments

| | | | | | | | | | | Cycle 3 and | | |
|---|---|---|---|---|---|---|---|---|---|---|---|---|
| | Cycle 1 | | | | | | Cycle 2 | | | subsequent cycles^{e} | | |
| Day | D1 | D3 | D8 | D15 | D21 | D28 | D1 | D15 | D28 | D1 | D15 | D28 |

| | |
|---|---|
| Adverse events | Review from consent until 30 days after last dose, grade and document according to NCI CTCAE v.5^{k} |
| Concomitant medications | Review from screening until Follow up |

Safety Assessments

| | | | | | | | | | | | | |
|---|---|---|---|---|---|---|---|---|---|---|---|---|
| Complete physical | X | | | | | | X | | | X | | |
| examination ^{b} | | | | | | | | | | | | |
| Brief physical | | X | X | X | X | X | | X | X | | X | X |
| examination | | | | | | | | | | | | |
| Weight | X | | | | | | X | | | X | | |
| Vital signs ^{a} | X | X | X | X | X | X | X | X | X | X | X | X |
| 12-lead ECG | X | | | | | | X | | | X | | |
| ECOG performance | X | | | | | | X | | | X | | |
| status | | | | | | | | | | | | |
| Haematology panel ⁱ | X | | X | X | X | X | X | X | | X | X | |
| Chemistry panel | X | | | X | X | X | X | X | | X | X | |
| Urinalysis (dipstick) | X | | | X | | | X | X | | X | X | |
| Urine pregnancy test | X | | | | | | X | | | X | | |

Tumour Assessment

| | | | | | |
|---|---|---|---|---|---|
| CT or MRI (RECIST) ^{c} | | | | X _{d} | X ^{d} |
| PK | | | | | |
| CRLX-101 Plasma PK ^{g,f} | X | X | X | | |

Quality of Life & Compliance

| | | | | | |
|---|---|---|---|---|---|
| QoL Forms ₕ | X | | X | | X |
| Investigator to check | | X | | X | |
| patient diary for Olaparib | | | | | |
| compliance | | | | | |

^{a} Vital Signs: body temperature, respiratory rate, pulse and blood pressure
^{b} Height only needs to be taken once, during screening
^{c} Tumour assessment; CT or MRI scans of abdomen and pelvis are to be repeated every 8 weeks (every two cycles) (+/−7 days) up to 12 months and then every 12 weeks (+/−2 weeks) utilising RECIST v.1.1 criteria; the same type of equipment that was used for baseline to rule out CNS involvement.
^{d} When requested outside of 8 weekly routine evaluation on clinical suspicion, results of tumour assessments must be available before day 1 of next scheduled cycle in order to exclude disease progression. Copies of all scans will be collected and held centrally
^{e}Patients can continue receiving CRLX-101 in combination with Olaparib until confirmed disease progression, provided they have not met any other discontinuation criteria and the Investigator believes it is in the patient's best interest
^{f} PK timepoints: C1D1 pre-infusion, end of infusion, 2 hours, 4 hours, 8 hours, 16 hours, 24 hours, 48 hours and C1D15 pre-infusion and end of infusion
^{g} Two separate samples are needed for plasma PK, one to measure free Camptothecin and the other to measure total Camptothecin
ₕ QoL Forms (EORTC Quality-of-Life Questionnaire QLQ-C30, QLQ-OV-28 and EQ-5D)
ⁱ FBC & clotting function. Haematology samples are to be taken more frequently during C1 due to risk of neutropenia and other haematology toxicity for both CRLX-101 and for patients in Phase 2B SOC chemotherapy. If a patient has significant neutropenia or other significant haematological abnormalities during any cycle, more frequent monitoring should occur as per local guidelines.
^{j} To include renal & liver function tests.
^{k}Any patient presenting with a grade 3 or greater treatment-related AE should have all relevant tests re-assessed at least 48-72 until recovery to less than grade 2
Any assessments required on C1D1 that are also performed as part of the screening evaluations, do not need to be repeated on C1D1 if the screening/baseline evaluation is to be used throughout. SCLC patients should have brain CT or MRI must be undertaken at the screening data was obtained within 72 hours of the first dose of study drug. During the study visits, all tests and/or procedures should occur on schedule whenever possible. A three day window will be allowed for laboratory procedures and ECG, a one week window (7 days) for radiological tumour assessments or two weeks after one year, a one day window for clinical assessments (ECOG PS, physical examination, vital signs).

Schedule of Follow-up Assessments

| | Visit | | | |
|---|---|---|---|---|
| | End of Treatment (EOT) | | Long Term Follow-up | |
| | | 30-Day Post Last | Tumour Response | |
| | EOT Visit ^{a} | Dose ^{b} | FU ^{c} | Survival FU ^{d} |
| | 7 Days | | Every 12 weeks | |
| Procedure | | +/− 3 days | +1/−2 weeks | |
| Survival contact | | X | X | X |
| Adverse events | X | X | | |
| Concomitant medications | X | X | | |
| Subsequent anti-cancer treatment | X | X | X | X |
| Brief physical examination including weight | X | X | X | |
| Vital signs | X | X | | |
| Tumour Assessment | X ^{e} | | X | |
| 12-lead ECG | X | If abnormal at EOT | | |
| ECOG performance status | X | X | | |
| Hematology panel | X | X | | |
| Chemistry panel | X | X | | |
| Urinalysis | X | X | | |
| Pregnancy test (serum, plasma or urine, | X | X | | |
| women of childbearing potential) | | | | |
| QoL Forms ^{f} | X | X | X | |

^{a}. At the time a patient discontinues study drug, an End of Treatment (EOT) visit should be scheduled as soon as possible, preferably within 7 days of the last dose of study drug or within 7 days of the decision to permanently discontinue study drug
^{b}. All patients will have a 30-Day Post Last Dose follow-up visit (+/− 3 days)
^{c}. For patients who have not progressed, follow-up should be performed every 12 weeks (+/− 2 weeks) until radiological disease progression
^{d}. For patients who have progressed, follow-up, via telephone, email or letter, should be performed every 12 weeks (+/− 4 weeks) until death
^{e}. For patients who have stopped study treatment for reasons other than Progressive Disease or withdrawal of consent, a CT/MRI scan will be performed at the EOT visit, providing a CT/MRI scan has not been performed within the previous 6 weeks
^{f}. QOL Forms (EORTC Quality-of-Life Questionnaire QLQ-C30, QLQ-OV-28 and EQ-5D)

The disclosure also comprises the following clauses, which may be claimed:
1. A cyclodextrin-containing polymer-topoisomerase inhibitor conjugate for use in treating ovarian cancer in a subject;
   wherein the use is in combination with a poly (ADP-ribose) polymerase (PARP) inhibitor; and
   wherein the subject has previously undergone chemotherapy comprising a platinum-based chemotherapeutic agent prior to said use.
2. The cyclodextrin-containing polymer-topoisomerase inhibitor conjugate for use according to clause 1, wherein the subject has not previously undergone therapy comprising a PARP inhibitor.
3. The cyclodextrin-containing polymer-topoisomerase inhibitor conjugate for use according to clause 1 or 2, wherein the use is the second line of chemotherapy for treating ovarian cancer in a subject.
4. The cyclodextrin-containing polymer-topoisomerase inhibitor conjugate for use according to any preceding clause, wherein the subject is refractory or resistant to the platinum-based chemotherapeutic agent, preferably wherein the subject is resistant to the platinum-based chemotherapeutic agent.
5. The cyclodextrin-containing polymer-topoisomerase inhibitor conjugate for use according to clause 1, wherein the use is the third or later line of chemotherapy for treating ovarian cancer in a subject.
6. The cyclodextrin-containing polymer-topoisomerase inhibitor conjugate for use according to clause 1 or 5, wherein the subject has previously undergone therapy comprising a PARP inhibitor.
7. The cyclodextrin-containing polymer-topoisomerase inhibitor conjugate for use according to clause 6, wherein the therapy comprising the PARP inhibitor is a maintenance therapy.
8. The cyclodextrin-containing polymer-topoisomerase inhibitor conjugate for use according to clause 7, wherein therapy comprising the PARP inhibitor comprises a PARP inhibitor selected from olaparib, veliparib, niraparib, rucaparib, talazoparib (preferably olaparib, niraparib or rucaparib), or a pharmaceutically acceptable salt of the foregoing.
9. The cyclodextrin-containing polymer-topoisomerase inhibitor conjugate for use according to clause 8, wherein therapy comprising the PARP inhibitor comprises olaparib, or a pharmaceutically acceptable salt thereof.
10. The cyclodextrin-containing polymer-topoisomerase inhibitor conjugate for use according to any one of clauses 7 to 9, wherein the maintenance therapy is the most recent therapy prior to said use.
11. The cyclodextrin-containing polymer-topoisomerase inhibitor conjugate for use according to clause 10, wherein the subject has previously undergone chemotherapy comprising a platinum-based chemotherapeutic agent and the PARP-based maintenance therapy as a next therapy after the chemotherapy comprising a platinum-based chemotherapeutic agent, said PARP-based maintenance therapy being their most recent therapy prior to said use.
12. The cyclodextrin-containing polymer-topoisomerase inhibitor conjugate for use according to any one of clauses 7 to 11, wherein the cancer has progressed within about 9 months (optionally within 6 to about 9 months) after beginning the PARP-based maintenance therapy.
13. The cyclodextrin-containing polymer-topoisomerase inhibitor conjugate for use according to any one of clauses 7 to 12, wherein the patient has undergone PARP-based maintenance therapy for at least about 6 months, optionally at least about 9 months, optionally at least about 12 months prior to said use.
14. The cyclodextrin-containing polymer-topoisomerase inhibitor conjugate for use according to any preceding clause, wherein the subject is stable, refractory or resistant to the platinum-based chemotherapeutic agent, preferably wherein the subject is stable or resistant to the platinum-based chemotherapeutic agent.
15. The cyclodextrin-containing polymer-topoisomerase inhibitor conjugate for use according to any preceding clause, wherein the subject has previously undergone chemotherapy comprising a platinum-based chemotherapeutic agent and at least one other chemotherapy comprising a different chemotherapeutic agent (optionally a different platinum-based chemotherapeutic agent).
16. The cyclodextrin-containing polymer-topoisomerase inhibitor conjugate for use according to any preceding clause, wherein the subject has previously undergone chemotherapy comprising a platinum-based chemotherapeutic agent selected from carboplatin, oxaliplatin and cisplatin, optionally carboplatin.
17. The cyclodextrin-containing polymer-topoisomerase inhibitor conjugate for use according to any preceding clause, wherein the subject has previously undergone:
   a line of chemotherapy comprising a platinum-based chemotherapeutic agent selected from carboplatin, oxaliplatin and cisplatin (optionally carboplatin); and
   at least one other line of chemotherapy comprising a platinum-based chemotherapeutic agent selected from carboplatin, oxaliplatin and cisplatin (optionally oxaliplatin);
   wherein the at least one other line is with the same or different platinum-based chemotherapeutic agent.
18. The cyclodextrin-containing polymer-topoisomerase inhibitor conjugate for use according to any preceding clause, wherein the topoisomerase inhibitor is camptothecin or a camptothecin derivative.
19. The cyclodextrin-containing polymer-topoisomerase inhibitor conjugate for use according to any preceding clause, wherein the cyclodextrin-containing polymer-topoisomerase inhibitor conjugate comprises the following repeat unit:

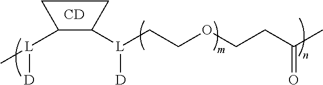

wherein CD is a cyclodextrin moiety;
D is a camptothecin moiety, such as a camptothecin moiety bound to a linker (e.g. glycine), for example optionally substituted:

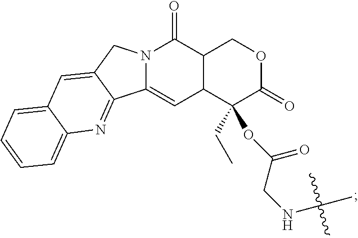 [SMILES: *NCC(=O)O[C@]1(CC)C(=O)OCC2C(=O)N3Cc4cc5ccccc5nc4C3=CC21]

L is a linker comprising an amino acid moiety, such as alanine, arginine, histidine, lysine, aspartic acid, glutamic acid, serine, threonine, asparagine, glutamine, cysteine, glycine, proline, isoleucine, leucine, methionine, phenylalanine, tryptophan, tyrosine and valine (e.g. comprising a cysteine moiety);
n is between about 10 and 20, such as about 12 to 16, preferably about 14; and
m is between about 40 and 100, optionally between about 60 and 90, optionally between about 70 and 80, preferably about 75 and 80, such as about 77.
20. The cyclodextrin-containing polymer-topoisomerase inhibitor conjugate for use according to any preceding clause, wherein the cyclodextrin-containing polymer-topoisomerase inhibitor conjugate comprises the following repeat unit:

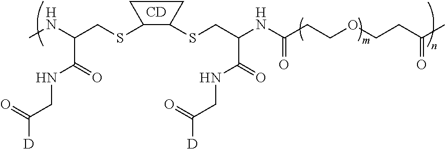

wherein CD is a cyclodextrin moiety;
D is a camptothecin moiety, preferably optionally substituted

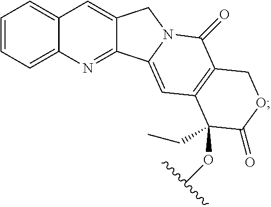 [SMILES: *O[C@]1(CC)C(=O)OCc2c1cc1n(c2=O)Cc2cc3ccccc3nc2-1]

n is between about 10 and 20, such as about 12 to 16, preferably about 14; and
m is between about 40 and 100, optionally between about 60 and 90, optionally between about 70 and 80, preferably about 75 and 80, such as about 77.
21. The cyclodextrin-containing polymer-topoisomerase inhibitor conjugate for use according to any preceding clause, wherein the cyclodextrin-containing polymer-topoisomerase inhibitor conjugate is CRLX-101.
22. The cyclodextrin-containing polymer-topoisomerase inhibitor conjugate for use according to any preceding clause, wherein the cyclodextrin-containing polymer-topoisomerase inhibitor conjugate is nanoparticulate.
23. The cyclodextrin-containing polymer-topoisomerase inhibitor conjugate for use according to any preceding clause, wherein the use is in combination with a PARP inhibitor selected from olaparib, veliparib, niraparib, rucaparib and talazoparib (preferably olaparib, niraparib or rucaparib), or a pharmaceutically acceptable salt of any of the foregoing.
24. The cyclodextrin-containing polymer-topoisomerase inhibitor conjugate for use according to clause 23, wherein the PARP inhibitor is olaparib, or a pharmaceutically acceptable salt thereof.
25. The cyclodextrin-containing polymer-topoisomerase inhibitor conjugate for use according to any preceding clause, wherein the use comprises administration of the cyclodextrin-containing polymer-topoisomerase inhibitor conjugate and the PARP inhibitor sequentially.
26. A cyclodextrin-containing polymer-topoisomerase inhibitor conjugate for use in treating cancer in a subject;
   wherein the use is in combination with a poly (ADP-ribose) polymerase inhibitor; and
   wherein the cancer is selected from gastric, colorectal, cervical and pancreatic (preferably gastric cancer).
27. The cyclodextrin-containing polymer-topoisomerase inhibitor conjugate for use according to clause 26, comprising the features of any one of clauses 1 to 25.
28. The cyclodextrin-containing polymer-topoisomerase inhibitor conjugate for use according to any preceding clause, wherein the use is in combination with a further agent selected from any of the chemotherapeutic agents; hormone and/or steroids; anti-microbials; agents or procedure to mitigate potential side effects from the agent compositions such as cystitis, diarrhea, nausea and vomiting; anti-hypersensitivity agents; an agent that increases urinary excretion and/or neutralizes one or more urinary metabolite; antidiarrheal agents; antiemetic agents; immunosuppressive agents; antihistamines; anti-inflammatories; and antipyretics such as any of those outlined under the heading “ADDITIONAL THERAPEUTIC AGENTS”.
29. The cyclodextrin-containing polymer-topoisomerase inhibitor conjugate for use according to any preceding clause, wherein the cancer is advanced or metastatic ovarian cancer (preferably stage III or stage IV).
30. A method for treating ovarian cancer in a subject, the method comprising:
   administering to a subject in need thereof a cyclodextrin-containing polymer-topoisomerase inhibitor conjugate;
   in combination with a poly (ADP-ribose) polymerase (PARP) inhibitor;
   wherein the subject has previously undergone chemotherapy comprising a platinum-based chemotherapeutic agent.
31. The method according to clause 30, comprising the features of any one of clauses 1 to 25.
32. A method for treating cancer in a subject, the method comprising:
   administering to a subject in need thereof a cyclodextrin-containing polymer-topoisomerase inhibitor conjugate;
   in combination with a poly (ADP-ribose) polymerase (PARP) inhibitor;
wherein the cancer is selected from gastric, colorectal, cervical, pancreatic.
33. The method according to clause 32, comprising the features of any one of clauses 1 to 25.
34. The cyclodextrin-containing polymer-topoisomerase inhibitor conjugate for use according to any one of claims 26 to 29 or the method for treating cancer according to any one of claims 30 to 33, wherein the cancer is gastric cancer.

This disclosure is not limited to particular embodiments described, as such may, of course, vary. It is also to be understood that the terminology used herein is for the purpose of describing particular embodiments only, and is not intended to be limiting, since the scope of the present application may be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present application.

1. A cyclodextrin-containing polymer-topoisomerase inhibitor conjugate for use in treating ovarian cancer in a subject;
wherein the use is in combination with a poly (ADP-ribose) polymerase (PARP) inhibitor; and
wherein the subject has previously undergone chemotherapy comprising a platinum-based chemotherapeutic agent prior to said use.

2. The cyclodextrin-containing polymer-topoisomerase inhibitor conjugate for use according to claim 1, wherein the use is the second line of chemotherapy for treating ovarian cancer in a subject.

3. The cyclodextrin-containing polymer-topoisomerase inhibitor conjugate for use according to claim 1, wherein the use is the third or later line of chemotherapy for treating ovarian cancer in a subject.

4. The cyclodextrin-containing polymer-topoisomerase inhibitor conjugate for use according to any preceding claim, wherein the cancer is advanced or metastatic ovarian cancer (preferably stage III or stage IV).

5. The cyclodextrin-containing polymer-topoisomerase inhibitor conjugate for use according to any preceding claim, wherein the subject is refractory or resistant to the platinum-based chemotherapeutic agent, preferably wherein the subject is resistant to the platinum-based chemotherapeutic agent.

6. A cyclodextrin-containing polymer-topoisomerase inhibitor conjugate for use in treating cancer in a subject;
wherein the use is in combination with a poly (ADP-ribose) polymerase inhibitor; and
wherein the cancer is selected from gastric, colorectal, cervical and pancreatic.

7. The cyclodextrin-containing polymer-topoisomerase inhibitor conjugate for use according to claim 6, wherein the cancer is gastric cancer.

8. The cyclodextrin-containing polymer-topoisomerase inhibitor conjugate for use according to any preceding claim, wherein the subject has not previously undergone therapy comprising a PARP inhibitor.

9. The cyclodextrin-containing polymer-topoisomerase inhibitor conjugate for use according to any one of claims 1 to 7, wherein the subject has previously undergone therapy comprising a PARP inhibitor.

10. The cyclodextrin-containing polymer-topoisomerase inhibitor conjugate for use according to any preceding claim, wherein the use is in combination with a PARP inhibitor selected from olaparib, veliparib, niraparib, rucaparib and talazoparib (preferably olaparib, niraparib or rucaparib), or a pharmaceutically acceptable salt of any of the foregoing.

11. The cyclodextrin-containing polymer-topoisomerase inhibitor conjugate for use according to claim 10, wherein the PARP inhibitor is olaparib, or a pharmaceutically acceptable salt thereof.

12. The cyclodextrin-containing polymer-topoisomerase inhibitor conjugate for use according to any preceding claim, wherein the cyclodextrin-containing polymer-topoisomerase inhibitor conjugate comprises the following repeat unit: 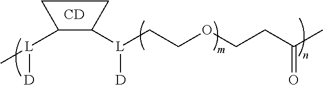
wherein CD is a cyclodextrin moiety;
D is a camptothecin moiety, such as a camptothecin moiety bound to a linker (e.g. glycine), for example wherein D is optionally substituted: 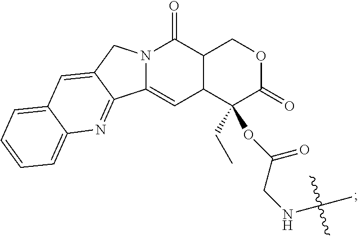 [SMILES: *NCC(=O)O[C@]1(CC)C(=O)OCC2C(=O)N3Cc4cc5ccccc5nc4C3=CC21]
L is a linker comprising an amino acid moiety, such as alanine, arginine, histidine, lysine, aspartic acid, glutamic acid, serine, threonine, asparagine, glutamine, cysteine, glycine, proline, isoleucine, leucine, methionine, phenylalanine, tryptophan, tyrosine and valine (e.g. comprising a cysteine moiety);
n is between about 10 and 20, such as about 12 to 16, preferably about 14; and
m is between about 40 and 100, optionally between about 60 and 90, optionally between about 70 and 80, preferably about 75 and 80, such as about 77.

13. The cyclodextrin-containing polymer-topoisomerase inhibitor conjugate for use according to any preceding claim, wherein the cyclodextrin-containing polymer-topoisomerase inhibitor conjugate comprises the following repeat unit: 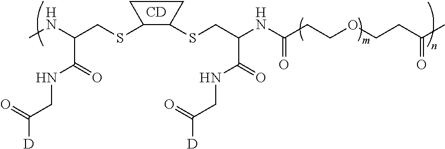
wherein CD is a cyclodextrin moiety;
D is a camptothecin moiety, preferably wherein D is optionally substituted 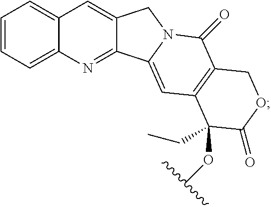 [SMILES: *O[C@]1(CC)C(=O)OCc2c1cc1n(c2=O)Cc2cc3ccccc3nc2-1]
n is between about 10 and 20, such as about 12 to 16, preferably about 14; and
m is between about 40 and 100, optionally between about 60 and 90, optionally between about 70 and 80, preferably about 75 and 80, such as about 77.

14. The cyclodextrin-containing polymer-topoisomerase inhibitor conjugate for use according to any preceding claim, wherein the cyclodextrin-containing polymer-topoisomerase inhibitor conjugate is CRLX-101.

15. The cyclodextrin-containing polymer-topoisomerase inhibitor conjugate for use according to any preceding claim, wherein the cyclodextrin-containing polymer-topoisomerase inhibitor conjugate is CRLX-101 and wherein the poly (ADP-ribose) polymerase inhibitor is olaparib, or a pharmaceutically acceptable salt thereof.

16. The cyclodextrin-containing polymer-topoisomerase inhibitor conjugate for use according to any preceding claim, wherein the cyclodextrin-containing polymer-topoisomerase inhibitor conjugate is nanoparticulate.
